(19) United States
(12) Patent Application Publication
(10) Pub. No.: US 2020/0199523 A1
(43) Pub. Date: Jun. 25, 2020

(54) MEDIA FOR MICROORGANISM CULTURE AND RELATED COMPOSITIONS AND METHODS

(71) Applicant: Massachusetts Institute of Technology, Cambridge, MA (US)

(72) Inventors: J. Christopher Love, Somerville, MA (US); Catherine Bartlett Matthews, Cambridge, MA (US); Kerry R. Love, Somerville, MA (US); Angel Kuo, Cambridge, MA (US)

(73) Assignee: Massachusetts Institute of Technology, Cambridge, MA (US)

(21) Appl. No.: 16/500,071

(22) PCT Filed: Mar. 30, 2018
(86) PCT No.: PCT/US2018/025406

§ 371 Date: Oct. 1, 2019

Related U.S. Application Data
(60) Provisional application No. 62/480,416, filed on Apr. 1, 2017.
(60) Provisional application No. 62/644,820, filed on Mar. 19, 2018.

(51) Int. Cl. C12N 1/16; C12P 21/02

(52) U.S. Cl. CPC C12N 1/16; C12P 21/02; C12N 2500/34; C12N 2500/14; C12N 2500/33; C12N 2500/16

(57) ABSTRACT

Disclosed is media for cultivating cells, e.g., Pichia Pastoris cells, as well as cultures containing cells, methods for making and using the media, and kits comprising the media. The media is particularly useful in the context of therapeutic protein production.

### RELATED APPLICATION

This application is a national stage filing under 35 U.S.C. § 371 of international PCT application, PCT/US2018/025406, filed Mar. 30, 2018, which claims the benefit of the filing dates of U.S. Provisional Application No. 62/480,416, filed on Apr. 1, 2017, and U.S. Provisional Application No. 62/644,820, filed on Mar. 19, 2018, the content of each of which is incorporated by reference in its entirety.

### GOVERNMENT SUPPORT

This invention was made with Government support under Contract No. N66001-13-C-4025 awarded by the Space and Naval Warfare Systems Center. The Government has certain rights in the invention.

### BACKGROUND OF INVENTION

Pichia pastoris is a methylotrophic yeast that is commonly used as an expression system for the production of heterologous proteins. Pichia is an ideal organism for the production of heterologous proteins at least because (i) it is capable of many of the same post-translational modifications of higher eukaryotic host cells; (ii) its high growth rates and high cell densities provide for high levels of protein production; (iii) it has a highly stable genome for extended production; (iv) it has highly regulatable promoters, such as AOX1 and AOX2, that provide tight regulation of recombinant gene expression; and (v) it has been engineered to produce mammalian glycosylation patterns.

### SUMMARY OF INVENTION

Provided herein are unexpectedly improved media for culturing microorganisms, e.g., Pichia pastoris, as well as cell cultures, methods of making and using the media, and kits. These media are particularly suited for the cultivation of heterologous protein-producing microorganisms, e.g., microorganisms engineered to express heterologous proteins. The media allow for increased growth of the microorganisms as well as increased protein production. Features of the media that provide for these improved properties relative to existing chemically defined media include one or more of: i) reduced ammonium levels; ii) one or more lipids; iii) glutamine; iv) arginine; v) asparagine and/or vi) one or more vitamins.

Accordingly, in one aspect, provided herein are cell culture media. The cell culture media comprises

(a) KH₂PO₄;

(b) MgSO₄;

(c) CaCl₂; and

(d) one or more of:
(i) ammonium at a concentration of 0.2-3.7 g/L;
(ii) one or more of biotin, calcium pantothenate, nicotinic acid, inositol, thiamin HCl, pyridoxine HCl, and para-aminobenzoic acid;
(iii) glutamine;
(iv) arginine; and
(v) one or more of arachidonic fatty acid, linoleic fatty acid, linolenic fatty acid, myristic fatty acid, oleic fatty acid, palmitic fatty acid, stearic fatty acid, and cholesterol, wherein the g/L denote concentration when the media is diluted in a solvent.

In some embodiments, the media comprises ammonium at a concentration of 0.2-3.7 g/L and one or more of arachidonic fatty acid, linoleic fatty acid, linolenic fatty acid, myristic fatty acid, oleic fatty acid, palmitic fatty acid, stearic fatty acid, and cholesterol. In certain embodiments, the media comprises ammonium at a concentration of 0.2-3.7 g/L and glutamine. In some embodiments, the media comprises ammonium at a concentration of 0.2-3.7 g/L, glutamine, and one or more of arachidonic fatty acid, linoleic fatty acid, linolenic fatty acid, myristic fatty acid, oleic fatty acid, palmitic fatty acid, stearic fatty acid, and cholesterol. In some embodiments, the media comprises 2, 3, 4, or all of: (i) ammonium at a concentration of 0.2-3.7 g/L; (ii) one or more of biotin, calcium pantothenate, nicotinic acid, inositol, thiamin HCl, pyridoxine HCl, and para-aminobenzoic acid; (iii) glutamine; (iv) arginine; and (v) one or more of arachidonic fatty acid, linoleic fatty acid, linolenic fatty acid, myristic fatty acid, oleic fatty acid, palmitic fatty acid, stearic fatty acid, and cholesterol.

In another aspect, provided herein are cell culture media. The cell culture media comprises

(a) KH₂PO₄;

(b) MgSO₄;

(c) CaCl₂; and

(d) one or more of:
(i) ammonium at a concentration of 0.2-3.7 g/L;
(ii) one or more of biotin, calcium pantothenate, nicotinic acid, inositol, thiamin HCl, pyridoxine HCl, and para-aminobenzoic acid;
(iii) one or more amino acids; and
(iv) one or more of arachidonic fatty acid, linoleic fatty acid, linolenic fatty acid, myristic fatty acid, oleic fatty acid, palmitic fatty acid, stearic fatty acid, and cholesterol, wherein the g/L denote concentration when the medium is diluted in a solvent.

In some embodiments, the media comprises 2, 3, 4, 5, 6, 7, or all of arachidonic fatty acid, linoleic fatty acid, linolenic fatty acid, myristic fatty acid, oleic fatty acid, palmitic fatty acid, stearic fatty acid, and cholesterol. In certain embodiments, the media comprises one or more of: arachidonic fatty acid at a concentration of 5 μg/L-1 g/L, optionally at a concentration of about 20 μg/L or about 250 mg/L; linoleic fatty acid at a concentration of 10 μg/L-1 g/L, optionally at a concentration of about 100 μg/L or about 250 mg/L; linolenic fatty acid at a concentration of 10 μg/L-1 g/L, optionally at a concentration of about 100 μg/L or about 250 mg/L; myristic fatty acid at a concentration of 10 μg/L-1 g/L, optionally at a concentration of about 100 μg/L or about 250 mg/L; oleic fatty acid at a concentration of 10 μg/L-1 g/L, optionally at a concentration of about 100 μg/L or about 250 mg/L; palmitic fatty acid at a concentration of 10 μg/L-1 g/L, optionally at a concentration of about 100 μg/L or about 250 mg/L; stearic fatty acid at a concentration of 10 μg/L-1 g/L, optionally at a concentration of about 100 μg/L or about 250 mg/L; and cholesterol at a concentration of 1-5 g/L, optionally at a concentration of about 2.2 g/L; wherein the g/L denote concentration when the media is diluted in a solvent. In some embodiments, the media comprises 2, 3, 4, 5, 6, 7, or all of: arachidonic fatty acid at a concentration of 5 μg/L-1 g/L, optionally at a concentration of about 20 μg/L or about 250 mg/L; linoleic fatty acid at a concentration of 10 μg/L-1 g/L, optionally at a concentration of about 100 μg/L or about 250 mg/L; linolenic fatty acid at a concentration of 10 μg/L-1 g/L, optionally at a concentration of about 100 μg/L or about 250 mg/L; myristic fatty acid at a concentration of 10 μg/L-1 g/L, optionally at a concentration of about 100 μg/L or about 250 mg/L; oleic fatty acid at a concentration of 10 μg/L-1 g/L, optionally at a concentration of about 100 μg/L or about 250 mg/L; palmitic fatty acid at a concentration of 10 μg/L-1 g/L, optionally at a concentration of about 100 μg/L or about 250 mg/L; stearic fatty acid at a concentration of 10 μg/L-1 g/L, optionally at a concentration of about 100 μg/L or about 250 mg/L; and cholesterol at a concentration of 1-5 g/L, optionally at a concentration of about 2.2 g/L; wherein the g/L denote concentration when the media is diluted in a solvent.

In some embodiments, the medium comprises one or more amino acids. In some embodiments, the one or more amino acids comprises one or more of glutamine, arginine, and asparagine. In some embodiments, the one or more amino acids comprises 2 or 3 of glutamine, arginine, and asparagine. In some embodiments, the one or more amino acids comprises one or more of: glutamine at a concentration of 12.5 mM-50 mM, optionally at a concentration of about 25 mM; arginine at a concentration of 12.5 mM-50 mM, optionally at a concentration of about 25 mM; and asparagine at a concentration of 12.5 mM-50 mM, optionally at a concentration of about 25 mM. In some embodiments, the one or more amino acids comprises 2 or 3 of: glutamine at a concentration of 12.5 mM-50 mM, optionally at a concentration of about 25 mM; arginine at a concentration of 12.5 mM-50 mM, optionally at a concentration of about 25 mM; and asparagine at a concentration of 12.5 mM-50 mM, optionally at a concentration of about 25 mM.

In some embodiments, the medium is for producing human growth hormone (hGH). In said embodiments, the one or more amino acids comprises one or more of histidine, alanine, glutamine, aspartate, serine, glutamate, proline, asparagine, glycine, and arginine. In some embodiments, the one or more amino acids comprises 2, 3, 4, 5, 6, 7, 8, 9, or all of histidine, alanine, glutamine, aspartate, serine, glutamate, proline, asparagine, glycine, and arginine. In some embodiments, the one or more amino acids comprises one or more of: histidine at a concentration of 12.5 mM-50 mM, optionally at a concentration of about 25 mM; alanine at a concentration of 12.5 mM-50 mM, optionally at a concentration of about 25 mM; glutamine at a concentration of 12.5 mM-50 mM, optionally at a concentration of about 25 mM; aspartate at a concentration of 12.5 mM-50 mM, optionally at a concentration of about 25 mM; serine at a concentration of 12.5 mM-50 mM, optionally at a concentration of about 25 mM; glutamate at a concentration of 12.5 mM-50 mM, optionally at a concentration of about 25 mM; proline at a concentration of 12.5 mM-50 mM, optionally at a concentration of about 25 mM; asparagine at a concentration of 12.5 mM-50 mM, optionally at a concentration of about 25 mM; glycine at a concentration of 12.5 mM-50 mM, optionally at a concentration of about 25 mM; and arginine at a concentration of 12.5 mM-50 mM, optionally at a concentration of about 25 mM. In some embodiments, the one or more amino acids comprises 2, 3, 4, 5, 6, 7, 8, 9, or all of: histidine at a concentration of 12.5 mM-50 mM, optionally at a concentration of about 25 mM; alanine at a concentration of 12.5 mM-50 mM, optionally at a concentration of about 25 mM; glutamine at a concentration of 12.5 mM-50 mM, optionally at a concentration of about 25 mM; aspartate at a concentration of 12.5 mM-50 mM, optionally at a concentration of about 25 mM; serine at a concentration of 12.5 mM-50 mM, optionally at a concentration of about 25 mM; glutamate at a concentration of 12.5 mM-50 mM, optionally at a concentration of about 25 mM; proline at a concentration of 12.5 mM-50 mM, optionally at a concentration of about 25 mM; asparagine at a concentration of 12.5 mM-50 mM, optionally at a concentration of about 25 mM; glycine at a concentration of 12.5 mM-50 mM, optionally at a concentration of about 25 mM; and arginine at a concentration of 12.5 mM-50 mM, optionally at a concentration of about 25 mM.

In some embodiments, the medium is for producing granulocyte colony-stimulating factor (G-CSF). In said embodiments, the one or more amino acids comprises one or more of glutamine, asparagine, aspartate, glutamate, arginine, histidine, leucine, and alanine. In some embodiments, the one or more amino acids comprises 2, 3, 4, 5, 6, 7, or all of glutamine, asparagine, aspartate, glutamate, arginine, histidine, leucine, and alanine. In some embodiments, the one or more amino acids comprises one or more of: glutamine at a concentration of 12.5 mM-50 mM, optionally at a concentration of about 25 mM; asparagine at a concentration of 12.5 mM-50 mM, optionally at a concentration of about 25 mM; aspartate at a concentration of 12.5 mM-50 mM, optionally at a concentration of about 25 mM; glutamate at a concentration of 12.5 mM-50 mM, optionally at a concentration of about 25 mM; arginine at a concentration of 12.5 mM-50 mM, optionally at a concentration of about 25 mM; histidine at a concentration of 12.5 mM-50 mM, optionally at a concentration of about 25 mM; leucine at a concentration of 12.5 mM-50 mM, optionally at a concentration of about 25 mM; and alanine at a concentration of 12.5 mM-50 mM, optionally at a concentration of about 25 mM. In some embodiments, the one or more amino acids comprises 2, 3, 4, 5, 6, 7, or all of: glutamine at a concentration of 12.5 mM-50 mM, optionally at a concentration of about 25 mM; asparagine at a concentration of 12.5 mM-50 mM, optionally at a concentration of about 25 mM; aspartate at a concentration of 12.5 mM-50 mM, optionally at a concentration of about 25 mM; glutamate at a concentration of 12.5 mM-50 mM, optionally at a concentration of about 25 mM; arginine at a concentration of 12.5 mM-50 mM, optionally at a concentration of about 25 mM; histidine at a concentration of 12.5 mM-50 mM, optionally at a concentration of about 25 mM; leucine at a concentration of 12.5 mM-50 mM, optionally at a concentration of about 25 mM; and alanine at a concentration of 12.5 mM-50 mM, optionally at a concentration of about 25 mM.

In some embodiments, the medium is for producing interferon alpha 2b (IFN). In said embodiments, the one or more amino acids comprises one or more of glutamine, arginine, asparagine, glutamate, isoleucine, aspartate, alanine, leucine, and histidine. In some embodiments, the one or more amino acids comprises 2, 3, 4, 5, 6, 7, 8, or all of glutamine, arginine, asparagine, glutamate, isoleucine, aspartate, alanine, leucine, and histidine. In some embodiments, the one or more amino acids comprises one or more of glutamine at a concentration of 12.5 mM-50 mM, optionally at a concentration of about 25 mM; arginine at a concentration of 12.5 mM-50 mM, optionally at a concentration of about 25 mM; asparagine at a concentration of 12.5 mM-50 mM, optionally at a concentration of about 25 mM; glutamate at a concentration of 12.5 mM-50 mM, optionally at a concentration of about 25 mM; isoleucine at a concentration of 12.5 mM-50 mM, optionally at a concentration of about 25 mM; aspartate at a concentration of 12.5 mM-50 mM, optionally at a concentration of about 25 mM; alanine at a concentration of 12.5 mM-50 mM, optionally at a concentration of about 25 mM; leucine at a concentration of 12.5 mM-50 mM, optionally at a concentration of about 25 mM; and histidine at a concentration of 12.5 mM-50 mM, optionally at a concentration of about 25 mM. In some embodiments, the one or more amino acids comprises 2, 3, 4, 5, 6, 7, 8, or all of glutamine at a concentration of 12.5 mM-50 mM, optionally at a concentration of about 25 mM; arginine at a concentration of 12.5 mM-50 mM, optionally at a concentration of about 25 mM; asparagine at a concentration of 12.5 mM-50 mM, optionally at a concentration of about 25 mM; glutamate at a concentration of 12.5 mM-50 mM, optionally at a concentration of about 25 mM; isoleucine at a concentration of 12.5 mM-50 mM, optionally at a concentration of about 25 mM; aspartate at a concentration of 12.5 mM-50 mM, optionally at a concentration of about 25 mM; alanine at a concentration of 12.5 mM-50 mM, optionally at a concentration of about 25 mM; leucine at a concentration of 12.5 mM-50 mM, optionally at a concentration of about 25 mM; and histidine at a concentration of 12.5 mM-50 mM, optionally at a concentration of about 25 mM.

In some embodiments, the media comprises glutamine at a concentration of ≤15 g/L, wherein the g/L denote concentration when the media is diluted in a solvent.

In some embodiments, the media comprises arginine at a concentration of ≤15 g/L, wherein the g/L denote concentration when the media is diluted in a solvent.

In some embodiments, the media comprises 2, 3, 4, 5, 6 or all of biotin, calcium pantothenate, nicotinic acid, inositol, thiamin HCl, pyridoxine HCl, and para-aminobenzoic acid. In certain embodiments, the media comprises one or more of: biotin at a concentration of 0.01-1.0 mg/L, optionally at a concentration of about 0.5 mg/L; calcium pantothenate at a concentration of 0.1-5 mg/L, optionally at a concentration of about 1.0 mg/L; nicotinic acid at a concentration of 0.1-5 mg/L, optionally at a concentration of about 1.0 mg/L; inositol at a concentration of 10-50 mg/L, optionally at a concentration of about 25 mg/L; thiamin HCl at a concentration of 0.1-5 mg/L, optionally at a concentration of about 1.0 mg/L; pyridoxine HCl at a concentration of 0.1-5 mg/L, optionally at a concentration of about 1.0 mg/L; and para-aminobenzoic acid at a concentration of 0.05-0.5 mg/L, optionally about 0.2 mg; wherein the g/L denote concentration when the media is diluted in a solvent. In some embodiments, the media comprises 2, 3, 4, 5, 6 or all of: biotin at a concentration of 0.01-1.0 mg/L, optionally at a concentration of about 0.5 mg/L; calcium pantothenate at a concentration of 0.1-5 mg/L, optionally at a concentration of about 1.0 mg/L; nicotinic acid at a concentration of 0.1-5 mg/L, optionally at a concentration of about 1.0 mg/L; inositol at a concentration of 10-50 mg/L, optionally at a concentration of about 25 mg/L; thiamin HCl at a concentration of 0.1-5 mg/L, optionally at a concentration of about 1.0 mg/L; pyridoxine HCl at a concentration of 0.1-5 mg/L, optionally at a concentration of about 1.0 mg/L; and para-aminobenzoic acid at a concentration of 0.05-0.5 mg/L, optionally about 0.2 mg; wherein the g/L denote concentration when the media is diluted in a solvent.

In some embodiments, the KH₂PO₄ is present at a concentration of 10-15 g/L, optionally about 12 g/L, wherein the g/L denote concentration when the media is diluted in a solvent.

In some embodiments, the MgSO₄ is present at a concentration of 3-7 g/L MgSO₄.7H₂O, optionally about 4.7 g/L MgSO₄.7H₂O, wherein the g/L denote concentration when the media is diluted in a solvent.

In some embodiments, the CaCl₂ is present at a concentration of 0.3-0.4 g/L CaCl₂.2H₂O, optionally about 0.36 g/L CaCl₂.2H₂O, wherein the g/L denote concentration when the media is diluted in a solvent.

In some embodiments, the media further comprises one or more of copper, iodine, manganese, molybdenum, boron, cobalt, zinc, iron, and vanadate. In certain embodiments, the media further comprises 2, 3, 4, 5, 6, 7, 8, or all of copper, iodine, manganese, molybdenum, boron, cobalt, zinc, iron, and vanadate. In some embodiments, the media further comprises one or more of: copper at a concentration of 5-10 mg/L, optionally at a concentration of about 6.64 mg/L; iodine at a concentration of 0.05-1.0 mg/L, optionally at a concentration of about 0.29 mg/L; manganese at a concentration of 2-8 mg/L, optionally at a concentration of about 4.24 mg/L; molybdenum at a concentration of 0.05-1.0 mg/L, optionally at a concentration of about 0.35 mg/L; boron at a concentration of 0.005-0.1 mg/L, optionally at a concentration of about 0.02 mg/L; cobalt at a concentration of 0.1-5.0 mg/L, optionally at a concentration of about 0.99 mg/L; zinc at a concentration of 5-100 mg/L, optionally at a concentration of about 41.47 mg/L; and iron at a concentration of 5-100 mg/L, optionally at a concentration of about 56.80 mg/L, wherein the g/L denote concentration when the media is diluted in a solvent. In some embodiments, the media further comprises 2, 3, 4, 5, 6, 7, or all of: copper at a concentration of 5-10 mg/L, optionally at a concentration of about 6.64 mg/L; iodine at a concentration of 0.05-1.0 mg/L, optionally at a concentration of about 0.29 mg/L; manganese at a concentration of 2-8 mg/L, optionally at a concentration of about 4.24 mg/L; molybdenum at a concentration of 0.05-1.0 mg/L, optionally at a concentration of about 0.35 mg/L; boron at a concentration of 0.005-0.1 mg/L, optionally at a concentration of about 0.02 mg/L; cobalt at a concentration of 0.1-5.0 mg/L, optionally at a concentration of about 0.99 mg/L; zinc at a concentration of 5-100 mg/L, optionally at a concentration of about 41.47 mg/L; and iron at a concentration of 5-100 mg/L, optionally at a concentration of about 56.80 mg/L, wherein the g/L denote concentration when the media is diluted in a solvent.

In some embodiments, the media is in a liquid form. In certain embodiments, the media is in a powder form for reconstitution in a liquid. In further embodiments, the media further comprises instructions for reconstituting the media. In some embodiments, the media is supplied as a concentrated liquid for dilution in a liquid.

In some embodiments, one or more components of the media are provided separately from the remainder of the media. In certain embodiments, the one or more of arachidonic fatty acid, linoleic fatty acid, linolenic fatty acid, myristic fatty acid, oleic fatty acid, palmitic fatty acid, stearic fatty acid, and cholesterol are provided separately from the remainder of the media. In some embodiments, the one or more of biotin, calcium pantothenate, nicotinic acid, inositol, thiamin HCl, pyridoxine HCl, and para-aminobenzoic acid are provided separately from the remainder of the media. In some embodiments, arginine and/or glutamine are provided separately from the remainder of the media.

In some embodiments, the pH of the media is 3-8. In certain embodiments, the pH of the media is 5-7.5. In further embodiments, the pH of the media is 6.5.

In another aspect, provided herein are methods of making a cell culture media. The method comprises dissolving a powdered media described herein in a liquid. In certain embodiments, the methods further comprise adjusting the pH of the media.

In some embodiments, the methods further comprise adding one or more components of the media that are provided separately from the remainder of the media to the powdered media. In certain embodiments, the one or more of arachidonic fatty acid, linoleic fatty acid, linolenic fatty acid, myristic fatty acid, oleic fatty acid, palmitic fatty acid, stearic fatty acid, and cholesterol are provided separately from the remainder of the media. In some embodiments, the one or more of biotin, calcium pantothenate, nicotinic acid, inositol, thiamin HCl, pyridoxine HCl, and para-aminobenzoic acid are provided separately from the remainder of the media. In some embodiments, arginine and/or glutamine are provided separately from the remainder of the media.

In another aspect, provided herein are cell cultures comprising the media described herein and one or more cells. In some embodiments, the cells are yeast cells. In certain embodiments, the yeast cells are Pichia pastoris cells.

In another aspect, provided herein are methods of culturing cells. The methods comprise introducing a population of cells into the media described herein; and growing the population of cells.

In some embodiments, the population of cells comprises yeast cells. In some embodiments, the yeast cells are Pichia pastoris cells.

In some embodiments, the media is supplemented with a first carbon source. In some embodiments, the methods further comprise providing media with a second carbon source. In some embodiments, the first and/or second carbon source is selected from methanol, glycerol, sorbitol, glucose, galactose, raffinose, sucrose, trehalose, lactic acid, ethanol, oleic acid, xylose, xylitol, inulin, gluconate, fructose, arabinose, corn syrup, corn steep liquor, mannose, lactose maltitol, ribose, melibiose, maltose, inulin, inositol, sorbose, arabitol, ribitol, myo-inositol, glucono-1,5-lactone, lactate, quinic acid, and gluconate. In some embodiments the first and/or second carbon source is another non-fermentable carbon source not provided herein.

In some embodiments, the population of cells is grown in a shake flask. In certain embodiments, the population of cells is grown in a batch bioreactor. In some embodiments, the population of cells is grown in a fed batch bioreactor. In certain embodiments, the population of cells is grown in a continuous flow bioreactor. In some embodiments, the population of cells is grown in a perfusion bioreactor. In certain embodiments, the population of cells is grown in a chemostat bioreactor.

In some embodiments, the population of cells is of a number of cells such that the ammonium does not increase above 5.5 g/g cells (dry weight).

In another aspect, provided herein are kits for culturing cells. The kits comprise the media described herein and means suitable for culturing the cells.

In some embodiments, the cells are yeast cells. In some embodiments, the yeast cells are Pichia pastoris cells.

In some embodiments, the kits further comprise instructions for culturing the cells.

In another aspect, provided herein are methods for improving cell growth or maintenance in vitro. The methods comprise culturing a first cell population under a first condition, obtaining and analyzing expression products from an aliquot of the cultured cell population, identifying expression products having an altered expression level relative to a control, and modifying the first culture condition by addition and/or deletion of one or more culture components based on the identity of the expression products having an altered expression level.

In some embodiments, the expression products having an altered expression level are involved in cellular metabolism. In certain embodiments, the expression products having an altered expression level are involved in fatty acid synthesis. In some embodiments, the expression products having an altered expression level are involved in vitamin synthesis. In certain embodiments, the expression products having an altered expression level are involved in amino acid synthesis. In some embodiments, the expression products having an altered expression level are involved in nucleotide synthesis. In certain embodiments, the expression products having an altered expression level are involved in protein glycosylation. In some embodiments, the expression products having an altered expression level are involved in redox biochemistry. In certain embodiments, the expression products having an altered expression level are involved in electron transport.

In some embodiments, the control is expression products from a first cell population cultured under a second condition that is different from the first condition. In certain embodiments, the first condition comprises a minimal culture medium and the second condition comprises a complex culture medium.

In some embodiments, the control is expression products from a second cell population cultured under the first condition, wherein the first and second cell populations are different. In certain embodiments, the first and second cell populations are different strains of a microorganism. In some embodiments, the first and second cell populations are different microorganisms.

In some embodiments, the first cell population is a Pichia pastoris strain. In certain embodiments, the first and second cell populations are different Pichia pastoris strains.

In some embodiments, the expression products having an altered expression level are reporter metabolites as defined by a genome-scale model.

In some embodiments, the first culture condition is modified by addition of one or more culture components based on the identity of the expression products having an altered expression level. In certain embodiments, the first culture condition is modified by increasing the concentration of one or more culture components based on the identity of the expression products having an altered expression level. In some embodiments, the first culture condition is modified by deletion of one or more culture components based on the identity of the expression products having an altered expression level. In certain embodiments, the first culture condition is modified by decreasing the concentration of one or more culture components based on the identity of the expression products having an altered expression level.

In some embodiments, the one more culture components comprise one or more lipids. In certain embodiments, the one or more culture components comprise one or more vitamins. In some embodiments, the one or more culture components comprise one or more amino acids.

In some embodiments, expression products having an altered expression level are defined as expression products having a log 2 fold change that is greater than 2 with an adjusted p-value of less than 0.05.

### BRIEF DESCRIPTION OF DRAWINGS

Non-limiting embodiments of the present invention will be described by way of example with reference to the accompanying Figures, which are schematic and are not intended to be drawn to scale. In the Figures:

FIG. 1 is a graph showing growth (OD600) over time (hr) in complex medium Buffered Glycerol Complex Medium (BMGY) at pH 6.5 (top, light blue), minimal medium Basal Salt Medium (BSM) at pH 6.5 (middle, grey), BSM at pH 5 media (middle, orange) and BMGY and BSM (minimal and complex) combined media (middle, yellow);

FIG. 2A is a graph showing Pichia growth (OD600) in 0.25M and 0.50M NH₄⁺;

FIG. 2B is a graph showing Pichia growth rate (h⁻¹) at the NH₄⁺ concentrations shown;

FIG. 3 is a graph showing Pichia growth rate (OD600) in BSM, FM22, and d'Anjou media each modified to have 25 mM NH₄⁺ and in BMGY media having a NH₄⁺ concentration of 150 mM;

FIG. 4A is a graph showing Pichia growth rate (OD600) in BMGY (top, light blue), BSM (lower, orange), d'Anjou (lower, grey), or Generation 1 media (middle, yellow) (25 mM NH₄⁺, 5 mM glutamine, 5 mM arginine, and the vitamins solution) media;

FIG. 4B is a graph showing ammonium (g/L) concentration over time in Generation 1 media;

FIG. 4C is a graph showing amino acid concentration over time in Generation 1 media;

FIG. 5 is a graph showing Pichia growth (OD600) over time (hr) in BMGY media (middle, light blue), Generation 1 media (bottom, yellow), d'Anjou media having 25 mM NH₄ and 5 mM arginine and glutamine and vitamins and 10 ml/L lipids (denoted +fatty acids) (middle, green), d'Anjou media having 25 mM NH₄ and 10 mM arginine and glutamine (denoted 2×AA's) and vitamins (middle, grey), or d'Anjou media having 25 mM NH₄ and 10 ml/L lipids and 10 mM arginine and glutamine and vitamins (Generation 2 media, denoted +lipids, 2×AA's) (top, dark blue);

FIG. 6A is a table showing Pichia growth (OD600) in BMGY, BSM, or Generation 2 media with a media exchange at 24 hours from glycerol-containing Generation 2 media to methanol-containing Generation 2 media; and

FIG. 6B is an SDS-PAGE gel showing human growth hormone (hGH) concentration generated by the cells in FIG. 6A after 48 hours of culture in BMGY, BSM or Generation 2 medium.

FIG. 7 shows differences between complex and defined cultivation media.

FIG. 8 shows growth curves comparing P. pastoris grown in buffered glycerol complex medium (BMGY) and a rich defined medium (RDM).

FIG. 9 shows SDS-PAGE gels of G-CSF expression by P. pastoris grown in BMGY and RDM. Protein supernatant samples were taken 24 hours after methanol induction.

FIG. 10 shows the expression of three recombinant proteins, hGH, G-CSF, and IFN, by P. pastoris grown in media with different individual amino acids at 12.5 mM each. Protein concentrations were measured by GXII, and samples were taken 24 hours after methanol induction.

FIG. 11 shows hGH expression by P. pastoris grown in media with different combinations of amino acids, each 12.5 mM total, based on initial individual amino acid screenings. hGH concentrations were measured by GXII, and samples were taken 24 hours after methanol induction.

FIG. 12 shows hGH produced in media with various amino acid compositions run on an SDS-PAGE gel. The standard is 0.1 mg/mL hGH and 0.1 mg/mL GCSF. In all media conditions, total amino acid concentration was normalized to 25 mM. The starred amino acid conditions are media formulations of interest.

FIG. 13 shows the amount of hGH produced in the amino acids shown at a total concentration of 12.5, 25, or 50 mM. The concentrations were measured by ELISA, and each value is the average of three technical replicates.

FIGS. 14A-14B show fermentation data for perfusion cultivations. FIG. 14A: Illustrative dissolved oxygen traces from the cultivation. FIG. 14B: Biomass and heterologous protein titer over the course of the cultivation. Biomass was measured by wet cell weight. hGH titers were measured by RPLC. The vertical lines show the time points used for RNA sampling.

FIGS. 15A-15C shows fermentation data for perfusion cultivations, analyzed separately for reactors with different induction times relative to the DO spike. FIG. 15A: Biomass measured by wet cell weight. FIG. 15B: hGH titer measured by RPLC. FIG. 15C: Hierarchical clustering of gene expression across all reactors and sample times.

FIGS. 16A-16E show a comparison of gene expression at different time points of the perfusion cultivation. The axes show the log 2(fpkm) values, where fpkm is the fragments per kilobase of transcript. FIG. 16A: G1 versus G2. FIG. 16B: G1 versus G3. FIG. 16C: G1 versus M1. FIG. 16D: G1 versus M2. FIG. 16E: G1 versus M3. G1, G2, G3, M1, M2 and M3 as defined in FIG. 14B.

FIG. 17 shows gene expression analyzed based on biological processes. The heat map shows row-normalized ssGSEA scores, averaged across the replicates for each condition. Conditions G1, G2, M2, and M3 have 7 replicates each. Conditions G3 and M1 have 3 replicates each.

FIG. 18 shows gene expression over time grouped by biological process. The heat map shows row-normalized ssGSEA scores, averaged across the replicates for each condition. Conditions G1, G2, M2, and M3 have 7 replicates each. Conditions G3 and M1 have 3 replicates each.

FIG. 19 shows a subset of the gene expression data from FIG. 17 demonstrating that when glycerol is limited, signs of nutrient limitation are evident.

FIG. 20 shows a subset of the gene expression data from FIG. 17 demonstrating that signs of nutrient limitation persist during methanol feeding.

FIG. 21 shows a subset of the gene expression data from FIG. 17 demonstrating that cell productivity declines during methanol feeding.

FIGS. 22A-22B show gene expression over time for specific genes of interest. FIG. 22A: Expression of hGH. FIG. 22B: Expression of genes involved in protein folding and secretion. The heat map shows the log 2(fpkm) values, where fpkm is the fragments per kilobase of transcript. Conditions G1, G2, M2, and M3 have 7 replicates each. Conditions G3 and M1 have 3 replicates each.

FIGS. 23A-23D show fermentation data for perfusion cultivations at higher methanol feed rates. The samples 15%-1 and 15%-2 are replicates. FIG. 23A: Biomass measured by wet cell weight. FIG. 23B: hGH titer measured by gel electrophoresis. FIG. 23C: Cell-specific productivity, qp, calculated from biomass and titer values. FIG. 23D: Volumetric productivity, calculated from cell-specific productivity and biomass.

### DETAILED DESCRIPTION OF INVENTION

Provided herein are improved cultivation media for microorganisms including without limitation yeast, such as Pichia pastoris, and filamentous fungi. These media are particularly useful for the production of heterologous proteins from such microorganisms at least in part because it has been found, unexpectedly, to promote maximum growth of the host organism (e.g., as measured by optical density or colony forming units) and maximum heterologous protein production

The media described herein are improved chemically defined media. While complex media may have certain advantages in cultivation in some instances, such as heightened growth rate during the period of biomass accumulation, defined media is preferred for biomanufacturing to ensure batch-to-batch consistency and to simplify regulatory documentation. Another advantage of defined media is the ability to rigorously analyze its raw materials, thereby lowering the risk of introducing adventitious agents such as viruses into a media formulation. Standard defined media formulations for Pichia pastoris growth are minimal salt solutions from which the organism synthesizes all metabolic intermediates. Examples of such defined media include Basal Salt Medium (BSM), d'Anjou medium, and FM22 medium. The chemically defined media of this disclosure have been demonstrated to enhance growth rate and heterologous protein production relative to existing chemically defined media.

Relative to existing chemically defined media, the improved cultivation media described herein have one or more of the following properties and/or additional components: i) reduced ammonium levels; ii) one or more lipids; iii) glutamine; iv) arginine; v) asparagine; and/or vi) one or more vitamins. It has been found, in accordance with the invention, that supplementing a chemically defined minimal media with any one of these added components results in improved host cell growth and heterologous protein production. Even more surprisingly, combination of two or more or all of these components results in a medium that functions on par with if not better than complex media.

### Media Compositions

### Ammonium

It has been found in accordance with the disclosure that existing chemically defined media may contain nutrients at over 50× levels required for the first 24 hours of microorganism (e.g., yeast) culture. It has further been found that the levels of some of these nutrients appear to suppress growth or induce death of microorganisms in culture. One such nutrient is ammonium. As demonstrated in Example 1, host cells such as Pichia experienced a slower growth rate as ammonium levels increased. This was surprising at least in part because many chemically defined media used to grow yeast, such as but not limited to Pichia strains, have high ammonia levels. This negative effect on growth was specific to ammonium as it was found that changes in phosphate and sulfur levels had no significant effects on cell growth.

Thus, certain of the improved media of this disclosure comprise reduced ammonium levels relative to existing chemically defined media. Certain chemically defined media have ammonium levels of around 200 mM-500 mM. This can then be compounded by the addition of ammonia during culture in order to adjust pH. As shown in Example 1, the inventors surprisingly found that reducing ammonium levels in chemically defined media led to an increase in growth rate, e.g., an increase in biomass formation as shown by optical density measurement. It had previously been reported that ammonium levels impacted only the lag phase. The data provided herein establish that ammonium levels are also important for the growth (or exponential) phase of host cell culture.

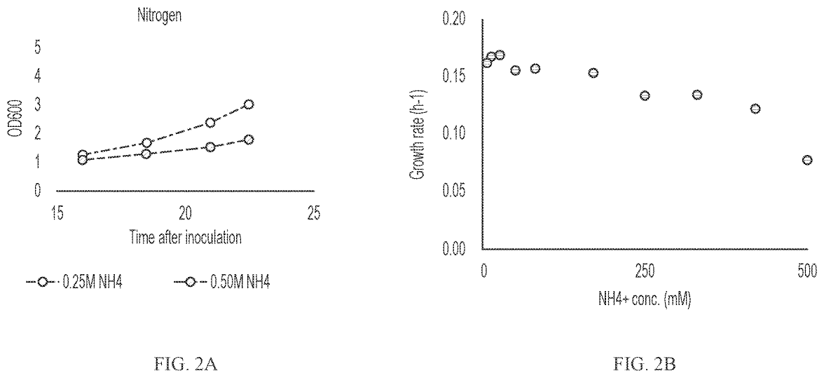

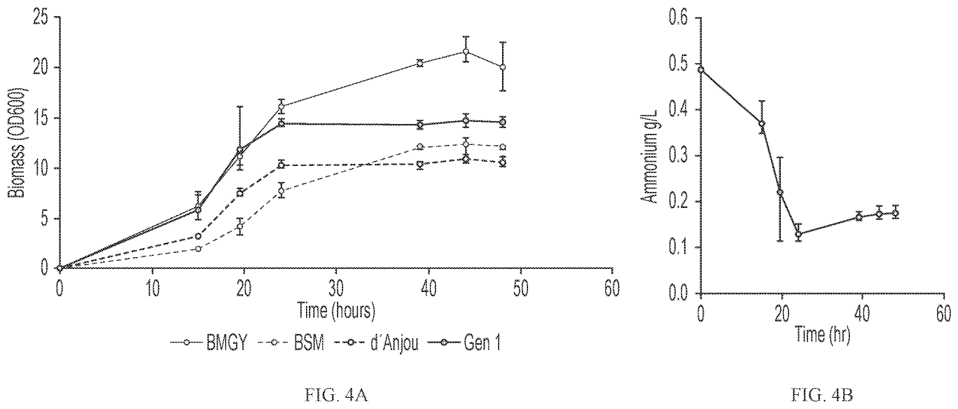

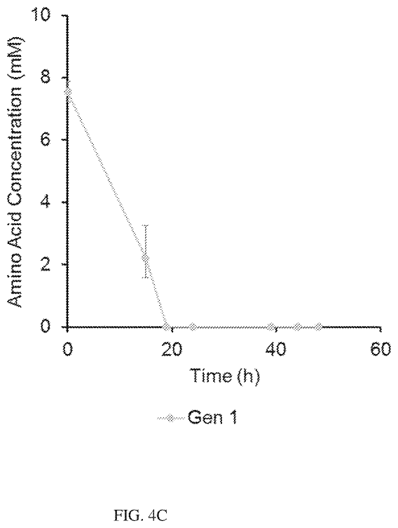

As shown in FIG. 2A, marked differences in biomass accumulation are apparent even between 0.25M and 0.5M ammonium at 20 hours after inoculation. FIG. 2B evidences the association between slower growth rate with increasing ammonium concentration. FIG. 4B further demonstrates that even an ammonium concentration on the order of 25 mM ammonium is sufficient for Pichia growth over a span of 50 hours.

As used herein, a reduced ammonium level intends an ammonium concentration that is less than 200 mM or less than 3.7 g/L. In some embodiments, the medium comprises less than 180 mM, 160 mM, 150 mM, 140 mM, 120 mM, 100 mM, 90 mM, 80 mM, 70 mM, 60 mM, 50 mM, 40 mM, 30 mM, or 20 mM ammonium.

In some embodiments, the medium comprises 10-20 mM, 10-30 mM, 10-50 mM, 10-80 mM, 10-100 mM, 10-150 mM, 10-200 mM, 15-20 mM, 15-30 mM, 15-50 mM, 15-80 mM, 15-100 mM, 15-150 mM, 15-200 mM, 20-40 mM, 20-60 mM, 20-100 mM, 50-70 mM, 50-100 mM, 50-150 mM, 50-200 mM, 100-120 mM, 100-150 mM, or 100-200 mM ammonium.

In some embodiments, the medium comprises about 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, or 200 mM ammonium.

In some embodiments, the medium comprises less than 3.5, 3.3, 3.0, 2.7, 2.5, 2.2, 2.0, 1.7, 1.5, 1.2, 1.0, 0.7, 0.5, 0.4, 0.3, 0.2, or 0.1 grams of ammonium per liter of solution.

In some embodiments, the medium comprises about 0.2-0.5, 0.2-1.0, 0.2-1.5, 0.2-2.0, 0.2-2.5, 0.2-3.0, 0.2-3.7, 0.5-1.0, 0.5-1.5, 1.0-1.5, 1.0-2.0, 1.5-2.0, 1.5-2.5, 2.0-3.0, 2.0-2.5, 2.5-3.0, or 3.0-3.7 grams of ammonium per liter of solution.

In some embodiments, the medium comprises about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, or 3.7 grams of ammonium per liter of solution.

In some embodiments, the level of ammonium is also dictated by the number of microorganisms in the culture. A lower concentration of ammonium may be used for growing a smaller number of cells, for example at the beginning of a culture, or a smaller biomass. Similarly, a higher concentration of ammonium may be used for growing a larger number of cells, for example at a later time point in the culture, or a larger biomass.

Thus, in some instances, the ammonium amount is also set at less than 5.5 grams of ammonium per gram of cells (dry weight). This may be represented, for example, as a culture having 5.5 grams of ammonium per liter of culture medium and 1 gram of cells (dry weight) per liter of culture medium. This level may also be expressed as an ammonium concentration that is less than 300 mM per gram cells (dry weight). This latter relationship may be represented, for example, as a culture having 300 millimoles of ammonium per liter of culture medium and 1 gram of cells (dry weight) per liter of culture medium.

When used in a culture, the medium or the number of cells may be adjusted to yield an ammonium level equal to or less than 5.5 g ammonium/g cells (dry weight), including without limitation less than about 5.5, 5.0, 4.5, 4.0, 3.5, 3.0, 2.5, 2.0, 1.5, 1.4, 1.3, 1.2, 1.1, 1.0, 0.9, 0.8, 0.7, 0.6, 0.5, 0.4, 0.3, 0.2, 0.1, 0.09, 0.08, 0.07, 0.06, 0.05, 0.04, or 0.03 g ammonium/g cells (dry weight).

The medium or the number of cells may be adjusted to yield an ammonium level equal to or less 0.02-5.5, 0.02-0.1, 0.02-0.5, 0.02-1.0, 0.05-0.1, 0.1-0.5, 0.1-1.0, 0.5-2.0, 1.0-2.0, 1.0-3.0, 1.5-4.0, 2.0-5.0, or 2.5-5.5 g ammonium/g cells (dry weight), or about 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, or 5.5 g ammonium/g cells (dry weight).

The medium or the number of cells may be adjusted to yield an ammonium concentration of less than about 300, 250, 200, 150, 100, 90, 80, 70, 60, 50, 40, 30, 20, 10, 9, 8, 7, 6, 5, 4, 3, or 2 mM ammonium/gram of cells (dry weight). In some embodiments, the medium or the number of cells may be adjusted to yield an ammonium concentration of about 1-300, 1-5, 1-10, 1-20, 1-20, 1-50, 1-100, 10-20, 10-50, 10-100, 50-100, 100-150, 100-200, 150-200, 150-250, 200-300, 250 and 300 mM ammonium/g cells (dry weight), or about 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 mM ammonium/g cells (dry weight).

Ammonium can be provided as an ammonium salt. Ammonium salts include, for example, CH₃CO₂NH₄, (NH₄)₂B₄O₇, NH₄Br, H₂NCO₂NH₄, (NH₄)₂CO₃, Ce(NH₄)₄(SO₄)₄, NH₄Cl, (NH₄)₂CrO₄, (NH₄)₂Cr₂O₇, NH₄H₂PO₄, NH₄F, HCO₂NH₄, (NH₄)₂TaF₇, (NH₄)₂TeBr₆, (NH₄)₃IrC₁₆, (NH₄)₂IrC₁₆, (NH₄)₂OsCl₆, (NH₄)₂PdCl₆, (NH₄)₂PtCl₆, (NH₄)₃RhCl₆, (NH₄)₂RuCl₆, (NH₄)₂TeCl₆, (NH₄)₂GeF₆, NH₄PF₆, (NH₄)₂SiF₆, (NH₄)₂SnF₆, (NH₄)₂TiF₆, NH₄HF₂, (NH₄)HC₂O₄, (NH₄)HSO₄, NH₄I, (NH₄)₆H₂W₁₂O₄₀, NH₄VO₃, (NH₄)₂MoO₄, NH₄NO₃, (NH₄)₂C₂O₄, (NH₄)₁₀H₂(W₂O₇)₆, (NH₄)B₅O₈, NH₄ReO₄, (NH₄)₂HPO₄, (NH₄)₃PMo₁₂O₄₀, NaNH₄HPO₄, (NH₄)₂SO₄, NH₄AuCl₄, (NH₄)₂PdCl₄, (NH₄)₂PtCl₄, NH₄BF₄, (NH₄)₂MoS₄, (NH₄)₂WS₄, (NH₄)₂S₂O₃, (NH₄)₂TiO(C₂O₄)₂, CF₃SO₃NH₄, H₈N₂PtS₁₅, (CH₃CH₂CH₂CH₂)₄N(IO₄), (CH₃CH₂CH₂CH₂)₄N(ReO₄), (C₂H₅)₄N(BF₄), and (CH₃)₄N(HCOO).

### Lipids

The inventors also found that existing chemically defined media could be dramatically improved by supplementation with one or more lipids. Surprisingly, the lipid requirement was revealed through an analysis of the transcriptome of Pichia cells cultured in minimal chemically defined media and in complex media. As discussed in greater detail below, such analysis revealed that Pichia altered its expression of genes regulating metabolites in lipid biosynthesis when grown in a modified chemically defined medium (Generation 1 media) relative to the complex media BMGY. More specifically, when cultured in the modified chemically defined medium, Pichia upregulated the expression of such genes as compared to Pichia cultured in complex media, suggesting that Pichia required such lipid biosynthesis for survival or growth.

This disclosure therefore provides improved media that comprise one or more exogenous lipids, optionally wherein such media are also defined as having reduced ammonium (as described above). Supplying Pichia cells with one or more exogenous lipids during culture ensures such lipids are present in non-limiting amounts, and results in cell growth not limited by lipid content.

According to Example 1, when the chemically defined medium described herein is supplemented with lipids, the growth rate, e.g., the biomass as measured by optical density, increases relative to art known defined (d'Anjou) medium. The medium used in Example 1 also had reduced levels of ammonium. Accordingly, in some embodiments, the medium provided herein has reduced ammonium levels and comprises one or more lipids relative to existing media.

The one or more lipids include and may be selected from, for example, arachidonic fatty acid, linoleic fatty acid, linolenic fatty acid, myristic fatty acid, oleic fatty acid, palmitic fatty acid, stearic fatty acid, and cholesterol. In some embodiments, the medium described herein comprises 2, 3, 4, 5, 6, 7, or all of arachidonic fatty acid, linoleic fatty acid, linolenic fatty acid, myristic fatty acid, oleic fatty acid, palmitic fatty acid, stearic fatty acid, and cholesterol.

The media described herein may comprise arachidonic fatty acid or linoleic fatty acid or linolenic fatty acid or myristic fatty acid or oleic fatty acid or palmitic fatty acid or stearic fatty acid or cholesterol, or any combination thereof. For example, the media described herein may comprise arachidonic fatty acid and linoleic fatty acid, linolenic fatty acid, myristic fatty acid, oleic fatty acid, palmitic fatty acid, stearic fatty acid, or cholesterol. The media described herein may comprise linoleic fatty acid and linolenic fatty acid, myristic fatty acid, oleic fatty acid, palmitic fatty acid, stearic fatty acid, or cholesterol. The media described herein may comprise linolenic fatty acid and myristic fatty acid, oleic fatty acid, palmitic fatty acid, stearic fatty acid, or cholesterol. The media described herein may comprise myristic fatty acid and oleic fatty acid, palmitic fatty acid, stearic fatty acid, or cholesterol. The media described herein may comprise oleic fatty acid and palmitic fatty acid, stearic fatty acid, or cholesterol. The media described herein may comprise palmitic fatty acid and stearic fatty acid or cholesterol. The media described herein may comprise stearic fatty acid and cholesterol.

The media described herein may comprise any one of the lipid combinations shown in Table 1.

TABLE 1

| | arachi- | lino- | lino- | | | | | |
|---|---|---|---|---|---|---|---|---|
| | donic | leic | leic | myristic | oleic | palmitic | stearic | |
| | fatty | fatty | fatty | fatty | fatty | fatty | fatty | choles- |
| | acid | acid | acid | acid | acid | acid | acid | terol |
| 1 | x | | | | | | | |
| 2 | | x | | | | | | |
| 3 | | | x | | | | | |
| 4 | | | | x | | | | |
| 5 | | | | | x | | | |
| 6 | | | | | | x | | |
| 7 | | | | | | | x | |
| 8 | | | | | | | | x |
| 9 | x | x | | | | | | |
| 10 | x | | x | | | | | |
| 11 | x | | | x | | | | |
| 12 | x | | | | x | | | |
| 13 | x | | | | | x | | |
| 14 | x | | | | | | x | |
| 15 | x | | | | | | | x |
| 16 | | x | x | | | | | |
| 17 | | x | | x | | | | |
| 18 | | x | | | x | | | |
| 19 | | x | | | | x | | |
| 20 | | x | | | | | x | |
| 21 | | x | | | | | | x |
| 22 | | | x | x | | | | |
| 23 | | | x | | x | | | |
| 24 | | | x | | | x | | |
| 25 | | | x | | | | x | |
| 26 | | | x | | | | | x |
| 27 | | | | x | x | | | |
| 28 | | | | x | | x | | |
| 29 | | | | x | | | x | |
| 30 | | | | x | | | | x |
| 31 | | | | | x | x | | |
| 32 | | | | | x | | x | |
| 33 | | | | | x | | | x |
| 34 | | | | | | x | x | |
| 35 | | | | | | x | | x |
| 36 | | | | | | | x | x |
| 37 | x | x | x | | | | | |
| 38 | x | x | | x | | | | |
| 39 | x | x | | | x | | | |
| 40 | x | x | | | | x | | |
| 41 | x | x | | | | | x | |
| 42 | x | x | | | | | | x |
| 43 | x | | x | x | | | | |
| 44 | x | | x | | x | | | |
| 45 | x | | x | | | x | | |
| 46 | x | | x | | | | x | |
| 47 | x | | x | | | | | x |
| 48 | x | | | x | x | | | |
| 49 | x | | | x | | x | | |
| 50 | x | | | x | | | x | |
| 51 | x | | | x | | | | x |
| 52 | x | | | | x | x | | |
| 53 | x | | | | x | | x | |
| 54 | x | | | | x | | | x |
| 55 | x | | | | | x | x | |
| 56 | x | | | | | x | | x |
| 57 | x | | | | | | x | x |
| 58 | | x | x | x | | | | |
| 59 | | x | x | | x | | | |
| 60 | | x | x | | | x | | |
| 61 | | x | x | | | | x | |
| 62 | | x | x | | | | | x |
| 63 | | x | | x | x | | | |
| 64 | | x | | x | | x | | |
| 65 | | x | | x | | | x | |
| 66 | | x | | x | | | | x |
| 67 | | x | | | x | x | | |
| 68 | | x | | | x | | x | |
| 69 | | x | | | x | | | x |
| 70 | | x | | | | x | x | |
| 71 | | x | | | | x | | x |
| 72 | | x | | | | | x | x |
| 73 | | | x | x | x | | | |
| 74 | | | x | x | | x | | |
| 75 | | | x | x | | | x | |
| 76 | | | x | x | | | | x |
| 77 | | | x | | x | x | | |
| 78 | | | x | | x | | x | |
| 79 | | | x | | x | | | x |
| 80 | | | x | | | x | x | |
| 81 | | | x | | | x | | x |
| 82 | | | x | | | | x | x |
| 83 | | | | x | x | x | | |
| 84 | | | | x | x | | x | |
| 85 | | | | x | x | | | x |
| 86 | | | | x | | x | x | |
| 87 | | | | x | | x | | x |
| 88 | | | | x | | | x | x |
| 89 | | | | | x | x | x | |
| 90 | | | | | x | x | | x |
| 91 | | | | | x | | x | x |
| 92 | | | | | | x | x | x |
| 93 | x | x | x | x | | | | |
| 94 | x | x | x | | x | | | |
| 95 | x | x | x | | | x | | |
| 96 | x | x | x | | | | x | |
| 97 | x | x | x | | | | | x |
| 98 | x | x | | x | x | | | |
| 99 | x | x | | x | | x | | |
| 100 | x | x | | x | | | x | |
| 101 | x | x | | x | | | | x |
| 102 | x | x | | | x | x | | |
| 103 | x | x | | | x | | x | |
| 104 | x | x | | | x | | | x |
| 105 | x | x | | | | x | x | |
| 106 | x | x | | | | x | | x |
| 107 | x | x | | | | | x | x |
| 108 | x | | x | x | x | | | |
| 109 | x | | x | x | | x | | |
| 110 | x | | x | x | | | x | |
| 111 | x | | x | x | | | | x |
| 112 | x | | x | | x | x | | |
| 113 | x | | x | | x | | x | |
| 114 | x | | x | | x | | | x |
| 115 | x | | x | | | x | x | |
| 116 | x | | x | | | x | | x |
| 117 | x | | x | | | | x | x |
| 118 | x | | | x | x | x | | |
| 119 | x | | | x | x | | x | |
| 120 | x | | | x | x | | | x |
| 121 | x | | | x | | x | x | |
| 122 | x | | | x | | x | | x |
| 123 | x | | | x | | | x | x |
| 124 | x | | | | x | x | x | |
| 125 | x | | | | x | x | | x |
| 126 | x | | | | x | | x | x |
| 127 | x | | | | | x | x | x |
| 128 | | x | x | x | x | | | |
| 129 | | x | x | x | | x | | |
| 130 | | x | x | x | | | x | |
| 131 | | x | x | x | | | | x |
| 132 | | x | x | | x | x | | |
| 133 | | x | x | | x | | x | |
| 134 | | x | x | | x | | | x |
| 135 | | x | x | | | x | x | |
| 136 | | x | x | | | x | | x |
| 137 | | x | x | | | | x | x |
| 138 | | x | | x | x | x | | |
| 139 | | x | | x | x | | x | |
| 140 | | x | | x | x | | | x |
| 141 | | x | | x | | x | x | |
| 142 | | x | | x | | x | | x |
| 143 | | x | | x | | | x | x |
| 144 | | x | | | x | x | x | |
| 145 | | x | | | x | x | | x |
| 146 | | x | | | x | | x | x |
| 147 | | x | | | | x | x | x |
| 148 | | | x | x | x | x | | |
| 149 | | | x | x | x | | x | |
| 150 | | | x | x | x | | | x |
| 151 | | | x | x | | x | x | |
| 152 | | | x | x | | x | | x |
| 153 | | | x | x | | | x | x |
| 154 | | | x | | x | x | x | |
| 155 | | | x | | x | x | | x |
| 156 | | | x | | x | | x | x |
| 157 | | | x | | | x | x | x |
| 158 | | | | x | x | x | x | |
| 159 | | | | x | x | x | | x |
| 160 | | | | x | x | | x | x |
| 161 | | | | x | | x | x | x |
| 162 | | | | | x | x | x | x |
| 163 | x | x | x | x | x | | | |
| 164 | x | x | x | x | | x | | |
| 165 | x | x | x | x | | | x | |
| 166 | x | x | x | x | | | | x |
| 167 | x | x | x | | x | x | | |
| 168 | x | x | x | | x | | x | |
| 169 | x | x | x | | x | | | x |
| 170 | x | x | x | | | x | x | |
| 171 | x | x | x | | | x | | x |
| 172 | x | x | x | | | | x | x |
| 173 | x | x | | x | x | x | | |
| 174 | x | x | | x | x | | x | |
| 175 | x | x | | x | x | | | x |
| 176 | x | x | | | x | x | x | |
| 177 | x | x | | | x | x | | x |
| 178 | x | x | | | | x | x | x |
| 179 | x | | x | x | x | x | | |
| 180 | x | | x | x | x | | x | |
| 181 | x | | x | x | x | | | x |
| 182 | x | | x | | x | x | x | |
| 183 | x | | x | | x | x | | x |
| 184 | x | | x | | x | | x | x |
| 185 | x | | x | | | x | x | x |
| 186 | x | | | x | x | x | x | |
| 187 | x | | | x | x | x | | x |
| 188 | x | | | x | x | | x | x |
| 189 | x | | | x | | x | x | x |
| 190 | x | | | | x | x | x | x |
| 191 | | x | x | x | x | x | | |
| 192 | | x | x | x | x | | x | |
| 193 | | x | x | x | x | | | x |
| 194 | | x | x | x | | x | x | |
| 195 | | x | x | x | | x | | x |
| 196 | | x | x | x | | | x | x |
| 197 | | x | x | | x | x | x | |
| 198 | | x | x | | x | x | | x |
| 199 | | x | x | | | x | x | x |
| 200 | | x | | x | x | x | x | |
| 201 | | x | | x | x | x | | x |
| 202 | | x | | x | | x | x | x |
| 203 | | x | | | x | x | x | x |
| 204 | | | x | x | x | x | x | |
| 205 | | | x | x | x | x | | x |
| 206 | | | x | x | x | | x | x |
| 207 | | | x | x | | x | x | x |
| 208 | | | x | | x | x | x | x |
| 209 | | | | x | x | x | x | x |
| 210 | x | x | x | x | x | x | | |
| 211 | x | x | x | x | x | | x | |
| 212 | x | x | x | x | x | | | x |
| 213 | x | x | x | x | | x | x | |
| 214 | x | x | x | x | | x | | x |
| 215 | x | x | x | x | | | x | x |
| 216 | x | x | x | | x | x | x | |
| 217 | x | x | x | | x | x | | x |
| 218 | x | x | x | | | x | x | x |
| 219 | x | x | | x | x | x | x | |
| 220 | x | x | | x | x | x | | x |
| 221 | x | x | | | x | x | x | x |
| 222 | x | | x | x | x | x | x | |
| 223 | x | | x | x | x | x | | x |
| 224 | x | | x | x | x | | x | x |
| 225 | x | | x | x | | x | x | x |
| 226 | x | | x | | x | x | x | x |
| 227 | x | | | x | x | x | x | x |
| 228 | | x | x | x | x | x | x | |
| 229 | | x | x | x | x | x | | x |
| 230 | | x | x | x | x | | x | x |
| 231 | | x | x | x | | x | x | x |
| 232 | | x | x | | x | x | x | x |
| 233 | | x | | x | x | x | x | x |
| 234 | | | x | x | x | x | x | x |
| 235 | x | x | x | x | x | x | x | |
| 236 | x | x | x | x | x | x | | x |
| 237 | x | x | x | x | x | | x | x |
| 238 | x | x | x | x | | x | x | x |
| 239 | x | x | x | | x | x | x | x |
| 240 | x | x | | x | x | x | x | x |
| 241 | x | | x | x | x | x | x | x |
| 242 | | x | x | x | x | x | x | x |
| 243 | x | x | x | x | x | x | x | x |

In some embodiments, the medium comprises arachidonic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises arachidonic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises arachidonic fatty acid at a concentration of about 1-1,000, 2-200, 5-50, 10-30, 15-25, 16-24, 17-23, 18-22, or 19-21 μg/L. In some embodiments, the medium comprises arachidonic fatty acid at a concentration of about 50-1000, 100-500, 200-300, 240-260, or 245-255 mg/L. In some embodiments, the medium comprises arachidonic fatty acid at a concentration of about 20 μg/L. In some embodiments, the medium comprises arachidonic fatty acid at a concentration of about 250 mg/L.

In some embodiments, the medium comprises linoleic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, or 900 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises linoleic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises linoleic fatty acid at a concentration of about 0.1-10,000, 1-1,000, 10-500, 50-150, 80-120, 90-110, 95-105, 96-104, 97-103, 98-102, 99-101 μg/L. In some embodiments, the medium comprises linoleic fatty acid at a concentration of about 50-1000, 100-500, 200-300, 240-260, or 245-255 mg/L. In some embodiments, the medium comprises linoleic fatty acid at a concentration of about 100 μg/L. In some embodiments, the medium comprises linoleic fatty acid at a concentration of about 250 mg/L.

In some embodiments, the medium comprises linolenic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, or 900 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises linolenic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises linolenic fatty acid at a concentration of about 0.1-10,000, 1-1,000, 10-500, 50-150, 80-120, 90-110, 95-105, 96-104, 97-103, 98-102, 99-101 μg/L. In some embodiments, the medium comprises linolenic fatty acid at a concentration of about 50-1000, 100-500, 200-300, 240-260, or 245-255 mg/L. In some embodiments, the medium comprises linolenic fatty acid at a concentration of about 100 μg/L. In some embodiments, the medium comprises linolenic fatty acid at a concentration of about 250 mg/L.

In some embodiments, the medium comprises myristic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, or 900 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises myristic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises myristic fatty acid at a concentration of about 0.1-10,000, 1-1,000, 10-500, 50-150, 80-120, 90-110, 95-105, 96-104, 97-103, 98-102, 99-101 μg/L. In some embodiments, the medium comprises myristic fatty acid at a concentration of about 50-1000, 100-500, 200-300, 240-260, or 245-255 mg/L. In some embodiments, the medium comprises myristic fatty acid at a concentration of about 100 μg/L. In some embodiments, the medium comprises myristic fatty acid at a concentration of about 250 mg/L.

In some embodiments, the medium comprises oleic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, or 900 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises oleic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises oleic fatty acid at a concentration of about 0.1-10,000, 1-1,000, 10-500, 50-150, 80-120, 90-110, 95-105, 96-104, 97-103, 98-102, 99-101 μg/L. In some embodiments, the medium comprises oleic fatty acid at a concentration of about 50-1000, 100-500, 200-300, 240-260, or 245-255 mg/L. In some embodiments, the medium comprises oleic fatty acid at a concentration of about 100 μg/L. In some embodiments, the medium comprises oleic fatty acid at a concentration of about 250 mg/L.

In some embodiments, the medium comprises palmitic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, or 900 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises palmitic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises palmitic fatty acid at a concentration of about 0.1-10,000, 1-1,000, 10-500, 50-150, 80-120, 90-110, 95-105, 96-104, 97-103, 98-102, 99-101 μg/L. In some embodiments, the medium comprises palmitic fatty acid at a concentration of about 50-1000, 100-500, 200-300, 240-260, or 245-255 mg/L. In some embodiments, the medium comprises palmitic fatty acid at a concentration of about 100 μg/L. In some embodiments, the medium comprises palmitic fatty acid at a concentration of about 250 mg/L.

In some embodiments, the medium comprises stearic fatty acid at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, or 900 μg/L or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises stearic fatty acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, or 250 mg/L. In some embodiments, the medium comprises stearic fatty acid at a concentration of about 0.1-10,000, 1-1,000, 10-500, 50-150, 80-120, 90-110, 95-105, 96-104, 97-103, 98-102, 99-101 μg/L. In some embodiments, the medium comprises stearic fatty acid at a concentration of about 50-1000, 100-500, 200-300, 240-260, or 245-255 mg/L. In some embodiments, the medium comprises stearic fatty acid at a concentration of about 100 μg/L. In some embodiments, the medium comprises stearic fatty acid at a concentration of about 250 mg/L.

In some embodiments, the medium comprises cholesterol at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 200, 300, 400, or 500 g/L. In some embodiments, the medium comprises cholesterol at a concentration greater than about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, or 2.5 g/L. In some embodiments, the medium comprises cholesterol at a concentration of about 0.05-100, 0.1-50, 0.2-20, 0.5-5.0, 1.0-3.5, 1.5-3.0, 1.6-2.8, 1.7-2.7, 1.8-2.6, 1.9-2.5, 2.0-2.4, 2.1-2.3 g/L. In some embodiments, the medium comprises cholesterol at a concentration of about 2.2 g/L.

Additionally, the levels of one or more lipids may be varied according to the cell biomass. In some embodiments, a lower concentration of one or more lipids may be used for growing cells at a lower biomass, and conversely a higher concentration of one or more lipids may be used for growing cells at a higher biomass.

### Glutamine

It was further found, in accordance with this disclosure and using HPLC analysis, that complex media comprise a variety of amino acids, of which arginine, alanine and lysine were the most prevalent (Table 3). Nevertheless it was found surprisingly that glutamine and arginine were able to separately enhance cell growth, while alanine or lysine had little effect.

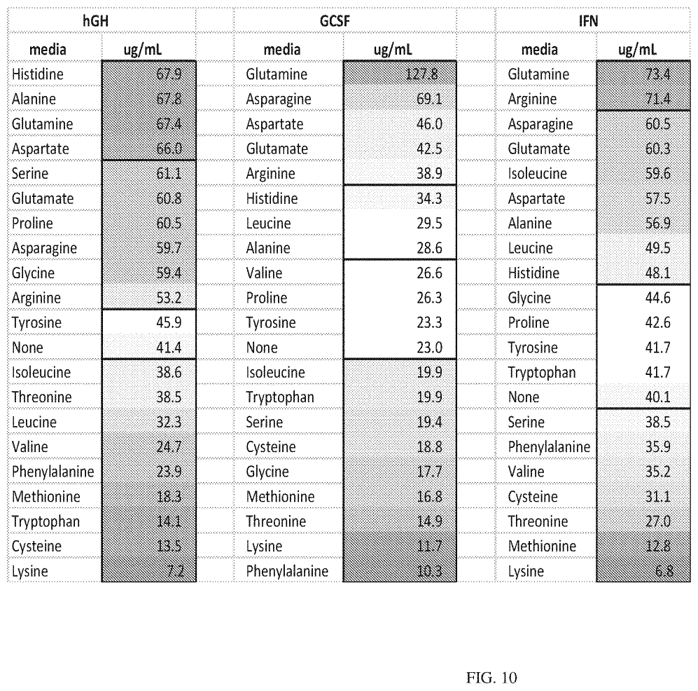

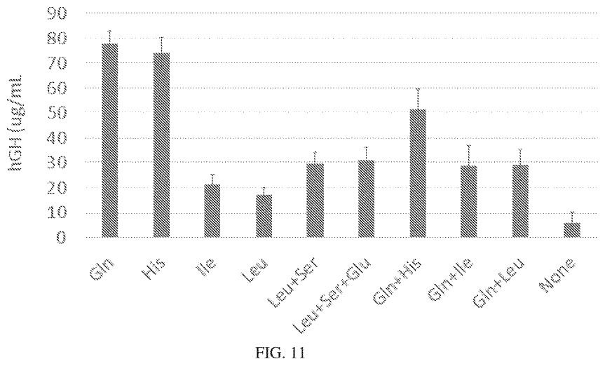

Specifically, the effect of glutamine on Pichia pastoris growth was evaluated, and the results are provided in Example 1. The inventors surprisingly found that supplementing chemically defined medium with glutamine led to the greatest increase in growth rate, e.g., an increase in biomass formation as shown by optical density measurement, compared to other amino acids tested (Table 4). Moreover, as is shown in Example 2, glutamine alone or with other amino acids increases protein production (see, e.g., FIG. 10, in which hGH, G-CSF, and IFN production is increased, and FIGS. 11-13, in which hGH production is increased). Thus, in some embodiments, the media provided herein are supplemented with glutamine. Optionally such media may further comprise reduced ammonium levels relative to existing complex media. Further presented in Example 1 are results demonstrating that growth rate, e.g., biomass as measured by optical density, is even further increased in medium with reduced ammonium levels and supplemented with lipids and glutamine. Accordingly, in some embodiments, the medium provided herein has reduced ammonium levels and is supplemented with glutamine and one or more lipids relative to existing complex media.

In some embodiments, the medium comprises glutamine at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15 g/L. In some embodiments, the medium comprises glutamine at a concentration less than about 15, 12, 10, 8, 6, 5, 4, 3, 2, 1, 0.5, 0.4, 0.3, 0.2, or 0.1 g/L. In some embodiments, the medium comprises glutamine at a concentration of about 0-15, 0-0.5, 0-1, 0-2, 0-5, 0-10, 2-4, 2-8, 2-10, 4-10, 6-10, 8-12, 10-15, or 12-15 g/L.

In some embodiments, the medium comprises glutamine at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises glutamine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises glutamine at a concentration of about 0.1-1, 1-2, 1-5, 1-10, 1-20, 1-50, 1-100, 5-20, 10-20, 10-50, 10-100, or 50-100 mM.

Additionally, the level of glutamine may be varied according to the cell biomass. In some embodiments, a lower concentration of glutamine may be used for growing cells at a lower biomass, and conversely a higher concentration of glutamine may be used for growing cells at a higher biomass.

In some embodiments, the medium comprises about 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15 g glutamine/g cells (dry weight). In some embodiments, the medium comprises less than about 15, 12, 10, 8, 6, 5, 4, 3, 2, 1, 0.5, 0.4, 0.3, or 0.2 g glutamine/g cells (dry weight). In some embodiments, the medium comprises about 0.15-15, 0.15-0.5, 0.15-1, 0.15-2, 0.15-5, 0.15-10, 2-4, 2-8, 2-10, 4-10, 6-10, 8-12, 10-15, or 12-15 g glutamine/g cells (dry weight).

In some embodiments, the medium comprises about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM glutamine/(g/L cells (dry weight)). In some embodiments, the medium comprises less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, or 2 mM glutamine/(g/L cells (dry weight)). In some embodiments, the medium comprises about 1-2, 1-5, 1-10, 1-20, 1-50, 1-100, 5-20, 10-20, 10-50, 10-100, or 50-100 mM glutamine/(g/L cells (dry weight)).

### Arginine

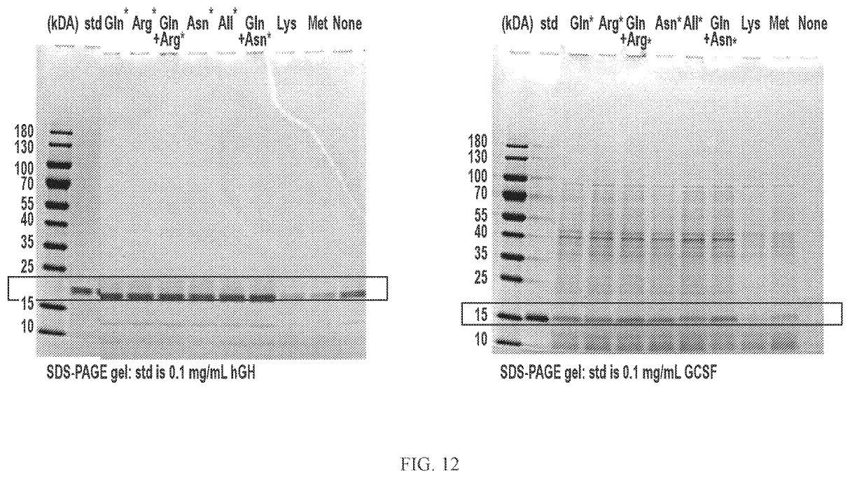

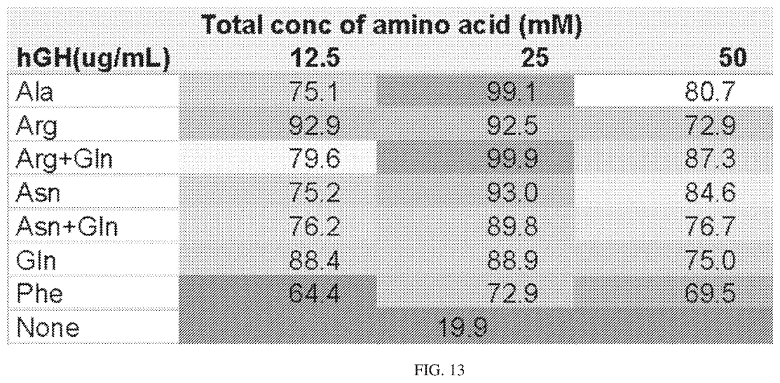

The other amino acid having positive effect on the growth of host cells was arginine. Moreover, as is shown in Example 2, arginine alone or with other amino acids increases protein production (see, e.g., FIG. 10, in which hGH, G-CSF, and IFN production is increased, and FIGS. 12-13, in which hGH production is increased). Thus, the media provided herein are supplemented with arginine. Similar to the finding with glutamine, it was found that arginine had a significant positive effect on host cell growth. The inventors found that supplementation of chemically defined media with arginine led to an increase in growth rate, e.g., an increase in biomass formation as shown by optical density measurement (Table 4). This result was surprising because a lesser effect was observed when alanine or lysine were used. This experiment was conducted using media having reduced ammonium. Therefore, in some embodiments, the medium provided herein has reduced ammonium levels and is supplemented with arginine relative to existing complex media. Further presented in Example 1 are results demonstrating that growth rate, e.g., biomass as measured by optical density, is even further increased in medium with reduced ammonium levels and supplemented with lipids, glutamine, and arginine. Accordingly, in some embodiments, the medium provided herein has reduced ammonium levels and is supplemented with glutamine, arginine, and one or more lipids relative to existing complex media.

In some embodiments, the medium comprises arginine at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15 g/L. In some embodiments, the medium comprises arginine at a concentration less than about 15, 12, 10, 8, 6, 5, 4, 3, 2, 1, 0.5, 0.4, 0.3, 0.2, or 0.1 g/L. In some embodiments, the medium comprises arginine at a concentration about 0-15, 0-0.5, 0-1, 0-2, 0-5, 0-10, 2-4, 2-8, 2-10, 4-10, 6-10, 8-12, 10-15, or 12-15 g/L.

In some embodiments, the medium comprises arginine at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises arginine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises arginine at a concentration of about 0.1-1, 1-2, 1-5, 1-10, 1-20, 1-50, 1-100, 5-20, 10-20, 10-50, 10-100, or 50-100 mM.

In some embodiments, the arginine levels were varied according to the cell biomass. In some embodiments, a lower concentration of arginine is provided for growing cells at a lower biomass. In some embodiments, a higher concentration of arginine is provided for growing cells at a higher biomass.

In some embodiments, the medium comprises about 0.15, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15 g arginine/g cells (dry weight). In some embodiments, the medium comprises less than about 15, 12, 10, 8, 6, 5, 4, 3, 2, 1, 0.5, 0.4, 0.3, or 0.2 g arginine/g cells (dry weight). In some embodiments, the medium comprises about 0.15-15, 0.15-0.5, 0.15-1, 0.15-2, 0.15-5, 0.15-10, 2-4, 2-8, 2-10, 4-10, 6-10, 8-12, 10-15, or 12-15 g arginine/g cells (dry weight).

In some embodiments, the medium comprises about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM arginine/(g/L cells (dry weight)). In some embodiments, the medium comprises less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, or 2 mM arginine/(g/L cells (dry weight)). In some embodiments, the medium comprises about 1-2, 1-5, 1-10, 1-20, 1-50, 1-100, 5-20, 10-20, 10-50, 10-100, or 50-100 mM arginine/(g/L cells (dry weight)).

### Asparagine

As is shown in Example 2, asparagine alone or with other amino acids increases protein production (see, e.g., FIG. 10, in which hGH, G-CSF, and IFN production is increased, and FIGS. 12-13, in which hGH production is increased). Thus, the media provided herein are supplemented with asparagine.

In some embodiments, the medium comprises asparagine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises asparagine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises asparagine at a concentration of about 0.1-100 or 12.5-50 mM, e.g., about 25 mM.

### Combinations of Amino Acids and Other Amino Acids

It was found, in accordance with this disclosure, that certain amino acids and combinations of amino acids increase protein expression. For example, FIG. 10 shows the effect of each of the 20 amino acids on hGH, G-CSF, and IFN expression. FIGS. 11-13 show the effect of individual and combinations of amino acids on hGH protein expression. As is shown in FIG. 11, while certain amino acids increase protein expression, other amino acids reduce protein expression to levels below that produced with no added amino acids.

In some embodiments, the medium described herein is supplemented with one or more of arginine, glutamine, asparagine, histidine, alanine, aspartate, serine, glutamate, proline, glycine, leucine, and isoleucine. In some embodiments, the medium described herein is supplemented with 2, 3, 4, 5, 6, 7, 8, 9, 10, 11, or all of arginine, glutamine, asparagine, histidine, alanine, aspartate, serine, glutamate, proline, glycine, leucine, and isoleucine.

In some embodiments, the medium described herein is supplemented with one or more of arginine, glutamine, and asparagine. In some embodiments, the medium described herein is supplemented with 2 or 3 of arginine, glutamine, and asparagine. In some embodiments, the medium is supplemented with arginine and glutamine. In some embodiments, the medium is supplemented with arginine and asparagine. In some embodiments, the medium is supplemented with glutamine and asparagine. In some embodiments, the medium is supplemented with arginine, glutamine, and asparagine.

It will be appreciated that certain amino acids may have a differing effect on protein production depending on the protein being produced. In some embodiments, the medium described herein is for producing human growth hormone and is supplemented with one or more of histidine, alanine, glutamine, aspartate, serine, glutamate, proline, asparagine, glycine, and arginine. It will be appreciated that certain amino acids may have a differing effect on protein production depending on the protein being produced. In some embodiments, the medium described herein is for producing human growth hormone and is supplemented with 2, 3, 4, 5, 6, 7, 8, 9, or all of histidine, alanine, glutamine, aspartate, serine, glutamate, proline, asparagine, glycine, and arginine. In some embodiments, the medium described herein is for producing G-CSF and is supplemented with one or more of glutamine, asparagine, aspartate, glutamate, arginine, histidine, leucine, and alanine. In some embodiments, the medium described herein is for producing G-CSF and is supplemented with 2, 3, 4, 5, 6, 7, or all of glutamine, asparagine, aspartate, glutamate, arginine, histidine, leucine, and alanine. In some embodiments, the medium described herein is for producing interferon alpha 2b and is supplemented with one or more of glutamine, arginine, asparagine, glutamate, isoleucine, aspartate, alanine, leucine, and histidine. In some embodiments, the medium described herein is for producing interferon alpha 2b and is supplemented with 2, 3, 4, 5, 6, 7, 8, or all of glutamine, arginine, asparagine, glutamate, isoleucine, aspartate, alanine, leucine, and histidine.

In some embodiments, the medium comprises histidine. In some embodiments, the medium comprises histidine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises histidine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises histidine at a concentration of about 0.1-100 or 12.5-50 mM, e.g., about 25 mM.

In some embodiments, the medium comprises alanine. In some embodiments, the medium comprises alanine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises alanine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises alanine at a concentration of about 0.1-100 or 12.5-50 mM, e.g., about 25 mM.

In some embodiments, the medium comprises aspartate. In some embodiments, the medium comprises aspartate at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises aspartate at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises aspartate at a concentration of about 0.1-100 or 12.5-50 mM, e.g., about 25 mM.

In some embodiments, the medium comprises serine. In some embodiments, the medium comprises serine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises serine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises serine at a concentration of about 0.1-100 or 12.5-50 mM, e.g., about 25 mM.

In some embodiments, the medium comprises glutamate. In some embodiments, the medium comprises glutamate at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises glutamate at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises glutamate at a concentration of about 0.1-100 or 12.5-50 mM, e.g., about 25 mM.

In some embodiments, the medium comprises proline. In some embodiments, the medium comprises proline at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises proline at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises proline at a concentration of about 0.1-100 or 12.5-50 mM, e.g., about 25 mM.

In some embodiments, the medium comprises glycine. In some embodiments, the medium comprises glycine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises glycine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises glycine at a concentration of about 0.1-100 or 12.5-50 mM, e.g., about 25 mM.

In some embodiments, the medium comprises leucine. In some embodiments, the medium comprises leucine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises leucine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises leucine at a concentration of about 0.1-100 or 12.5-50 mM, e.g., about 25 mM.

In some embodiments, the medium comprises isoleucine. In some embodiments, the medium comprises isoleucine at a concentration of about 1, 2, 3, 4, 5, 6, 7, 8, 9, 10, 11, 12, 13, 14, 15, 16, 17, 19, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the medium comprises isoleucine at a concentration less than about 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, or 1 mM. In some embodiments, the medium comprises isoleucine at a concentration of about 0.1-100 or 12.5-50 mM, e.g., about 25 mM.

### Vitamins

In some embodiments, the medium provided herein is supplemented with one or more vitamins. As is described in Example 1, the inventors found that supplementation of chemically defined medium with vitamins led to an increase in growth rate, e.g., an increase in biomass formation as shown by optical density measurement, particularly when combined with a reduced ammonium level. Accordingly, in some embodiments, the medium provided herein has reduced ammonium levels and is supplemented with vitamins relative to existing complex media. Further presented in Example 1 are results demonstrating that growth rate, e.g., biomass as measured by optical density, is even further increased in medium with reduced ammonium levels and supplemented with vitamins, glutamine, and arginine. Accordingly, in some embodiments, the medium provided herein has reduced ammonium levels and is supplemented with glutamine, arginine, and one or more vitamins relative to existing complex media.

In some embodiments, the medium described herein comprises one or more vitamins. The vitamins can include, for example, biotin, calcium pantothenate, nicotinic acid, inositol, thiamin HCl, pyridoxine HCl, and para-aminobenzoic acid. In some embodiments, the medium described herein comprises 2, 3, 4, 5, 6 or all of biotin, calcium pantothenate, nicotinic acid, inositol, thiamin HCl, pyridoxine HCl, and para-aminobenzoic acid.

The media may comprise biotin or calcium pantothenate or nicotinic acid or inositol or thiamin HCl or pyridoxine HCl or para-aminobenzoic acid. In some embodiments, the medium described herein comprises biotin and calcium pantothenate, or any combination thereof.

In some embodiments, the medium described herein comprises biotin and calcium pantothenate, nicotinic acid, inositol, thiamin HCl, pyridoxine HCl, or para-aminobenzoic acid. In some embodiments, the medium described herein comprises calcium pantothenate and nicotinic acid, inositol, thiamin HCl, pyridoxine HCl, or para-aminobenzoic acid. In some embodiments, the medium described herein comprises nicotinic acid and inositol, thiamin HCl, pyridoxine HCl, or para-aminobenzoic acid. In some embodiments, the medium described herein comprises inositol and thiamin HCl, pyridoxine HCl, or para-aminobenzoic acid. In some embodiments, the medium described herein comprises thiamin HCl and pyridoxine HCl, or para-aminobenzoic acid. In some embodiments, the medium described herein comprises pyridoxine HCl and para-aminobenzoic acid.

In some embodiments, the medium described herein comprises the combination of biotin, calcium pantothenate, nicotinic acid, inositol, thiamin HCl, pyridoxine HCl, and para-aminobenzoic acid shown in Table 2.

TABLE 2

| | | | | | | | Para- |
|---|---|---|---|---|---|---|---|
| | | Calcium | | | | Pyrido- | amino- |
| | | Panto- | Nicotinic | | Thiamin | xine | benzoic |
| | Biotin | thenate | Acid | Inositol | HCl | HCl | Acid |
| 1 | x | | | | | | |
| 2 | | x | | | | | |
| 3 | | | x | | | | |
| 4 | | | | x | | | |
| 5 | | | | | x | | |
| 6 | | | | | | x | |
| 7 | | | | | | | x |
| 8 | x | x | | | | | |
| 9 | x | | x | | | | |
| 10 | x | | | x | | | |
| 11 | x | | | | x | | |
| 12 | x | | | | | x | |
| 13 | x | | | | | | x |
| 14 | | x | x | | | | |
| 15 | | x | | x | | | |
| 16 | | x | | | x | | |
| 17 | | x | | | | x | |
| 18 | | x | | | | | x |
| 19 | | | x | x | | | |
| 20 | | | x | | x | | |
| 21 | | | x | | | x | |
| 22 | | | x | | | | x |
| 23 | | | | x | x | | |
| 24 | | | | x | | x | |
| 25 | | | | x | | | x |
| 26 | | | | | x | x | |
| 27 | | | | | x | | x |
| 28 | | | | | | x | x |
| 29 | x | x | x | | | | |
| 30 | x | x | | x | | | |
| 31 | x | x | | | x | | |
| 32 | x | x | | | | x | |
| 33 | x | x | | | | | x |
| 34 | x | | x | x | | | |
| 35 | x | | x | | x | | |
| 36 | x | | x | | | x | |
| 37 | x | | x | | | | x |
| 38 | x | | | x | x | | |
| 39 | x | | | x | | x | |
| 40 | x | | | x | | | x |
| 41 | x | | | | x | x | |
| 42 | x | | | | x | | x |
| 43 | x | | | | | x | x |
| 44 | | x | x | x | | | |
| 45 | | x | x | | x | | |
| 46 | | x | x | | | x | |
| 47 | | x | x | | | | x |
| 48 | | x | | x | x | | |
| 49 | | x | | x | | x | |
| 50 | | x | | x | | | x |
| 51 | | x | | | x | x | |
| 52 | | x | | | x | | x |
| 53 | | x | | | | x | x |
| 54 | | | x | x | x | | |
| 55 | | | x | x | | x | |
| 56 | | | x | x | | | x |
| 57 | | | x | | x | x | |
| 58 | | | x | | x | | x |
| 59 | | | x | | | x | x |
| 60 | | | | x | x | x | |
| 61 | | | | x | x | | x |
| 62 | | | | x | | x | x |
| 63 | | | | | x | x | x |
| 64 | x | x | x | x | | | |
| 65 | x | x | x | | x | | |
| 66 | x | x | x | | | x | |
| 67 | x | x | x | | | | x |
| 68 | x | x | | x | x | | |
| 69 | x | x | | x | | x | |
| 70 | x | x | | x | | | x |
| 71 | x | x | | | x | x | |
| 72 | x | x | | | x | | x |
| 73 | x | x | | | | x | x |
| 74 | x | | x | x | x | | |
| 75 | x | | x | x | | x | |
| 76 | x | | x | x | | | x |
| 77 | x | | x | | x | x | |
| 78 | x | | x | | x | | x |
| 79 | x | | x | | | x | x |
| 80 | x | | | x | x | x | |
| 81 | x | | | x | x | | x |
| 82 | x | | | x | | x | x |
| 83 | x | | | | x | x | x |
| 84 | | x | x | x | x | | |
| 85 | | x | x | x | | x | |
| 86 | | x | x | x | | | x |
| 87 | | x | x | | x | x | |
| 88 | | x | x | | x | | x |
| 89 | | x | x | | | x | x |
| 90 | | x | | x | x | x | |
| 91 | | x | | x | x | | x |
| 92 | | x | | x | | x | x |
| 93 | | x | | | x | x | x |
| 94 | | | x | x | x | x | |
| 95 | | | x | x | x | | x |
| 96 | | | x | x | | x | x |
| 97 | | | x | | x | x | x |
| 98 | | | | x | x | x | x |
| 99 | x | x | x | x | x | | |
| 100 | x | x | x | x | | x | |
| 101 | x | x | x | x | | | x |
| 102 | x | x | x | | x | x | |
| 103 | x | x | x | | x | | x |
| 104 | x | x | x | | | x | x |
| 105 | x | x | | x | x | x | |
| 106 | x | x | | x | x | | x |
| 107 | x | x | | | x | x | x |
| 108 | x | | x | x | x | x | |
| 109 | x | | x | x | x | | x |
| 110 | x | | x | x | | x | x |
| 111 | x | | x | | x | x | x |
| 112 | x | | | x | x | x | x |
| 113 | | x | x | x | x | x | |
| 114 | | x | x | x | x | | x |
| 115 | | x | x | x | | x | x |
| 116 | | x | x | | x | x | x |
| 117 | | x | | x | x | x | x |
| 118 | | | x | x | x | x | x |
| 119 | | x | x | x | x | x | x |
| 120 | x | | x | x | x | x | x |
| 121 | x | x | | x | x | x | x |
| 122 | x | x | x | | x | x | x |
| 123 | x | x | x | x | | x | x |
| 124 | x | x | x | x | x | | x |
| 125 | x | x | x | x | x | x | |
| 126 | x | x | x | x | x | x | x |

In some embodiments, the medium comprises biotin at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, or 100 mg/L. In some embodiments, the medium comprises biotin at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 1.0, or 1.5 mg/L. In some embodiments, the medium comprises biotin at a concentration of about 0.001-100, 0.01-10, 0.05-5, 0.09-2, 0.1-1.0, 0.2-0.8, 0.3-0.7, 0.4-0.6 mg/L. In some embodiments, the medium comprises biotin at a concentration of about 0.5 mg/L.

In some embodiments, the medium comprises calcium pantothenate at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 200, 300, 400, or 500 mg/L. In some embodiments, the medium comprises calcium pantothenate at a concentration greater than about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, or 1.5 mg/L. In some embodiments, the medium comprises calcium pantothenate at a concentration of about 0.001-100, 0.005-50, 0.01-10, 0.05-2.0, 0.1-1.0, 0.5-1.5, 0.6-1.4, 0.7-1.3, 0.8-1.2, 0.9-1.1 mg/L. In some embodiments, the medium comprises calcium pantothenate at a concentration of about 1.0 mg/L.

In some embodiments, the medium comprises nicotinic acid at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 200, 300, 400, or 500 mg/L. In some embodiments, the medium comprises nicotinic acid at a concentration greater than about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, or 1.5 mg/L. In some embodiments, the medium comprises nicotinic acid at a concentration about 0.001-100, 0.005-50, 0.01-10, 0.05-2.0, 0.1-1.0, 0.5-1.5, 0.6-1.4, 0.7-1.3, 0.8-1.2, 0.9-1.1 mg/L. In some embodiments, the medium comprises nicotinic acid at a concentration of about 1.0 mg/L.

In some embodiments, the medium comprises inositol at a concentration of about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 310, 350, 400, 450, 500, 600, 700, 800, 900 or 1,000 mg/L. In some embodiments, the medium comprises inositol at a concentration greater than about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, or 50 mg/L. In some embodiments, the medium comprises inositol at a concentration of about 0.1-1,000, 1-100, 5-50, 10-40, 15-35, 20-30, 21-29, 22-28, 23-27, or 24-26 mg/L. In some embodiments, the medium comprises inositol at a concentration of about 25 mg/L.

In some embodiments, the medium comprises thiamin HCl at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 200, 300, 400, or 500 mg/L. In some embodiments, the medium comprises thiamin HCl at a concentration greater than about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, or 1.5 mg/L. In some embodiments, the medium comprises thiamin HCl at a concentration of about 0.001-100, 0.005-50, 0.01-10, 0.05-2.0, 0.1-1.0, 0.5-1.5, 0.6-1.4, 0.7-1.3, 0.8-1.2, 0.9-1.1 mg/L. In some embodiments, the medium comprises thiamin HCl at a concentration of about 1.0 mg/L.

In some embodiments, the medium comprises pyridoxine HCl at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 200, 300, 400, or 500 mg/L. In some embodiments, the medium comprises pyridoxine HCl at a concentration greater than about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, or 1.5 mg/L. In some embodiments, the medium comprises pyridoxine HCl at a concentration of about 0.001-100, 0.005-50, 0.01-10, 0.05-2.0, 0.1-1.0, 0.5-1.5, 0.6-1.4, 0.7-1.3, 0.8-1.2, 0.9-1.1 mg/L. In some embodiments, the medium comprises pyridoxine HCl at a concentration of about 1.0 mg/L.

In some embodiments, the medium comprises para-aminobenzoic acid at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, or 100 mg/L. In some embodiments, the medium comprises para-aminobenzoic acid at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.5, or 1.0 mg/L. In some embodiments, the medium comprises para-aminobenzoic acid at a concentration of about 0.001-100, 0.005-50, 0.1-10, 0.02-2.0, 0.05-1.0, 0.06-0.7, 0.07-0.6, 0.08-0.5, 0.09-0.4, or 0.1-0.3 mg/L. In some embodiments, the medium comprises para-aminobenzoic acid at a concentration of about 0.2 mg/L.

The levels of one or more vitamins may be varied according to the cell biomass. In some embodiments, a lower concentration of one or more vitamins is provided for growing cells at a lower biomass. In some embodiments, a higher concentration of one or more vitamins is provided for growing cells at a higher biomass.

In some embodiments the medium described herein comprises reduced ammonium levels. In some embodiments the medium described herein comprises one or more lipids. In some embodiments the medium described herein comprises glutamine. In some embodiments the medium described herein comprises arginine. In some embodiments the medium described herein comprises asparagine. In some embodiments the medium described herein comprises one or more vitamins.

In some embodiments the medium described herein comprises reduced ammonium levels and comprises glutamine. In some embodiments the medium described herein comprises reduced ammonium levels and comprises one or more lipids. In some embodiments the medium described herein comprises reduced ammonium levels and comprises arginine. In some embodiments the medium described herein comprises reduced ammonium levels and comprises asparagine. In some embodiments the medium described herein comprises reduced ammonium levels and comprises one or more vitamins. In some embodiments the medium described herein comprises glutamine and one or more lipids. In some embodiments the medium described herein comprises glutamine and arginine. In some embodiments the medium described herein comprises glutamine and asparagine. In some embodiments the medium described herein comprises glutamine and one or more vitamins. In some embodiments the medium described herein comprises one or more lipids and arginine. In some embodiments the medium described herein comprises one or more lipids and asparagine. In some embodiments the medium described herein comprises one or more lipids and one or more vitamins. In some embodiments the medium described herein comprises arginine and one or more vitamins. In some embodiments the medium described herein comprises arginine and asparagine. In some embodiments the medium described herein comprises asparagine and one or more vitamins.

In some embodiments the medium described herein comprises reduced ammonium levels and comprises glutamine and one or more lipids. In some embodiments the medium described herein comprises reduced ammonium levels and comprises glutamine and arginine. In some embodiments the medium described herein comprises reduced ammonium levels and comprises glutamine and one or more vitamins. In some embodiments the medium described herein comprises reduced ammonium levels and comprises glutamine and asparagine. In some embodiments the medium described herein comprises reduced ammonium levels and comprises one or more lipids and arginine. In some embodiments the medium described herein comprises reduced ammonium levels and comprises one or more lipids and one or more vitamins. In some embodiments the medium described herein comprises reduced ammonium levels and comprises one or more lipids and asparagine. In some embodiments the medium described herein comprises reduced ammonium levels and comprises arginine and one or more vitamins. In some embodiments the medium described herein comprises reduced ammonium levels and comprises arginine and asparagine. In some embodiments the medium described herein comprises reduced ammonium levels and comprises asparagine and one or more vitamins. In some embodiments the medium described herein comprises glutamine, one or more lipids, and arginine. In some embodiments the medium described herein comprises glutamine, one or more lipids, and one or more vitamins. In some embodiments the medium described herein comprises glutamine, one or more lipids, and asparagine. In some embodiments the medium described herein comprises glutamine, arginine, and one or more vitamins. In some embodiments the medium described herein comprises glutamine, arginine, and asparagine. In some embodiments the medium described herein comprises glutamine, asparagine, and one or more vitamins. In some embodiments the medium described herein comprises one or more lipids, arginine, and one or more vitamins. In some embodiments the medium described herein comprises one or more lipids, arginine, and asparagine. In some embodiments the medium described herein comprises one or more lipids, asparagine, and one or more vitamins. In some embodiments the medium described herein comprises arginine, asparagine, and one or more vitamins.

In some embodiments the medium described herein comprises reduced ammonium levels and comprises glutamine, one or more lipids, and arginine. In some embodiments the medium described herein comprises reduced ammonium levels and comprises glutamine, one or more lipids, and one or more vitamins. In some embodiments the medium described herein comprises reduced ammonium levels and comprises glutamine, arginine, and one or more vitamins. In some embodiments the medium described herein comprises reduced ammonium levels and comprises one or more lipids, arginine, and one or more vitamins. In some embodiments the medium described herein comprises glutamine, one or more lipids, arginine, and one or more vitamins. In some embodiments the medium described herein comprises reduced ammonium levels and comprises glutamine, one or more lipids, and asparagine. In some embodiments the medium described herein comprises reduced ammonium levels and comprises glutamine, arginine, and asparagine. In some embodiments the medium described herein comprises reduced ammonium levels and comprises one or more lipids, arginine, and asparagine. In some embodiments the medium described herein comprises glutamine, one or more lipids, arginine, and asparagine. In some embodiments the medium described herein comprises glutamine, one or more lipids, one or more vitamins, and asparagine. In some embodiments the medium described herein comprises reduced ammonium levels and comprises one or more lipids, one or more vitamins, and asparagine. In some embodiments the medium described herein comprises reduced ammonium levels and comprises glutamine, one or more vitamins, and asparagine. In some embodiments the medium described herein comprises glutamine, arginine, one or more vitamins, and asparagine. In some embodiments the medium described herein comprises reduced ammonium levels and comprises arginine, one or more vitamins, and asparagine. In some embodiments the medium described herein comprises one or more lipids, arginine, one or more vitamins, and asparagine.

In some embodiments the medium described herein comprises reduced ammonium levels and comprises glutamine, one or more lipids, arginine, asparagine, and one or more vitamins.

### Salts and Trace Minerals

As is described above, the media described herein is a chemically defined media having one or more of the specified properties or additional components described herein. In some embodiments, the media described herein comprises components of a chemically defined media including, e.g., KH₂PO₄, MgSO₄, and CaCl₂.

In some embodiments, the medium described herein comprises KH₂PO₄. In some embodiments, the medium comprises KH₂PO₄ at a concentration of about 0.1, 0.5, 1, 2, 3, 4, 5, 6, 7, 8, 9, 10, 10, 11, 12, 13, 14, 15, 16, 17, 19, 19, 20, 21, 22, 23, 24, 25, 26, 27, 28, 29, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 350, 400, or 500 g/L. In some embodiments, the medium comprises KH₂PO₄ at a concentration greater than about 0.1, 0.5, 1, 2, 3, 4, 5, 6, 7, 8, 9, 10, 10, 11, 12, or 15 g/L. In some embodiments, the medium comprises KH₂PO₄ at a concentration of about 0.2-200, 1-50, 5-20, 9-17, 10-15, or 11-13 g/L. In some embodiments, the medium comprises KH₂PO₄ at a concentration of about 12 g/L.

In some embodiments, the medium described herein comprises MgSO₄. In some embodiments, the medium comprises MgSO₄.7H₂O at a concentration of about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 200, 300, 400, or 500 g/L. In some embodiments, the medium comprises MgSO₄.7H₂O at a concentration greater than about 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, or 5.0 g/L. In some embodiments, the medium comprises MgSO₄.7H₂O at a concentration of about 0.1-100, 0.5-20, 1-10, 2-8, 3-7, 4-6, 4.6-4.8 g/L. In some embodiments, the medium comprises MgSO₄.7H₂O at a concentration of about 4.7 g/L.

In some embodiments, the medium described herein comprises CaCl₂. In some embodiments, the medium comprises CaCl₂ at a concentration of about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, or 100 g/L. In some embodiments, the medium comprises CaCl₂ at a concentration greater than about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, or 1.0 g/L. In some embodiments, the medium comprises CaCl₂ at a concentration of about 0.001-100, 0.01-10, 0.05-5, 0.09-2, 0.1-1.0, 0.2-0.8, or 0.3-0.6 g/L. In some embodiments, the medium comprises CaCl₂ at a concentration of about 0.4 g/L.

In some embodiments, the medium described herein comprises KH₂PO₄ and MgSO₄. In some embodiments, the medium described herein comprises KH₂PO₄ and CaCl₂. In some embodiments, the medium described herein comprises MgSO₄ and CaCl₂. In some embodiments, the medium described herein comprises KH₂PO₄, MgSO₄, and CaCl₂.

In some embodiments, the medium described herein further comprises one or more trace minerals. Trace minerals in accordance with the invention can include copper, iodine, manganese, molybdenum, boron, cobalt, zinc, iron, and vanadate. In some embodiments, the medium comprises 2, 3, 4, 5, 6, 7, 8 or all of copper, iodine, manganese, molybdenum, boron, cobalt, zinc, iron, and vanadate.

In some embodiments, the medium comprises copper at a concentration of about 5, 5.5, 6, 6.5, 7, 7.5, 8, 8.5, 9, 9.5, or 10 mg/L. In some embodiments, the medium comprises copper at a concentration greater than about 5, 5.2, 5.4, 5.6, 5.8, 6, 6.2, 6.4, 6.6, 6.8, or 7 mg/L. In further embodiments, the medium comprises copper at a concentration of about 5-10, 5-9, 6-9, 6-8, or about 6-7 mg/L. In some embodiments, the medium comprises copper at a concentration of about 6.64 mg/L. In some embodiments, copper is provided as a salt. One exemplary salt is CuSO₄. Those of skill in the art will readily know of other salts that can be used, as this aspect of the invention is non-limiting. The salt is preferably pharmaceutically acceptable.

In some embodiments, the medium comprises iodine at a concentration of about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 mg/L. In some embodiments, the medium comprises iodine at a concentration greater than about 0.05, 0.1, 0.15, 0.2, 0.25, 0.3, or 0.35 mg/L. In other embodiments, the medium comprises iodine at a concentration of about 0.05-1.0, 0.05-0.5, 0.1-0.4, 0.15-0.35, 0.2-0.35, 0.2-0.3, or about 0.25-0.3 mg/L. In some embodiments, the medium comprises iodine at a concentration of about 0.29 mg/L. In some embodiments, iodine is provided as a salt. One exemplary salt is NaI. Those of skill in the art will readily know of other salts that can be used, as this aspect of the invention is non-limiting. The salt is preferably pharmaceutically acceptable.

In some embodiments, the medium comprises manganese at a concentration of about 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.5, 7, 7.5, or 8 mg/L. In some embodiments, the medium comprises manganese at a concentration greater than about 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, or 6 mg/L. In some embodiments, the medium comprises manganese at a concentration of about 2-8, 2.5-7.5, 3-7, 3.5-6.5, 4-6, or about 4-5 mg/L. In some embodiments, the medium comprises manganese at a concentration of about 4.24 mg/L. In some embodiments, manganese is provided as a salt. One exemplary salt is MnSO₄. Those of skill in the art will readily know of other salts that can be used, as this aspect of the invention is non-limiting. The salt is preferably pharmaceutically acceptable.

In some embodiments, the medium comprises molybdenum at a concentration of about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 mg/L. In some embodiments, the medium comprises molybdenum at a concentration greater than about 0.05, 0.1, 0.15, 0.2, 0.25, 0.3, 0.35, or 0.4 mg/L. In some embodiments, the medium comprises molybdenum at a concentration of about 0.05-1.0, 0.05-0.5, 0.1-0.4, 0.15-0.4, 0.2-0.4, or about 0.3-0.4 mg/L. In some embodiments, the medium comprises molybdenum at a concentration of about 0.35 mg/L. In some embodiments, molybdenum is provided as a salt. One exemplary salt is NaMoO₄. Those of skill in the art will readily know of other salts that can be used, as this aspect of the invention is non-limiting. The salt is preferably pharmaceutically acceptable.

In some embodiments, the medium comprises boron at a concentration of about 0.005, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, or 0.1 mg/L. In some embodiments, the medium comprises boron at a concentration greater than about 0.005, 0.01, 0.015, 0.02, 0.025 mg/L. In some embodiments, the medium comprises boron at a concentration of about 0.005-0.1, 0.01-0.09, 0.01-0.08, 0.01-0.07, 0.01-0.06, 0.01-0.05, 0.01-0.04, 0.01-0.03, or about 0.015-0.025. In some embodiments, the medium comprises boron at a concentration of about 0.02 mg/L. In some embodiments, boron is provided as a salt. One exemplary salt is BH₃O₃. Those of skill in the art will readily know of other salts that can be used, as this aspect of the invention is non-limiting. The salt is preferably pharmaceutically acceptable.

In some embodiments, the medium comprises cobalt at a concentration of about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 mg/L. In some embodiments, the medium comprises cobalt at a concentration greater than about 0.1, 0.2, 0.3, 0.4, 0.5, 0.6, 0.7, 0.8, 0.9, 1.0, 1.1, or about 1.2 mg/L. In some embodiments, the medium comprises cobalt at a concentration of about 0.1-5.0, 0.1-2.5, 0.5-2.0, 0.5-1.5, 0.8-1.0, or about 0.9-1.0 mg/L. In some embodiments, the medium comprises cobalt at a concentration of about 0.99 mg/L. In some embodiments, cobalt is provided as a salt. One exemplary salt is CoCl₂. Those of skill in the art will readily know of other salts that can be used, as this aspect of the invention is non-limiting. The salt is preferably pharmaceutically acceptable.

In some embodiments, the medium comprises zinc at a concentration of about 5, 6, 7, 8, 9, 10, 15, 20, 25, 30, 35, 40, 45, 50, 55, 60, 65, 70, 75, 80, 85, 90, 95, or 100 mg/L. In some embodiments, the medium comprises zinc at a concentration of greater than about 5, 6, 7, 8, 9, 10, 15, 20, 25, 30, 35, 40, 45, or 50 mg/L. In some embodiments, the medium comprises zinc at a concentration of about 5-100, 5-90, 10-90, 10-80, 20-80, 25-75, 30-70, 35-65, 35-60, 35-55, 35-50, 35-45, or about 37-43 mg/L. In some embodiments, the medium comprises zinc at a concentration of about 41.47 mg/L. In some embodiments, zinc is provided as a salt. One exemplary salt is ZnCl₂. Those of skill in the art will readily know of other salts that can be used, as this aspect of the invention is non-limiting. The salt is preferably pharmaceutically acceptable.

In some embodiments, the medium comprises iron at a concentration of about 5, 6, 7, 8, 9, 10, 15, 20, 25, 30, 35, 40, 45, 50, 55, 60, 65, 70, 75, 80, 85, 90, 95, or 100 mg/L. In some embodiments, the medium comprises iron at a concentration of greater than about 5, 6, 7, 8, 9, 10, 15, 20, 25, 30, 35, 40, 45, 50, 55, 60, or 65 mg/L. In some embodiments, the medium comprises iron at a concentration of about 5-100, 5-90, 10-90, 10-80, 20-80, 25-75, 30-70, 35-65, 40-60, 45-60, 50-60, or about 55-60 mg/L. In some embodiments, the medium comprises iron at a concentration of about 56.80 mg/L. In some embodiments, iron is provided as a salt. One exemplary salt is FeSO₄. Those of skill in the art will readily know of other salts that can be used, as this aspect of the invention is non-limiting. The salt is preferably pharmaceutically acceptable.

In some embodiments, the medium comprises vanadate at a concentration of about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 nM. In some embodiments, the medium comprises vanadate at a concentration greater than about 0.1, 0.2, 0.3, 0.4, 0.5, 0.6, 0.7, 0.8, 0.9, 1.0, 1.1, or about 1.2 nM. In some embodiments, the medium comprises vanadate at a concentration of about 0.1-5.0, 0.1-2.5, 0.5-2.0, 0.5-1.5, 0.8-1.0, or about 0.9-1.0 nM. In some embodiments, the medium comprises cobalt at a concentration of about 0.98 nM. In some embodiments, vanadate is provided as a salt. One exemplary salt is Na₃VO₄. Those of skill in the art will readily know of other salts that can be used, as this aspect of the invention is non-limiting. The salt may be pharmaceutically acceptable.

### Additional Media Considerations

The medium may be supplied as a liquid including for example as a concentrated liquid intended to be diluted with an aqueous solution (e.g., water). The levels and concentrations provided herein are intended for use in a 1× stock of liquid media. The levels and concentrations necessary to achieve a more concentrated stock will therefore be clear to one of ordinary skill based on these teachings. Such concentrated stocks may be 5×, 10×, 20×, 50× or 100× stocks without limitation. Typically such stocks would be diluted with an aqueous solution such as water, and may require pH adjustment. When supplied as a liquid, the media is typically provided in a bottle, and such bottle may be plastic or glass, including clear and colorless or colored to protect the media from light. Also when supplied as liquid, the media may be provided and/or stored at 4° C.

Alternatively, the medium may be supplied as a solid such as a powder. In this form, the medium may be provided in a packet, and such packet may protect the medium from light. The medium may also be provided with instructions regarding its reconstitution in an aqueous solution such as water, and optionally any pH adjustment that may be performed. Following reconstitution, the media may be sterilized (e.g., filter sterilized). The media may be provided and/or stored at 4° C.

The concentrations provided herein intend the concentration of media supplied as a liquid. One of ordinary skill in the art will understand that the media may be provided as a solid form and thereby comprise component levels required for reconstitution in a fixed volume such as for example a liter of a liquid carrier. Thus, as an example, if a concentration of 50 mM of ammonium is indicated and if the media is supplied as a solid form, then 50 millimoles of ammonium are supplied. The concentrations are also intended to be in an amount to yield that concentration when diluted if the media is supplied as a concentrated liquid. For example, if the concentration provided is 50 mM ammonium and the media is supplied as a 5× concentrated liquid, the concentrated liquid will have 250 mM ammonium.

In some embodiments, the media has a pH of about 3-8. In some embodiments, the media has a pH of about 5-7.5. In some embodiments, the media has a pH of about 6.5. The pH may be the pH of the media without any adjustment or it may be the pH attained after adjustment.

One or more components of the media described herein may be provided separately from the remainder of the media components. For example, arginine and/or glutamine may be supplied separately from the remainder of the media components. As another example, the one or more lipids may be supplied separately from the remainder of the media components. As yet another example, the one or more vitamins may be supplied separately from the remainder of the media components.

### Methods of Use

In some embodiments, the medium described herein is useful for culturing one or more cells, typically a plurality or population of cells. In some embodiments, the method comprises introducing one or more cells including a cell population described herein into the medium described herein. In some embodiments, the media described herein can be used to cultivate one or more cells.

In some embodiments, the cell is a prokaryotic cell. Non-limiting examples of prokaryotic cells include cyanobacteria algae and bacteria. The bacterium may be a gram-negative bacterium, including, but not limited to, including Escherichia, Salmonella, Shigella, Pseudomonas, Neisseria, Chlamydia, Yersinia, Moraxella, Haemophilus, Helicobacter, Acinetobacter, Stenotrophomonas, Bdellovibrio, Legionella, and acetic acid bacteria. In other embodiments, the bacterium may be a gram-positive bacterium, including, but not limited to, Streptococcus, Staphylococcus, Corynebacterium, Listeria, Bacillus, Clostridium, Lactobacillus, and Mycobacterium.

In some embodiments, the cell is a lower eukaryote. Lower eukaryotes include yeast, fungi, collar-flagellates, microsporidia, alveolates (e.g., dinoflagellates), stramenopiles (e.g., brown algae, protozoa), rhodophyta (e.g., red algae), plants (e.g., green algae, plant cells, moss) and other protists.

In some embodiments, the cell is a filamentous fungi. Non-limiting examples of filamentous fungi include Trichoderma, for example from Trichoderma reesei; of Neurospora, for example from Neurospora crassa; of Sordaria, for example from Sordaria macrospora; of Aspergillus, for example from Aspergillus niger, Aspergillus nidulans, Aspergillus oryzae, or from Aspergillus sojae; of Fonsecaea, for example from Fonsecaea pedrosoi; of Cladosporium, for example Cladosporium carrionii; Chrysosporium luchiowense; Fusarium sp. (for example, Fusarium gramineum, Fusarium venenatum); Physcomitrella patens; or from Phialophora, for example Phialophora verrucosa.

In some embodiments, the cell is a yeast cell. Examples of yeast include, but are not limited to, Arxula adeninivorans, Aureobasidium pullulans, Aureobasidium melanogenum, Aureobasidium namibiae, Aureobasidium subglaciale, Brettanomyces bruxellensis, Brettanomyces claussenii, Candida albicans, Candida auris, Candida bracarensis, Candida bromeliacearum, Candida dubliniensis, Candida glabrata, Candida humilis, Candida keroseneae, Candida krusei, Candida lusitaniae, Candida oleophila, Candida parapsilosis, Candida rhizophoriensis, Candida sharkiensis, Candida stellate, Candida theae, Candida tolerans, Candida tropicalis, Candida ubatubensis, Candida viswanathii, Candida zemplinina, Cryptococcus gattii, Cryptococcus neoformans, Debaryomyces hansenii, Hansenula polymorpha, Hanseniaspora guilliermondii, Kluyveromyces lactis and like kinds, Kluyveromyces marxianus, Leucosporidium frigidum, Macrorhabdus ornithogaster, Malassezia caprae, Malassezia dermatis, Malassezia equine, Malassezia japonica, Malassezia nana, Malassezia sympodialis, Ogataea methanolica, Ogataea polymorpha, Pachysolen tannophilus, Pichia anomala, Pichia guilliermondii, Pichia pastoris, Pichia stipites, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Rhodotorula cladiensis, Rhodotorula evergladiensis, Saccharomyces bayanus, Saccharomyces boulardii, Saccharomyces cerevisiae, Saccharomyces paradoxus, Schizosaccharomyces pombe, Yarrowia lipolytica, and Zygosaccharomyces bailii. In one embodiment, the yeast is Pichia pastoris.

Various yeasts, such as K. lactis, Pichia pastoris, Pichia methanolica, and Hansenula polymorpha, are currently preferred for cell culture because they are able to grow to high cell densities and secrete large quantities of recombinant protein. Likewise, filamentous fungi, such as Aspergillus niger, Fusarium sp, Neurospora crass, and others can be used to produce recombinant proteins at an industrial scale.

Pichia pastoris, a species of methylotrophic yeast, is used predominantly for protein production with recombinant DNA techniques. It possesses a small, tractable 9.4 MB genome that can be easily manipulated with an established toolbox of genetic techniques. Unlike other eukaryotic expression systems, it can produce disulfide bonds and glycosylation in proteins and thus is well-suited for the large-scale production of recombinant eukaryotic proteins. Two useful P. pastoris strains include Komagataella pastoris and Komagataella phaffii. It is to be understood that the recitation of Pichia generally herein intends to embrace K. pastoris and K. phaffii, and that any teachings relating to Pichia apply equally to either of these strains. It should also be understood that typically only one of these strains will be used as a host cell rather than a mixture of these strains.

As a methylotroph, P. pastoris can be grown with methanol as its only source of energy, a solute that would kill most other microorganisms. The genes involved in methanol utilization are repressed by the presence of glucose, and derepressed but not activated in glycerol. It can also be grown to ultra-high cell densities; under ideal conditions, it can multiply to the point where its cell solution is essentially paste. The yeast also does not have endotoxins, as found in bacteria, or viral contamination risks, as seen in proteins produced in animal cell culture. It further only requires media containing one carbon source and one nitrogen source, making it suitable for isotopic labelling applications, including protein NMR. Therefore, it is a preferable organism for the generation of eukaryotic proteins.

The specific growth rate of P. pastoris cultures has been shown to play a role in gene regulation and protein production. High growth rates have been shown to be beneficial for protein production in the yeast, which leads to the upregulation of genes relating to gene expression and translation and the downregulation of genes associated with catabolic processes (autophagy, transport to peroxisome, and mitochondrial degradation, for example).

In some embodiments, Pichia has one or more promoters that are regulated by carbon source. Such promoters include OLE1, DAS2, AOX1, and GAPDH promoters. For example, the AOX1 promoter is regulated by methanol. In such embodiments, the Pichia will produce a protein under the control of the AOX1 promoter when the cells are exposed to media containing methanol. Methanol inducible fermentation systems based on the AOX1 promoter are based on the use of glycerol as a substrate for biomass growth, followed by a methanol feed for induction. In some embodiments, a multistage fermentation process including a glycerol growth phase, transition phase, and methanol production phase is employed for the production of recombinant proteins using a Pichia production strain. In other embodiments, a glycerol growth phase is followed by a methanol production phase with no transition phase. In any given expression vector system, the titer (i.e. amount of desired protein product produced) is largely dependent upon cell growth and feeding methods of primary carbon sources including the use of methanol as an inducer.

In some embodiments, cells are grown on fermentable carbon sources. Fermentable carbon sources induce growth of Pichia. Fermentable carbon sources can also be used to induce expression of proteins under the regulation of promoters controlled by the fermentable carbon sources. Suitable carbon-energy sources for growing Pichia pastoris include but are not limited to the carbon-energy source selected from the group consisting of methanol, glycerol, sorbitol, glucose, galactose, raffinose, sucrose, trehalose, lactic acid, ethanol, oleic acid, xylose, xylitol, inulin, gluconate, and fructose and combinations of any two or more thereof. Preferred carbon-energy sources for growing Pichia pastoris are carbon-energy sources selected from the group consisting of methanol, glycerol, and combinations thereof.

In some embodiments, a non-fermentable carbon source is added to 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5%, 6%, 7%, 8%, 9%, 10% to the media described herein. In some embodiments, the media contains 0-4% glycerol. In some embodiments, the media contains 0.1%, 0.5%, 1.0%, 1.5%, 2%, 3%, or 4% glycerol. In some embodiments, the media contains 0-3% methanol. In some embodiments, the media contains 0.1%, 0.5%, 1.0%, 1.5%, 2.0%, 2.5%, or 3.0% methanol.

In some embodiments, cells are grown on a non-fermentable carbon source. Although a non-fermentable carbon source will not induce growth of Pichia or will have limited effect of growth of Pichia, a non-fermentable carbon source can be used to induce expression of heterologous proteins under the regulation of promoters controlled by the non-fermentable carbon sources. Examples of non-fermentable carbon sources include sugars and sugar alcohols such as, for example, arabinose, D-maltose (maltobiose), maltitol, D-gluconic acid, D-sorbose, D-ribose, myo-inositol, L-mellibiose, and quinic acid. This disclosure contemplates use of the media provided herein with a first carbon source during the growth or exponential phase of culture and a second carbon source during the production phase of culture. The first carbon source may be a fermentable carbon source such as glycerol, and the second carbon source may be a non-fermentable carbon source such as but not limited to those recited above.

In some embodiments, cells are grown on more than one carbon source, sequentially or simultaneously. This can be beneficial, for example, during protein production, where it is desirable to have a growth phase during which the growth of the cells is maximized and a production phase during which the production of the protein is maximized. For example, when Pichia pastoris is being used to produce a protein of interest, in some embodiments, the protein of interest will be placed under the control of the AOX1 promoter, which is regulated by methanol. Thus, in some embodiments, the Pichia yeast will first be grown in a media other than methanol, e.g., glycerol, during the growth phase and then will be introduced to media containing methanol to induce production of the protein under the control of the AOX1 promoter.

In some embodiments, the cells are grown in a medium containing a single carbon source. In some embodiments, the cells are grown in a medium containing more than one carbon source. In some embodiments, cells will first be grown in a media containing a first carbon source and will then be grown in media containing a second carbon source. In some embodiments, the cells are first grown in media containing a first carbon source, are then grown in media containing a mixture of a first and second carbon source. In some embodiments, the cells are first grown in a media containing a mixture of a first and second carbon source and are then grown in media containing a second carbon source. In some embodiments, cells are first grown in media containing a first carbon source, are then grown in media containing a mixture of a first and second carbon source, and are then grown in a media containing a first and second carbon source.

In some embodiments, the cells contain a gene expressing a protein of interest under the methanol-induced promoter and the cells are first grown in media containing glycerol or another fermentable carbon source other than methanol during a growth phase and are then grown in media containing methanol during a production phase. The cells may, for example, be grown to a desired cell density (e.g., OD₆₀₀ 0.2-1.0) in about 1% to about 10% (e.g. about 1%, 2%, 3%, 4%, 5%, 6%, 7%, 8%, 9%, or 10%) glycerol, the medium is switched to a medium containing a different carbon source (e.g., methanol), which activates expression of genes under control of an inducible promoter, such as OLE1, DAS2, and AOX1. The methanol concentration may vary from about 0.01% to about 10% (e.g. 0.01%-0.1%, e.g. 0.01%, 0.02%, 0.03%, 0.04%, 0.05%, 0.06%, 0.07%, 0.08%, 0.09%, 0.1%, e.g., 0.1%-1%, e.g., 0.2%, 0.3%, 0.4%, 0.5%, 0.6%, 0.7%, 0.8%, 0.9%, 1.0%, e.g., 1%-10%, e.g. 2%, 3%, 4%, 5%, 6%, 7%, 8%, 9%, 10%).

Cells described herein can be grown in the media described herein in various containers and culture systems.

In some embodiments, the cells described herein are grown in the media described herein in plates. In some embodiments, the cells are grown in 24-well plates.

In some embodiments, the cells described herein are grown in the media described herein in a shake flask. A shake flask is a flask with a round or beveled bottom which is being shaken at a controlled rate to aerate the culture and to prevent sedimentation of the cells.

In some embodiments, the cells described herein are grown in the media described herein in a bioreactor. In some embodiments, bioreactors comprise systems for cultivating microbial cultures and/or obtaining and/or processing biologic materials. In some embodiments, a bioreactor is a system for obtaining a protein of interest from a cell culture producing the protein of interest. For example, in the case of Pichia pastoris expressing a protein of interest under the control of the AOX1 promoter, the Pichia yeast can be introduced into a bioreactor in growth phase on glycerol media and transitioned to methanol media to initiate protein production. Media containing the protein of interest can then be collected from the bioreactor.

In some embodiments, a bioreactor comprises a batch bioreactor. In some embodiments, a bioreactor comprises a fed-batch bioreactor. In some embodiments, a fed-batch bioreactor comprises a cell culture into which nutrients, e.g., carbon sources, are added. In a fed-batch bioreactor, the protein of interest can be collected by collecting the cell culture at the end of the production run.

In some embodiments, a bioreactor comprises a continuous flow bioreactor. In some embodiments, a continuous flow bioreactor comprises a cell culture into which fresh media is flowed and out of which cell culture is collected at approximately equal rates. Accordingly, changes in carbon source can be accomplished by changing the carbon source in the media being flowed into the system. Cell culture media containing the protein of interest can be continuously collected from the cell culture.

In some embodiments, a continuous flow bioreactor comprises a chemostat. In some embodiments, the continuous flow bioreactor comprises a perfusion bioreactor.

In some embodiments, cells grown as described herein will have a dissolved oxygen level of 1-100%. In some embodiments, cells grown as described herein will have a dissolved oxygen level of about 80-100%, 70-90%, 60-80%, 50-70%, 40-60%, 30-50%, 20-40% or about 10-30%. In some embodiments, cells grown as described herein will have a dissolved oxygen level of about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%, 45%, 50%, 55%, 60%, 65%, 70%, 75%, 80%, 85%, 90%, 95%, or 100%.

In some embodiments, the methods involve perfusing media of increasing concentration for one or more of the foregoing culture components including ammonium, lipids, glutamine, arginine and vitamins. As discussed herein, the amounts of at least some of these components should be kept low during initial stages of culture having less biomass and can be increased as biomass increases. Thus the methods contemplate increasing the concentration of one or more of the foregoing components during the culture period based in part on the cell density in the culture (or biomass). Such increased additions may be made as step gradients or as continuous gradients. It is to be understood that the concentrations of culture components would still be within the ranges set forth herein even for maximum cell density cultures.

### Methods of Production

Provided herein are methods of producing the media described herein.

In some embodiments, producing the media comprises reconstituting the media. In some embodiments, the media is provided as a powder, a solid, or as a concentrated liquid, and the media is reconstituted in a liquid carrier.

In some embodiments, one or more components of the media described herein are provided separately from the remainder of the media components and producing the media comprises adding to the media the one or more components provided separately from the remainder of the media components. For example, lipids cannot be supplied as a powder. As such, when the media is supplied as a powder, the lipids must be supplied separately. In some embodiments, the one or more lipids are supplied separately from the remainder of the media components and are added to the media. In some embodiments, the one or more vitamins are supplied separately from the remainder of the media components and are added to the media. In some embodiments, arginine or glutamine are supplied separately and are added to the remainder of the media components and are added to the media.

In some embodiments, producing the media comprises sterilizing the media. In some embodiments, sterilizing the media comprises filter sterilizing the media. In some embodiments, sterilizing the media comprises autoclaving the media. In some embodiments, one or more media components provided separately from the remainder of the media components cannot be autoclaved and are not added to the media until after autoclaving. In some embodiments, the one or more vitamins are added to the media after autoclaving. In some embodiments, the one or more lipids are added to the media after autoclaving. In some embodiments, the arginine or glutamine are added to the media after autoclaving.

In some embodiments, producing the media comprises adjusting the pH of the media. The pH of the media can be adjusted using any suitable acid or base. Techniques for adjusting the pH of media are well known to those of skill in the art. In some embodiments, the pH of the media will be adjusted to a pH of about 3-8. In some embodiments, the pH of the media will be adjusted to a pH of about 5-7.5. In some embodiments, the pH of the media will be adjusted to a pH of about 6.5.

### Kits

Provided herein are kits comprising the media.

In some embodiments, the media is in liquid form. In some embodiments, the media is in powder or solid form. In some embodiments, the media is in concentrated liquid form.

In some embodiments, the media is in liquid form and the media has been filter sterilized. In some embodiments, the media is in liquid form and the media has been sterilized by gamma irradiation. In some embodiments, the media is in concentrated liquid form and the media has been filter sterilized. In some embodiments, the media is in concentrated liquid form and the media has been sterilized by gamma irradiation. In some embodiments, the media is in liquid form and the media has been autoclaved. In some embodiments, the media is in concentrated liquid form and the media has been autoclaved. In some embodiments, the media is in powder or solid form and the media has been autoclaved. In some embodiments, the media is in powder or solid form and the media has been sterilized by gamma irradiation. In some embodiments, the media is in powder or solid form and the media has been sterilized using ethanol.

In some embodiments, one or more components of the media described herein are provided separately from the remainder of the media components.

In some embodiments, the one or more lipids are supplied separately from the remainder of the media components. In some embodiments, the one or more lipids are provided in a liquid form. In some embodiments, the one or more lipids have been filter sterilized.

In some embodiments, the one or more vitamins are supplied separately from the remainder of the media components. In some embodiments, the one or more vitamins are supplied in a liquid form. In some embodiments, the one or more vitamins have been filter sterilized. In some embodiments, the vitamins are supplied in a powder or solid form.

In some embodiments, arginine or glutamine are supplied separately from the remainder of the media components. In some embodiments, arginine or glutamine are supplied in a liquid form. In some embodiments, arginine or glutamine have been filter sterilized. In some embodiments, arginine or glutamine are supplied as a powder or solid form.

In some embodiments, the kit further comprises cells. In some embodiments, the media further comprises yeast or filamentous fungal cells described herein. In some embodiments, the media further comprises Pichia pastoris cells described herein. In some embodiments, the cells are provided as a stab in solid media, in a petri dish containing solid media, in frozen stocks, lyophilized, freeze dried, or in liquid media.

In some embodiments, the kit further comprises one or more of the carbon sources described herein. In some embodiments, the carbon source is pre-mixed with the media. In some embodiments, the carbon source is provided separately from the media. In some embodiments, the carbon source is provided separately from the media in a liquid form. In some embodiments, the carbon source is provided separately from the media in a powder or solid form.

In some embodiments, the media is provided in a bottle. In some embodiments, the media is provided in containers such as vials, tubes, bottles, e.g., polypropylene or glass bottles, bags, pouches, boxes, cartons, drums. In some embodiments, the containers are light protected.

In some embodiments, the one or more components of the media provided separately from the remainder of the media components are provided in containers such as vials, tubes, bottles, bags, pouches, boxes, cartons, drums. In some embodiments, the containers are light protected.

In some embodiments, the kits further provide instructions for using the kit. In some embodiments, the kit provides instructions for reconstituting the media. In some embodiments, the kit provides instructions for cultivating the cells.

### Transcriptomic-Based Methods Including Media Development

This disclosure further provides transcriptomics-based methods for analyzing a cell or a cell population and/or its environment and/or its response to environmental conditions. As a specific example and as described herein, a transcriptomics-based method was used to identify genes that are upregulated (or downregulated) in one environment (such as a culture condition) relative to another environment. As another example and as described herein, a transcriptomics-based method was used to identify genes that are upregulated (or downregulated) at one timepoint during fermentation relative to another timepoint. The identification of such genes (or clusters of genes) can be used to identify, for example, components which are so lacking in an environment (such as a culture condition) that the cell population must synthesize such components endogenously. One approach to address this issue is to engineer the cell(s) genome to compensate for this environmental deficiency (e.g., to overexpress one or more enzymes necessary to produce a metabolite). Similarly, such genes (or clusters of genes) can be used to identify, for example, components which are so lacking at a particular time during fermentation that the cells must synthesize such components endogenously. Another approach is to modify the environment of the cell(s). This latter approach is demonstrated in the Examples and involves analyzing a cell population, identifying an expression profile that indicates a deficiency in the cell environment, and then supplementing that environment to compensate for the deficiency. By doing so, the cell population may be more robustly cultured resulting in a larger biomass in shorter periods of time. Additionally, if the cell population is used as host cells for the production of heterologous proteins, then supplementing a culture with the components the cell would be synthesizing endogenously may reduce background protein load that can interfere with harvest and purification of the heterologous protein. More importantly, the transcriptional machinery and substrates in the cell can be redirected to the production of the heterologous protein of interest, thereby resulting in increased production of such protein.

Thus, it will be understood that a cell or cell population may be analyzed at the level of the transcriptome in order to determine the cell status in a particular environment. This analysis can also be used to identify stressors experienced by the cell under a particular environment for the purpose of alleviating such stressors by modifying the environment. The metabolic requirements of a cell population may change during different phases of growth, such as for example the growth or exponential phase and the lag or production phase in the context of host cells engineered to produce a heterologous protein. Protein production may be improved by determining the cellular requirements at the different phases, and where possible exogenously providing such requirements.

This disclosure further illustrates the use of a transcriptomics-based approach to compare culture conditions and thereby identify differences between culture conditions that might not otherwise be readily apparent. As a specific example and as described herein, a cell population was cultured in parallel in a minimal chemically defined culture media and in a complex (i.e., undefined) media. In some instances, complex media can yield a more robust growth of cells as indicated by increased growth (doubling) rate and/or increased biomass (total mass of cells in a culture, for example) as compared to minimal media. The specific differences between these media are not always readily apparent and thus it is difficult, if not impossible, to supplement the minimal medium with defined components from the complex media. However, by comparing the transcriptome of cells cultured in a minimal medium with the transcriptome of cells cultured in a complex medium, the inventors have shown that such transcriptomes are different and more importantly that they reveal components that may be present in the complex media but lacking or limiting in the minimal media. The minimal media may then be supplemented with such identified components in order to arrive at a more robust chemically defined media. Production of heterologous proteins can be improved as a result. In some instances, the carbon sources used to grow the cell populations are the same.

As another specific example and as described herein, gene expression was characterized in a cell population at different stages of perfusion. By comparing the transcriptome of cells at different times during perfusion cultivation, the inventors have shown that such transcriptomes are different and more importantly that they reveal that after prolonged growth on methanol, certain biological processes are downregulated. Based on these results, the methanol level was increased and heterologous protein production increased as a result. This is yet another example of how the transcriptome can be used to define cellular requirements and/or inform an end user of cellular experiences during cultivation.

The approach provided herein that relies on the cell transcriptome to reveal the cellular requirements and drive the modification of the environment (e.g., cell culture) can be contrasted with a more empirical approach for optimizing culture conditions. The empirical approach may involve simply incubating cells with particular culture components and determining the effect of the components on the cells. This approach can be more labor-intensive, time-intensive and more expensive, and it may not ultimately yield a complete profile of the cell requirements.

Based on the foregoing, it should also be clear that the methods provided herein may be used to identify a subset of culture components or a complete culture repertoire for a particular cell population. This approach may include formulating a culture media that comprises growth promoting components and optionally other components that can limit or repress non-productive pathways or pathways that negatively impact heterologous protein production. As an example of the latter instance, a transcriptome analysis may reveal that a proteolysis pathway is active in the cell, and this could be addressed by the inclusion of proteolysis inhibitors in the culture media.

These methods may also be used to determine the cellular requirements of single cells and clonal populations. By analyzing single cells or clonal populations, it may be possible to identify cells having preferred metabolic pathway profiles, including those that are more suited to heterologous protein production (e.g., identifying cells having lower background secretory protein production, or identifying cells that process culture media more efficiently, etc.). Transcriptomic analysis of non-clonal populations can potentially obscure such profiles particularly if a clonal population of interest is under-represented in the population.

As used herein, a transcriptome is a population of transcripts present in a cell or a cell population. Such transcripts are typically RNA, and more specifically mRNA, in nature. Methods for obtaining transcriptomes are known in the art and include microarray-based methods and direct sequencing of transcription products or their complements. An example of the latter technology is RNA-Seq in which poly(A)-enriched strand-specific cDNA is sequenced. In some embodiments, RNA-seq may be performed on single cells, for various purposes including those described above. Sequence clustering may then be performed in order to facilitate comparison between two or more populations, thereby identifying commonalities and/or differences between the populations.

Thus, in some instances, provided herein is a method for improving maintenance and preferably cell growth in vitro. In some instances, provided herein is a method for improving heterologous protein production in vitro. The method may comprise culturing a first cell population under a first condition, obtaining and analyzing expression products from an aliquot of the cultured cell population, identifying expression products having an altered expression level relative to a control, and modifying the first culture condition by addition and/or deletion of one or more culture components based on the identity of the expression products having an altered expression level.

The method may involve identifying differences between a cell population that is cultured in two different culture conditions (i.e., a first and a second culture condition which are different from each other). The difference between the culture conditions may not be known a priori and the method therefore allows an end user to gain insight into such differences. Alternatively, the differences between the culture conditions may be known and the method is intended to determine and/or identify any disparate downstream effects that result from the culture control differences. Thus, the control in this situation is the expression products obtained after culturing the same cell population under a different (e.g., second) condition. The first culture condition may be a minimal chemically defined culture medium and the second culture condition may be a complex (undefined) culture medium. Examples of such media are known in the art, although the methods are not limited to use of such existing media and instead may be applied to virtually any media formulation or culture condition.

The method may also involve identifying differences between different stages of growth of a cell population. For example, differences may be identified between lag phase and exponential growth phase on the same carbon source, or between a growth phase and a phase of protein expression using a carbon source or other additive that promote each of these distinct phases. The method may be intended to determine and/or identify disparate downstream effects that result from the growth stage and/or amount and/or type of carbon source or additive. The cell population may be virtually any cell population that can be grown in vitro. The Examples provided herein focus on Pichia pastoris strain analysis but the method may be carried out using any prokaryotic or eukaryotic cell type.

In other instances, the method may involve identifying differences between two different cell populations (i.e., a first and a second cell population which are different from each other) cultured under the same culture condition. The difference(s) between the two cell populations may or may not be known. For example, the two populations may be different microorganisms, different strains of a microorganism, and different mutational variants of a given strain. A specific example is the two strains K. pastoris and K. phaffii that are typically grouped under the Pichia pastoris name. The culture condition may be a minimal chemically defined culture medium, or it may be a complex (undefined) culture medium.

The expression products, regardless of composition, may be involved in cellular metabolism including for example central carbon metabolism. For example, the expression products may be involved in fatty acid synthesis, vitamin synthesis, amino acid synthesis, nucleotide synthesis, protein glycosylation, redox biochemistry, or electron transport.

The expression products are defined as any products synthesized by the cells or cell population that can be detected and measured. In some instances, the expression products comprise RNA such as mRNA. In other instances, the expression products comprise proteins such as but not limited to proteins required for cellular metabolism (e.g., certain enzymes in biosynthetic pathways). In still other instances, the expression products comprise metabolites, carbohydrates, and the like.

The expression products having an altered expression level may be identified as reporter metabolites as defined by a genome-scale metabolic model (GEM). Other approaches that may be used for gene expression data analysis including transcript clustering include GO-slim and custom gene grouping. Reference can be made for example to Love et al. BMC Genomics 2016, 17:550; Tomas-Gamisans and Albiol, PLoS One, 2016 Jan. 26, 11(1); and Liang et al. BMC Genomics 2012, 13:738 for various approaches for collection and analysis of transcriptomic data. The methodologies relating to transcriptomic analysis, gene expression analysis, and gene clustering provided therein are incorporated by reference herein in their entirety.

The expression products having an altered expression level may be those that are upregulated or downregulated depending on the particular analysis and application. In the analysis described herein, expression products having an increased expression level (in the presence of a minimal medium as compared to a complex medium) are of interest as they are indicative of an active biosynthetic pathway in the cell population. More specifically, in this analysis, expression products having an altered expression level were defined as expression products having a log 2 fold change that is greater than 2 with an adjusted p-value of less than 0.05. Other cutoffs may be used in other applications, as will be apparent to those of skill in the art.

The methods may comprise inclusion or exclusion of one or more culture components. The methods may alternatively comprise increasing or decreasing the level (e.g., concentration) of one or more culture components. It will be understood that the methods therefore contemplates increasing the level of a component that is already included in a culture condition or decreasing the level of a component to a non-zero level. Such changes will be based on the identity of the expression products having an altered expression level. It should be apparent that the expression products are typically not the actual culture components but rather the cellular machinery, including enzymes, that are required in order to synthesize the endogenous counterparts of such components.

Example 1 provides one such transcriptomics-based analysis. As documented herein, two distinct groups of reporter metabolites were detected based on differential expression levels. One group included the reporter metabolites butanoyl-CoA, octadecynoyl-CoA, tetradecenoyl-CoA, hexadecenoyl-CoA, octadecenoyl-CoA and acetyl-CoA. This reporter grouping indicated that Pichia cells cultured in a chemically defined medium (referred to herein as Generation 1 medium) were making lipids endogenously, as compared to Pichia cells cultured in a complex medium. Thus, the Generation 1 media was then further supplemented with one or more lipids in order to curtail the endogenous lipid and fatty acid biosynthesis in this cell population. A second reporter grouping indicated that Pichia cells cultured in the Generation 1 medium were making certain vitamins endogenously, as compared to Pichia cells cultured in a complex medium. The reporter metabolites so identified are provided in Table 5. Gene groupings that correlate with particular metabolites are known in the art. Reference can be made for example to Tomas-Gamisans and Albiol, PLoS One, 2016 Jan. 26, 11(1) as well as other gene ontogeny methodology (see for example the Gene Ontogeny (GO) Slim and subset guide at the Gene Ontogeny Consortium website.

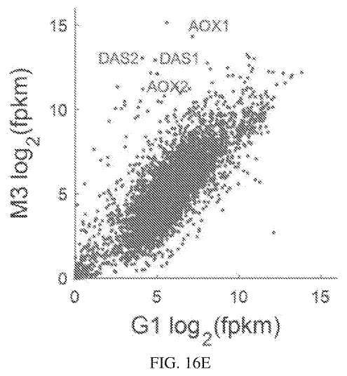

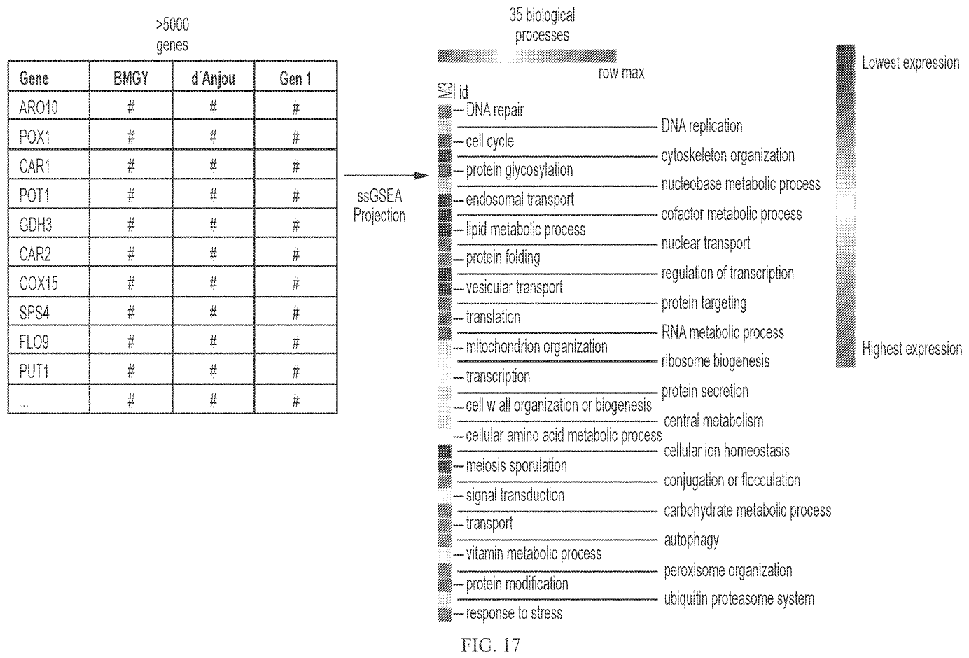

Example 3 provides another such transcriptomics-based analysis. As documented herein, changes in biological processes, which clustered into co-expressed groups were seen when gene expression was characterized at different stages of perfusion cultivation. Gene expression was characterized in a cell population at different stages of perfusion cultivation including during growth in glycerol and after induction in methanol. Samples were taken from all reactors after one day of exponential growth on glycerol (G1) and immediately after the DO spike (G2). For a subset of bioreactors, glycerol feed was maintained for 4 hours after the DO spike, and samples were taken at the end of this period (G3). For the other bioreactors, samples were taken after 4 hours of methanol feeding (M1). Samples were taken from all bioreactors one day (M2) and two days (M3) after the start of methanol feeding. The grouping of changes in biological processes are shown in FIG. 17 and indicated that in the exponential growth state (G1), the cells displayed the highest expression of genes involved in DNA replication and repair, cell cycle, and central metabolism. At G2 and G3, growth-associated processes were down-regulated and expression increased in processes that reflect reduced nutrient availability including autophagy, transport, carbohydrate metabolism, and amino acid metabolism. At M1, autophagy, transport, and carbohydrate metabolism remained up-regulated, while central metabolism and amino acid metabolism were no longer as highly expressed. There was a significant up-regulation of peroxisome organization and vitamin metabolism. After prolonged growth on methanol (M3), heterologous protein production was down-regulated, vitamin metabolism was down-regulated and response to stress was up-regulated. This indicates that cells are experiencing carbon stress at M3. Based on these results, the methanol level was increased and heterologous protein production increased as a result.

Based on the expression products having an altered expression level, the end user may choose to modify one or more culture components such as one or more lipids, one or more vitamins, one or more amino acids, one or more nucleotides or nucleosides, and the like. Additionally or alternatively, the end user may choose to modify the amount or type of carbon source.

It will be readily apparent to those of ordinary skill based on the disclosure provided herein that these transcriptomic-based methods may be used for a variety of applications including those that seek to optimize cell maintenance and growth as well as those that might seek to control, limit and/or repress the growth of and/or kill a certain cell population. Other applications may be directed at optimizing cell function including endogenous functions (e.g., cell metabolism) or exogenous functions (e.g., production of a heterologous protein of interest). Based on the information obtained from such proteomics-based analysis, the environment of the cell may be altered by the introduction of components other than metabolites or carbon sources. For example, a culture medium may be supplemented with additives that control cellular processes to increase yield and potentially quality of the protein of interest. One example may be the addition of protease inhibitors when a proteolysis pathway appears active or upregulated in a cell population, or adding proteasome inhibitors when endoplasmic reticulum-associated protein degradation (ERAD) or ubiquitination processes appear active or upregulated in the cell population. Thus the methods provided herein can be used to identify additives for media formulation that promote cell growth and heterologous production and/or secretion as well as other agents that may be used to suppress non-productive pathways occurring in the cell.

### EXAMPLES

### Example 1: Development of a General Defined Media for Pichia pastoris

Pichia pastoris is a popular expression host for recombinant proteins and holds great potential for manufacturing of biologic drugs. Compared to the current standard host, Chinese hamster ovary (CHO), Pichia pastoris grows much more quickly and to higher cell densities. As a eukaryote, Pichia contains the necessary cellular machinery for protein folding, glycosylation, and secretion so it can be used to produce complex proteins used as therapeutics. It has been engineered to produce human-like glycoforms (Gerngross, 2004). As a fast-growing and robust host organism, Pichia can enable faster production cycles, agile process development, and potentially lower production costs. However, productivities are currently lower than those achieved with CHO cells.

Media optimization has not been widely explored for Pichia. Regardless of host organism, defined media is preferred for a number of reasons as described herein. Standard defined media formulations for Pichia fermentation are minimal salt solutions from which the organism synthesizes all metabolic intermediates. The most commonly-used formulation for fermentation of Pichia pastoris is the basal salts medium (BSM) described in the Invitrogen manual (Invitrogen Corporation, 2002). Alternative media formulations such as FM22 (Stratton et al., 1998) and d'Anjou medium (d'Anjou and Daugulis, 2000) have been developed and adopted by some other researchers.

Complex medium is used for preparation of fermenter inoculums and for strain development because the rich components reduce metabolic load and accelerate cell growth. Buffered complex media consists of a carbon source, a buffer, yeast nitrogen base (YNB), peptone, and yeast extract. YNB is defined but peptone and yeast extract have not been fully characterized. Peptone is an enzymatic digest of protein derived from animals or plants. Some peptone manufacturers provide molecular weight distributions and total amino acid content but full characterization is not feasible (Davami et al., 2014). Yeast extract is usually produced through the autolysis of baker's or brewer's yeast and is known to contain peptides, growth factors, and carbohydrates (Zhang et al., 2003). The composition of yeast extract varies significantly across lots, which can affect fermentation performance (Zhang et al., 2003). A better understanding of the composition of complex components may aid in the development of a rich defined media formulation.

Transcriptomic analysis of Pichia has been performed, but its applications to metabolism have been limited so far.

In this work, existing salts formulations were evaluated and characteristics were identified that limited cell growth. Beneficial nutrient supplements were identified and it was shown that a small number of nutrients were sufficient to match the growth rates seen in complex media. Transcriptomics was demonstrated to be an effective tool to characterize nutrient availability and was used to identify additional molecule classes that can further improve growth. The final improved medium demonstrated comparable growth and higher heterologous protein titers as compared to standard complex and minimal formulations.

### Materials and Methods

Batch cultivations were conducted at two scales: microtiter plates and shake flasks. Microtiter plates with 24 wells and pyramidal bottoms were purchased from Axygen. Each well had 10 mL total volume and cultivations used a 3 mL working volume. Plates were incubated in a Liconic LPX44 shaker at 600 rpm. Temperature was not controlled but remained at room temperature, between 21 and 24° C. One liter shake flasks with 200 mL working volume were used in an Innova 4230 shaking incubator. The shake rate was set to 300 rpm and the temperature was controlled at 25° C.

Growth studies were conducted with the Komagataella phaffii wildtype strain (NRLL Y-11430 or ATCC 76273). Production studies were conducted with a strain modified to express human growth hormone under the control of the AOX1 promoter.

All chemicals used in media formulations were purchased from Sigma. Complex media was purchased from Teknova. Media was formulated as previously published unless otherwise specified in the text (d'Anjou and Daugulis, 2000; Invitrogen Corporation, 2002; Stratton et al., 1998). Glycerol was added at a starting concentration of 1% by volume. Media was autoclaved and filtered through a 0.2 m bottletop filter before use. PTM1 salts were added after filtration to avoid loss of trace elements through precipitation.

Biomass was measured by optical density. Shake flask samples were measured on a Genesys 10 Bio spectrophotometer. Microtiter plate samples were measured using a Tecan Infinite M200 Pro plate reader and converted to the same scale as the spectrophotometer samples using a calibration curve. Growth rates were calculated by linear regression between the natural logarithm of the optical density and the cultivation time in hours. The regression was run on time points during the exponential growth phase unless otherwise specified.

Glycerol and ammonium concentrations in the culture supernatants were measured with enzymatic assays (YSI 2950 Bioanalyzer). Free amino acid concentrations in supernatants were measured using o-phthalaldehyde (OPA) and 9-fluorenyl methyl chloroformate (FMOC-Cl) pre-column derivatization high performance liquid chromatography (HPLC) method on an Agilent system. Human growth hormone concentrations in the supernatant were evaluated using gel electrophoresis on a Caliper GXII instrument. Standard reference material was purchased from Sandoz.

RNA was extracted using a Qiagen RNeasy kit and genomic DNA clean-up was performed using DNAse and RNA beads. RNA sequencing libraries were constructed using Neoprep and sequenced on the Illumina NextSeq platform to generate 75-nucleotide paired-end reads at a read depth of at least 3 million reads per sample. Each raw data set was down-sampled to one million paired-end reads. The reads were aligned to the previously reported genome sequence for this strain (Love et al., 2016) using BWA, Bedtools, RSEM, and bowtie2. Unsupervised hierarchical clustering (Ward's Method) was used to examine data relationships for the three biological replicates performed for each cultivation condition. Consistent clustering of biological replicates was observed. Count data for differential expression testing was done using DESeq2 and R. The reporter metabolites algorithm was run with RAVEN toolbox v 1.08 (Cvijovic et al., 2010) using the genome-scale metabolic model for Pichia pastoris published in 2016 (Tomas-Gamisans et al., 2016).

### Results and Discussion

### Evaluation of Salts Formulations

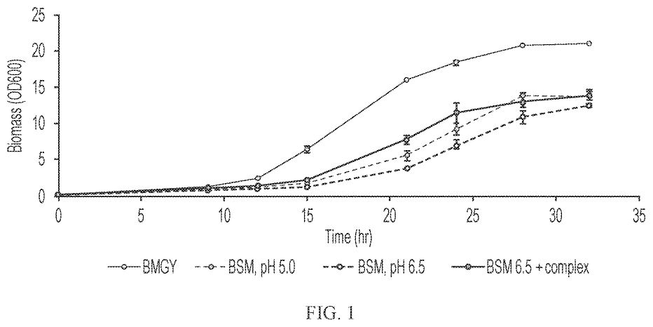

Growth of wildtype Pichia pastoris was slower in basal salts medium than in complex media, with growth rates of μ=0.18 h⁻¹ and μ=Yh⁻¹ respectively (FIG. 1). This difference was not explained by pH, as increasing the basal salts pH from 5.0 to 6.5 slowed growth further to μ=0.094 h⁻¹. The slower growth could not be solely attributed to nutrient limitation, as adding complex components to the higher pH medium still only resulted in a growth rate of μ=0.11 h⁻¹. These results suggest that some characteristic of basal salts inhibits growth.

Since basal salts medium was designed to support high cell densities, concentrations of all major elements were higher than needed for the initial biomass accumulation. Some of these components can be added later in the cultivation without significant volume effects, even in fed-batch format, because they are highly soluble. For continuous cultivations, it is even simpler to feed these elements as they are needed.

The major elements provided by the basal salts medium are nitrogen, sulfur, and phosphorus. The maximum cell density reached in BMGY in the microtiter plate after 24 hours of growth is an OD of 15, which corresponded to a dry cell weight of 8.12 g/L. Pichia typically grows to a density of 350-450 g/L wet cells during fed batch fermentation, so nutrients may be provided at over 50× their required levels for the first 24 hours. For each of the major elements, their concentrations were reduced by 50% and the impact on growth was assessed. Since the nitrogen and phosphorus sources affect the pH, potassium hydroxide and hydrochloric acid were used to vary the elements independently.

As shown in FIG. 2A, reducing ammonium significantly improved the growth rate. Changes to phosphate and sulfur had no significant effect. Further testing showed that the ammonium concentration was negatively correlated with growth until it became limiting, at 12.5 mM (FIG. 2B). While a previous study stated that ammonium only affected the lag phase (Yang et al., 2004), these growth curves show that the growth rate during the exponential phase was also affected.

Even with ammonium concentration lowered to 25 mM, growth in basal salts medium was still significantly slower than in BMGY. Adjusting the pH up to 6.5 with KOH was no longer detrimental, and in fact showed a small benefit. FM22 and d'Anjou media formulations were thus added for consideration. Both have lower salt content than basal salts and the addition of nitrogen and phosphorus is decoupled from the pH, which enables more precise tuning of the nutrient composition.

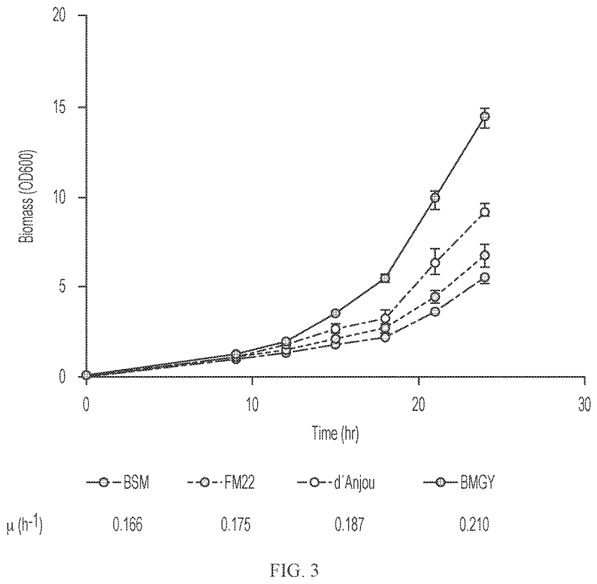

For consistency, the ammonium concentration of all three concentrations was set to 25 mM and the pH was adjusted to 6.5 using KOH. Within these parameters, the growth rate in d'Anjou medium was higher than that of the other established formulations (FIG. 3). There was also less precipitation at pH 6.5, with a blank media OD of 0.24 compared to 1.33 for basal salts and 1.58 for FM22. D'Anjou medium had the lowest salt content of the three formulations, suggesting that osmolarity may hinder biomass accumulation.

### Screening of Nutrient Supplements

Complex medium was used as a starting point to identify nutrient supplements, which were then screened for their impact on growth rate. HPLC analysis of amino acids showed that arginine, alanine, and lysine were the amino acids at highest concentration in complex medium, each of which was present at approximately 5 mM (Table 3). A vitamin solution that has been reported for yeast cultivation was used (Verduyn et al., 1992). Nucleoside concentrations were selected based on previously reported solutions used for cultivation of Tetrahymena thermophila (Hellenbroich et al., 1999). Alternative carbon sources in yeast extract were previously characterized: lactate and trehalose were found to be present at concentrations up to 10 mM and 5 mM respectively in complex medium (Zhang et al., 2003).

TABLE 3
Concentration of amino acids in BGM media

| Amino acid | Concentration (mM) |
|---|---|
| Arginine | 4.2 |
| Alanine | 4.0 |
| Lysine | 3.2 |
| Glycine | 2.7 |
| Glutamate | 2.4 |
| Leucine | 2.3 |
| Phenylalanine | 1.7 |
| Isoleucine | 1.1 |
| Serine | 0.8 |
| Tyrosine | 0.3 |
| Total | 22.8 |

The results of the nutrient screening are shown in Table 4. Glutamine provided the greatest benefit, increasing growth rate from μ=0.196 h⁻¹ to 0.217 h⁻¹. Arginine also provided a significant benefit, while alanine and lysine did not. This result suggests that the nitrogen content of the amino acids may be the determining factor; arginine and glutamine may be taken up more easily than ammonium, while glycerol may be taken up more easily than amino acids as carbon sources.

TABLE 4
Growth rate in supplemented defined media

| Supplement | μ (h⁻¹) |
|---|---|
| None | 0.196 |
| Glutamine | 0.217 |
| Arginine | 0.202 |
| Alanine | 0.195 |
| Lysine | 0.196 |
| Nucleotides | 0.196 |
| Vitamins | 0.204 |
| Lactate | 0.186 |
| Trehalose | 0.193 |
| Complex media | 0.248 |

The vitamin solution also improved the growth rate. The nucleoside solution had no significant effect, indicating that nucleoside metabolism was not limiting.

### Metabolite Analysis of Supplemented Medium

Based on the screening results, a supplemented d'Anjou medium was formulated to include 25 mM ammonium, 5 mM glutamine, 5 mM arginine, and the vitamins solution (referred to as Generation 1 media). This medium was compared to three existing media formulations: buffered complex medium (BMGY), basal salts medium, and d'Anjou medium.

The supplemented medium performed similarly to complex medium for the first 24 hours of growth. Biomass profiles were similar for supplemented medium and complex medium, but slower for the minimal formulations. (FIG. 4A) Growth plateaus were earlier for supplemented medium than for complex, possibly because cells in complex medium can utilize non-glycerol carbon sources in the complex components. Significant acidification was observed in the d'Anjou medium, which had lower osmolarity than the other formulations. However, acidification was greatly reduced in improved medium compared to the original formulation, suggesting that the nutrient supplements had a buffering effect.

Glycerol consumption rate closely followed growth, and glycerol was fully consumed for both the complex and supplemented medium within 24 hours. In supplemented medium, the majority of the ammonium was consumed within the first 24 hours but then leveled off with growth, suggesting that the nitrogen provided was sufficient. (FIG. 4B) In improved medium, amino acids were fully taken up by cells by first time point, after 15 hours of outgrowth. (FIG. 4C.) In complex medium, amino acids were not fully consumed. Raising these concentrations could potentially accelerate growth further or extend its duration.

### Transcriptome Analysis of Supplemented Medium

Changes in media cause significant changes in Pichia's transcriptome. One hundred and twelve genes are differentially expressed between complex and d'Anjou medium, where a gene is considered to be differentially expressed if the adjusted p-value is less than 0.05 and the log 2 fold change is greater than two. Cells grown in the supplemented defined medium have 67 and 68 genes differentially expressed relative to complex and minimal media respectively.

The number of reporter metabolites was also notable: between complex and d'Anjou medium, 164 of 1461 metabolites have p-values less than 0.05. Between complex and supplemented medium, 150 metabolites meet the same criteria, and 67 metabolites meet that criteria between minimal and supplemented medium. Typically only the top 10 metabolites are considered to be reporter metabolites, but the large number of metabolites with very low p-values suggested that a greater number of metabolites may be relevant.

The reporter metabolites accurately reflected known changes in media formulations, as can be seen in the comparison of the minimal and supplemented formulations (Table 5). The top seven reporter metabolites were all involved in vitamin metabolism, specifically synthesis of thiamine, pyrimidine, and biotin. The addition of the vitamin solution seemed to relieve a metabolic requirement on the cell and allow those resources to be directed elsewhere. Arginine and ornithine also appear in the top ten, reflecting the addition of arginine to the medium and its likely conversion into polyamines. Glutamine was also added to the medium and is the twenty-second reporter metabolite, with a p-value of approximately 10⁴. The close linkage between the media supplements and the reporter metabolites confirmed that the transcriptome can reflect differences in metabolic load caused by nutrient availability.

TABLE 5
Transcriptomics analysis of Pichia in different media

| d′Anjou+ vs. d′Anjou | | | | d′Anjou+ vs. complex | | | |
|---|---|---|---|---|---|---|---|
| | | No. of | | | | No. of | |
| Metabolite | Compartment | genes | P value | Metabolite | Compartment | genes | P value |
| 4-Methyl-5-(2- | Cytosol | 3 | <1E−16 | Butanoyl-CoA | Peroxisome | 2 | <1E−16 |
| phosphoethyl)-thiazole | | | | | | | |
| 2-Methyl-4-amino-5- | Cytosol | 4 | <1E−16 | Octadecynoyl-CoA | Peroxisome | 9 | <1E−16 |
| hydroxymethylpyrimidine | | | | | | | |
| diphosphate | | | | | | | |
| Thiamin monophosphate | Cytosol | 2 | <1E−16 | Tetradecenoyl-CoA | Peroxisome | 9 | <1E−16 |
| Dethiobiotin | Cytosol | 1 | 5.55E−16 | Hexadecenoyl-CoA | Peroxisome | 9 | <1E−16 |
| Sulfur | Cytosol | 1 | 5.55E−16 | Octadecenoyl-CoA | Peroxisome | 9 | <1E−16 |
| 4-Methyl-5-(2-hydroxyethyl)- | Cytosol | 2 | 7.66E−15 | Acetyl-CoA | Peroxisome | 10 | <1E−16 |
| thiazole | | | | | | | |
| Biotin | Cytosol | 2 | 2.88E−11 | CoA | Peroxisome | 14 | <1E−15 |
| Ornithine | Cytosol | 10 | 6.63E−11 | NADP | Peroxisome | 6 | <1E−16 |
| L-Arginine | Cytosol | 7 | 9.23E−09 | NADPH | Peroxisome | 6 | <1E−16 |
| 4-Amino-5-hydroxymethyl-2- | Cytosol | 2 | 4.35E−08 | Oxygen | Peroxisome | 9 | <1E−16 |
| methylpryimidine | | | | | | | |

When the same approach was applied to compare the supplemented and complex media formulations, fatty acid metabolism immediately presented itself as a metabolic burden (Table 5). The top six reporter metabolites were coenzyme A-activated fatty acids and the top thirty-eight were all located in the peroxisome. Of individual genes, two of the six most significant differentially-expressed were POX1 and POT1, which catalyze the first and last steps in fatty acid oxidation. This result suggests that fatty acids in complex media serve as a major source of metabolic energy that is lacking from existing defined media formulations.

### Additional Supplements and Protein Production

The impact of fatty acids and additional amino acids on growth rate were first evaluated in microtiter plate format. A lipid mixture from Sigma Aldrich (L0288-100ML), containing seven fatty acids, cholesterol, and surfactants to aid solubility, was added at a concentration of 10 milliliters per liter. The concentrations of glutamine and arginine were doubled, to 10 mM each.

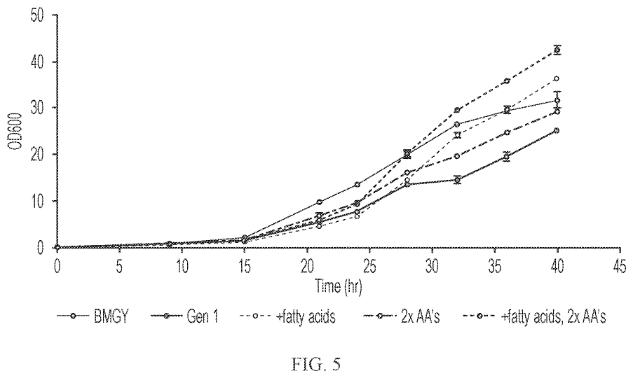

Adding fatty acids showed limited effect during the first 28 hours of growth but then showed a substantial benefit, allowing the continuation of exponential growth at a rate of 0.074 h⁻¹ compared to 0.053 h⁻¹ for the previously described defined media (FIG. 5). In contrast, the addition of amino acids improved the growth rate during the first 24 hours from 0.163 h⁻¹ to 0.178 h⁻¹. The two factors combined resulted in sustained growth and higher cell densities than BMGY, which were not previously achieved in plates.

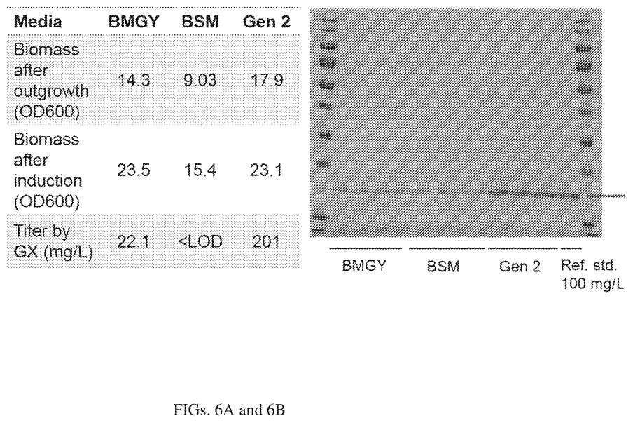

In the shake flask, the growth in improved media is consistent with BMGY, reaching OD of approximately 15 compared to 9 in basal salts medium (FIG. 6A). Growth is sustained beyond 24 hours, which was not the case for the previous defined formulation. Titers of hGH were significantly higher in the improved media than in BMGY and BSM (FIG. 6B). The benefits of the defined media formulation in growth carried over to protein production as well.

### Conclusions

Supplemented media has the potential to increase productivity of Pichia pastoris fermentations both by accelerating growth rates and improving titers, making this host organism more competitive with CHO for therapeutic protein production.

Existing salts formulations for Pichia pastoris are not optimal for biomass accumulation, but can be adapted by reducing ammonium concentrations and osmolarity at the beginning of the cultivation. Analytical methods used to measure consumption rates can inform further optimization of initial concentrations and feed rates.

Transcriptomics is a powerful tool for optimizing media formulations because it accurately reflects metabolic changes caused by different nutrient availability. Fatty acids were the most significant nutrient class highlighted by comparing complex media to defined media, and supplementing that class resulted in sustained biomass accumulation and increased titers of human growth hormone. Transcriptomic analysis is a powerful tool to further optimize the composition of defined production media.

### REFERENCES

Baumann K, Carnicer M, Dragosits M, Graf A B, Stadlmann J, Jouhten P, Maaheimo H, Gasser B, Albiol J, Mattanovich D, Ferrer P. 2010. A multi-level study of recombinant Pichia pastoris in different oxygen conditions. BMC Syst. Biol. 4:141.
Cvijovic M, Olivares-Hernandez R, Agren R, Dahr N, Vongsangnak W, Nookaew I, Patil K R, Nielsen J. 2010. BioMet Toolbox: Genome-wide analysis of metabolism. Nucleic Acids Res. 38:144-149.
d'Anjou M C, Daugulis A J. 2000. Mixed-feed exponential feeding for fed-batch culture of recombinant methylotrophic yeast. Biotechnol. Lett. 22:341-346.
Dragosits M, Stadlmann J, Graf A, Gasser B, Maurer M, Sauer M, Kreil T R, Altmann F, Mattanovich D. 2010. The response to unfolded protein is involved in osmotolerance of Pichia pastoris: 1-16.
Gerngross T U. 2004. Advances in the production of human therapeutic proteins in yeasts and filamentous fungi. Nat. Biotechnol. 22:1409-1414.
Görgens JF, Van Zyl W H, Knoetze J H, Hahn-Hägerdal B. 2005. Amino acid supplementation improves heterologous protein production by Saccharomyces cerevisiae in defined medium. Appl. Microbiol. Biotechnol. 67:684-691.
Hellenbroich D, Valley U, Ryll T, Wagner R, Tekkanat N, Kessler W, Ross A, Deckwer W D. 1999. Cultivation of Tetrahymena thermophila in a 1.5-m3 airlift bioreactor. Appl. Microbiol. Biotechnol. 51:447-55.
Heyland J, Fu J, Blank L M, Schmid A. 2011. Carbon metabolism limits recombinant protein production in Pichia pastoris. Biotechnol. Bioeng. 108:1942-1953.
Invitrogen Corporation. 2002. Pichia Fermentation Process Guidelines Overview Overview: 1-11.
Liang S, Wang B, Pan L, Ye Y, He M, Han S, Zheng S, Wang X, Lin Y. 2012. Comprehensive structural annotation of Pichia pastoris transcriptome and the response to various carbon sources using deep paired-end RNA sequencing. BMC Genomics 13:738.
Love K R, Shah K A, Whittaker C A, Wu J, Bartlett M C, Ma D, Leeson R L, Priest M, Borowsky J, Young S K, Love J C. 2016. Comparative genomics and transcriptomics of Pichia pastoris. BMC Genomics 17:550.
Love M I, Huber W, Anders S. 2014. Moderated estimation of fold change and dispersion for RNA-seq data with DESeq2. Genome Biol. 15:550.
Olz R, Larsson K, Adler L, Gustafsson L. 1993. Energy flux and osmoregulation of Saccharomyces cerevisiae grown in chemostats under NaCl stress. J. Bacteriol. 175:2205-2213.
Pais-Chanfrau J M, Trujillo-Toledo L E. 2016. Optimization of culture medium for large-scale production of heterologous proteins in Pichia pastoris to be used in nanoscience and other biotechnological fields. Biol. Med. 8:8-11.
Prielhofer R, Cartwright S P, Graf A B, Valli M, Bill R M, Mattanovich D, Gasser B. 2015. Pichia pastoris regulates its gene-specific response to different carbon sources at the transcriptional, rather than the translational, level. BMC Genomics 16:1-17.
Schneider M, Marison I W, Von Stockar U. 1996. The importance of ammonia in mammalian cell culture. J. Biotechnol. 46:161-185.
Stratton J, Chiruvolu V, Meagher M. 1998. High cell-density fermentation. Methods Mol. Biol. 103:107-120.
Tomàs-Gamisans M, Ferrer P, Albiol J. 2016. Integration and Validation of the Genome-Scale Metabolic Models of Pichia pastoris: A Comprehensive Update of Protein Glycosylation Pathways, Lipid and Energy Metabolism. PLoS One 11.
van der Valk J, Brunner D, De Smet K, Fex Svenningsen A, Honegger P, Knudsen L E, Lindl T, Noraberg J, Price A, Scarino M L, Gstraunthaler G. 2010. Optimization of chemically defined cell culture media—Replacing fetal bovine serum in mammalian in vitro methods. Toxicol. Vitr. 24:1053-1063.
Verduyn C, Postma E, Scheffers W a., Van Dijken J P. 1992. Effect of benzoic acid on metabolic fluxes in yeasts: A continuous-culture study on the regulation of respiration and alcoholic fermentation. Yeast 8:501-517.
Wang J, Nguyen V, Glen J, Henderson B, Saul A, Miller L H. 2005. Improved yield of recombinant merozoite surface protein 3 (MSP3) from Pichia pastoris using chemically defined media. Biotechnol. Bioeng. 90:838-847.
Yang J, Zhou X, Zhang Y. 2004. Improvement of recombinant hirudin production by controlling NH4+ concentration in Pichia pastoris fermentation. Biotechnol. Lett. 26:1013-7.
Zhang J, Reddy J, Buckland B, Greasham R. 2003. Toward consistent and productive complex media for industrial fermentations: Studies on yeast extract for a recombinant yeast fermentation process. Biotechnol. Bioeng. 82:640-652.
Zhang W, Sinha J, Meagher M M. 2006. Glycerophosphate as a phosphorus source in a defined medium for Pichia pastoris fermentation. Appl. Microbiol. Biotechnol. 72:139-144.

### Example 2: Optimizing Amino Acids in Defined Media for Pichia pastoris Recombinant Protein Expression

### Introduction

In the biopharmaceutical industry, media for host organisms have been optimized with nutrients like amino acids to maximize recombinant protein production. Because amino acids serve as the building blocks of proteins, as well as intermediates in various metabolic pathways, they are posited to relieve much of the metabolic burden experienced by organisms producing heterologous proteins. Both complex amino acid mixtures and defined amino acids have been shown to improve protein titers in various organisms. While there have been some studies on media optimization with amino acids for yeast, few systematic studies of amino acid supplementation have been reported for Pichia pastoris, a widely-used organism for producing biological therapeutics. Commercially available defined media often lacks nutrients, including specific amino acids found in complex media. However, defined media has the potential to simplify downstream protein purification and quality control, and is therefore of great interest. Here, a study is presented of the impact of adding specific amino acids on recombinant protein titers. To uncover a general formulation, three P. pastoris strains were studied, each expressing a different heterologous protein. Across all three strains, some amino acids increased protein production, while some amino acids decreased protein production relative to controls lacking amino acids.

### Materials and Methods

Using P. pastoris in Defined Media for Biopharmaceutical Production

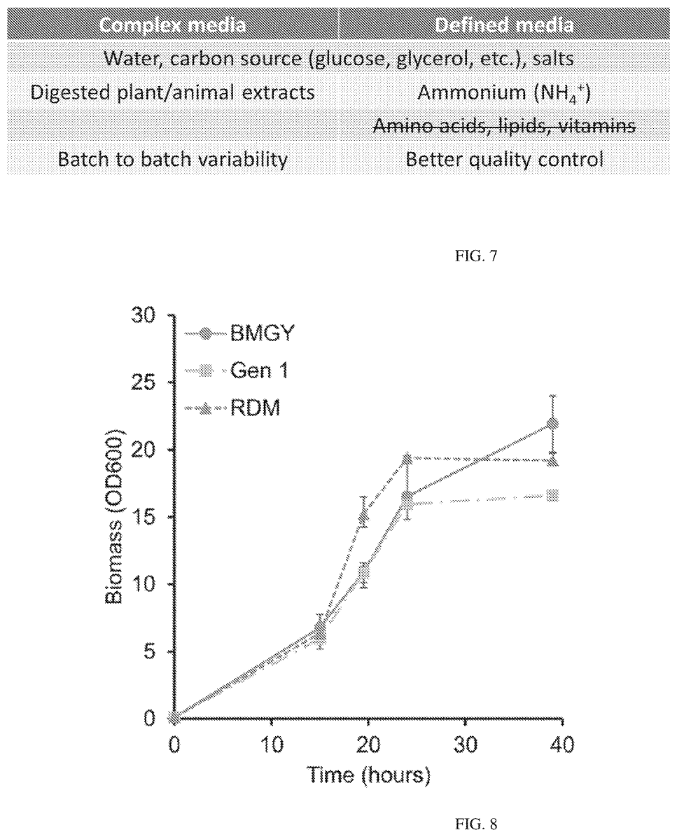

Defined media are less explored than complex media for P. pastoris. Though complex media contain nutrients important for cell growth and protein expression, the exact composition is unknown so defined media is preferred for quality control. However, common defined media for P. pastoris only contain minimal salts and lack the nutrients found in complex media. These nutrients are important for increasing cell growth and productivity. The difference between complex and defined media can be seen in FIG. 7.

Development of Enriched Defined Medium for P. pastoris Growth Also LED to Increase in Protein Expression

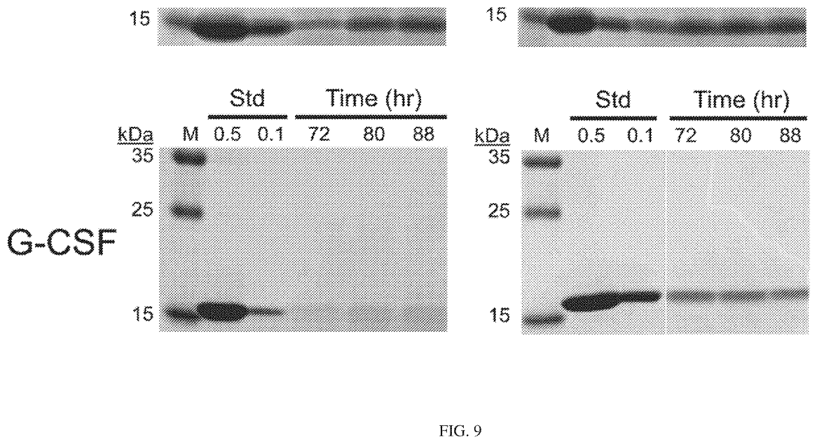

Usually, cell growth is slower in minimal defined media than in the nutrient-rich buffered glycerol complex media (BMGY). However in a previous study, cells grown in the rich defined medium (RDM) grew more quickly than in BMGY and reached similar biomasses (FIG. 8). Recombinant protein expression is also usually lower in minimal defined media than in complex media but protein expression in RDM surpasses that in BMGY (FIG. 9). Given the improvements in growth and protein expression with RDM, there is motivation to further improve protein titers using amino acid supplementation.

### Results and Discussion

### Impact of Individual Amino Acids on Protein Expression

Some amino acids consistently increase protein expression and some amino acids decrease protein expression relative to control with no amino acids. Expression of three recombinant proteins, hGH, G-CSF, and IFN, by P. pastoris grown in media with different individual amino acids, 12.5 mM each. Protein concentrations measured by GXII, samples taken 24 hours after methanol induction (FIG. 10).

Combinations of Amino Acids Vs. Individual Amino Acids

Combinations of amino acids based on individual amino acid screening were not necessarily as beneficial as individual amino acids. (FIG. 11). hGH expression by P. pastoris grown in media with different combinations of amino acids, each 12.5 mM total, based on initial individual amino acid screenings is shown. hGH concentrations were measured by GXII, samples taken 24 hours after methanol induction.

### Starred Media Conditions are Media Formulations of Interest

In FIG. 12, the effect of Asparagine, Glutamine, and Arginine on protein expression is examined. The starred media conditions are as follows; Glutamine, Arginine, Glutamine+Arginine (RDM), Asparagine, Glutamine+Asparagine, and all 20 amino acids. In all media conditions, total amino acid concentration is normalized to 25 mM.

### Amino Acid Titers

hGH production was measured in media containing individual or combinations of amino acids. The total concentration in (mM) of amino acids was 12.5, 25, or 50 mM. The results were measured by ELISA, and each value is the average of three technical replicates. It was found that 25 mM amino acid media seem to result in the highest protein titers (FIG. 13).

### Reference

Matthews, C. B., Kuo, A., Love, K. R., and Love, J. C. (2017). Development of a general defined medium for Pichia pastoris. Biotechnol Bioeng.

### Example 3: Implications of Methanol-Induced Remodeling of Gene Expression for P. pastoris Process Design

### Introduction

The use of Pichia pastoris as an expression system is built around the concept of strongly inducible protein production (Potvin et al., 2012). Heterologous proteins are expressed under the control of the pAOX1 promoter, which is induced when cells are grown on methanol. Cultivations are conducted in two phases: a biomass accumulation phase with glycerol as the carbon source followed by a protein production phase with methanol as the carbon source. General carbon feeding strategies have been developed based on kinetic growth models, but input parameters have to be determined empirically for each strain (Looser et al., 2014). Understanding how these input parameters relate to the biological state of P. pastoris while growing on methanol is critical to increasing the productivity of fermentation processes.

Gene expression analysis is a powerful tool to study effects of experimental conditions on biological state, and the falling costs of RNA-Seq have made it feasible to routinely sequence expression across the whole genome (Wang et al., 2009). In P. pastoris, transcriptomic differences between growth on methanol and on conventional carbon sources glycerol and glucose have been characterized for cells grown in batch shake flasks (Liang et al., 2012; Love et al., 2016; Prielhofer et al., 2015), in fed-batch bioreactors (Edwards-Jones et al., 2015), and in chemostat reactors (Rußmayer et al., 2015). The findings have been used to inform strain development objectives, such as identification of new promoters, but have not been applied to fermentation process design. Transcriptomic understanding of biological states could accelerate the development of highly productive perfusion processes, which have not yet been developed for this organism.

In this work, functional analysis of gene expression over time during a perfusion cultivation was used to identify areas for process improvement. Based on expression of genes involved in nutrient limitation and protein folding and secretion, a decline in cellular health was characterized and attributed to carbon source limitation. When methanol feed rate was increased, the highest-achieved cell density increased by a factor of four and the maximum heterologous protein titer increased by a factor of six. Based on these findings, the iterative application of gene expression analysis is recommended to inform further optimization of the feeding of methanol and other carbon sources.

### Methods

### Cultivation Methods

Experiments were conducted using a K. phaffii strain modified to express human growth hormone (hGH) under the control of the AOX1 promoter. For biomass accumulation, cells were grown in medium containing per liter: 40 mL glycerol, 12 g KH₂PO₄, 4.7 g MgSO₄.7H₂O, 0.36 g CaCl₂.2H₂O, 4.96 g (NH₄)₂SO₄, 1.46 g L-glutamine, 1.74 g L-arginine, 3.3 mL of vitamin solution, 10 mL of lipids solution, and 4.35 mL of PTM1 salts (Amresco J241). For protein production, cells were grown in medium containing per liter: 12 g KH₂PO₄, 4.7 g MgSO₄.7H₂O, 0.36 g CaCl₂.2H₂O, 13.21 g (NH₄)₂SO₄, 2.92 g L-glutamine, 3.3 mL of vitamin solution, 10 mL of lipids solution, and 4.35 mL of PTM1 salts (Amresco J241). Both media were adjusted to pH 6.5 using 5M KOH. The vitamin solution consisted of per liter: 7.5 g myo-inositol, 0.3 g thiamine HCl, 0.3 g calcium pantothenate, 0.3 g nicotinic acid, 0.3 g pyroxidine HCl, 0.015 g biotin, and 0.06 g para-aminobenzoic acid. The lipids solution consisted of per liter: 2 mg arachidonic acid, 10 mg linoleic acid, 10 mg linolenic acid, 10 mg myristic acid, 10 mg oleic acid, 10 mg palmitic acid, 10 mg stearic acid, 2.2 g Tween-80, 70 mg tocopherol acetate, and 100 g of Pluronic F-68. All chemicals were purchased from Sigma. The medium was filtered before use.

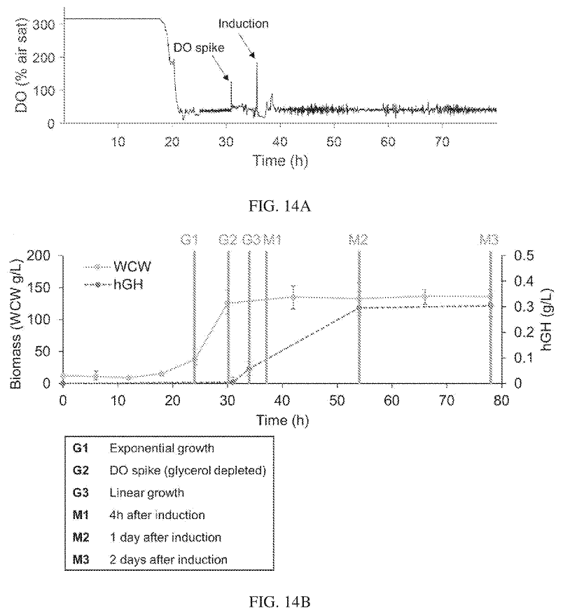

Cultivations were conducted in 0.5 L Infors HT bioreactors adapted for operation in perfusion mode. Cultures were inoculated directly from a frozen cell stock into 0.44 L of medium. Cells were grown for approximately 32 hours in perfusion mode with medium containing glycerol fed at a rate of 0.5 mL/minute. A dissolved oxygen (DO) spike was observed when the glycerol feed became limiting (FIG. 14A). The cultures were induced by switching the feed to production medium containing methanol, which was also fed at 0.5 mL/minute. Bioreactor temperature was automatically controlled at 25° C. The pH was controlled at 6.5 using 5M KOH. Dissolved oxygen was maintained at 40% of air saturation by the use of split-range control. Pure oxygen was initially sparged in a range of 0.04-0.2 L/min and agitation was subsequently increased from 500-1500 RPM.

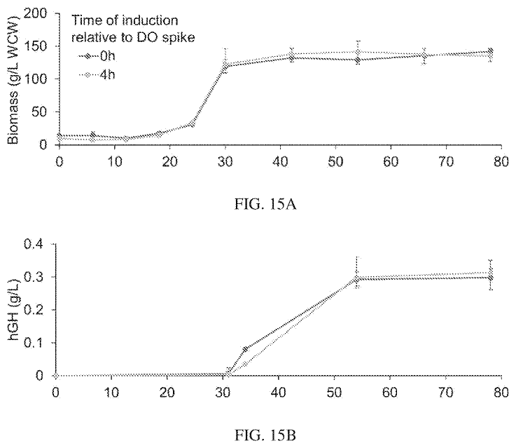

To study gene expression under perfusion, seven cultivations were conducted (FIG. 14B). The production medium contained 1.5% v/v methanol. For four of the bioreactors (labeled 1, 3, 5A, 6A), the cultures were induced immediately after the DO spike. For the other three reactors (labeled 4, 5, 6), glycerol feed was maintained after the DO spike for 4 hours and then the cultures were induced with methanol. There were no significant differences between the biomass, hGH titers, or transcriptomic data between the two conditions (FIG. 15), so they were treated as biological replicates for RNA-Seq analysis.

RNA was isolated from samples taken at six time points during the cultivation (FIG. 14B). Samples were taken from all reactors after one day of exponential growth on glycerol (G1) and immediately after the DO spike (G2). For the three bioreactors for which glycerol feed was maintained for 4 hours after the DO spike, samples were taken at the end of this period (G3). For the other four bioreactors, samples were taken after 4 hours of methanol feeding (M1). Samples were taken from all bioreactors one day (M2) and two days (M3) after the start of methanol feeding.

For the studies of higher methanol feed rate, two cultivations were conducted. The production medium contained 15% v/v methanol. All other fermentation conditions were as described above.

Biomass was measured by wet cell weight. Five hundred microliters of sample were spun in a Costar Spin-X centrifuge filter tube for 10 minutes at 1500 g and the pellet was weighed. hGH concentrations in the supernatants were quantified by RPLC and by gel electrophoresis (Perkin Elmer GXII). Concentrations estimated by the two methods were highly consistent (slope=0.98, R²=0.95).

Cell-specific productivity was calculated according to the following system of equations (Amos Lu, unpublished):

$$\frac{d X}{dt} = μ X , \\ \frac{d P}{dt} = {q}_{p} X - PF , \\ {q}_{p} = \frac{μ + F}{{X}_{0}} ( \frac{P - {P}_{0} exp [ - F t ]}{exp [ μ t ] - exp [ - F t ]} ) ,$$

where qₚ is the cell-specific productivity, μ is the growth rate, F is the perfusion rate, X is the biomass concentration, and P is the protein titer.

### Transcriptome Analysis

RNA was extracted using the Qiagen RNeasy kit (cat. no. 74104). Libraries for the batch cultivations were constructed using the Truseq mRNA stranded HT kit. Libraries for the bioreactor cultivations were constructed using Neoprep. All libraries were sequenced on the Illumina NextSeq platform to generate 75-nucleotide paired-end reads at a read depth of at least 3 million reads per sample. The reads were aligned to the previously reported genome sequence for this strain (Love et al., 2016) using RSEM version 1.2.15 with bowtie2 version 2.2.3. The reads were also aligned to the sequence for hGH using BWA (version 0.7.12).

To assess the technical quality of RNA-seq reads for each condition sampled, each raw data set was down-sampled to IM paired-end reads and aligned to the assembly using BWA 0.7.5a. Bedtools (version 2.17.0) was used to overlap the resulting alignments to the annotations to count the reads falling into genes, intronic regions, 5′ or 3′ UTRs, flanking 3 kb genic regions and intergenic regions. Other basic statistics, including number of reads, mapping rate, ratio of sense vs. anti-sense reads, and rRNA rate were also collected for each sample (Table 6).

The complete data set for each condition and time point was used for all analyses reported. Unsupervised hierarchical clustering (Ward's Method) implemented in Spotfire 7.6.1 was used to examine data relationships for the biological replicates performed for each cultivation condition. Consistent clustering of biological replicates was observed. Count data for analysis of differential expression and ranking by the Wald statistic was done using DESeq2 in R version 3.2.3 (Love et al., 2014). Single Sample Gene Set Enrichment Analysis (ssGSEA) version 8 was conducted using GenePattern 2.0 (Reich et al., 2006; Subramanian et al., 2005) with previously reported biological process gene sets for P. pastoris (Love et al., 2016).

TABLE 6
Quality control metrics for RNA-sequencing data from perfusion cultivations.

| | | | # | | | | flanking | inter- | Reads | Mapped | sense/ | rRNA |
|---|---|---|---|---|---|---|---|---|---|---|---|---|
| Rep | Time | Desc. | CDS | UTR5 | UTR3 | Intron | (+3k) | genic | (M) | % | antisense | % |
| 1 | 24 h | G1 | 92.5% | 1.0% | 2.1% | 0.03% | 4.6% | 0.04% | 29.3 | 98.3% | 3.4% | 0.19% |
| 3 | 24 h | G1 | 92.6% | 1.0% | 2.1% | 0.03% | 4.5% | 0.04% | 28.1 | 98.0% | 3.4% | 0.19% |
| 4 | 24 h | G1 | 92.8% | 1.0% | 2.0% | 0.03% | 4.4% | 0.04% | 20.4 | 97.8% | 3.3% | 0.18% |
| 5 | 24 h | G1 | 93.4% | 0.9% | 1.8% | 0.03% | 4.1% | 0.05% | 21.1 | 97.8% | 3.2% | 0.21% |
| 5A | 24 h | G1 | 92.9% | 0.9% | 2.0% | 0.03% | 4.4% | 0.04% | 23.0 | 96.1% | 3.5% | 0.17% |
| 6 | 24 h | G1 | 92.8% | 1.0% | 1.9% | 0.03% | 4.5% | 0.04% | 28.8 | 97.5% | 3.3% | 0.16% |
| 6A | 24 h | G1 | 92.0% | 1.0% | 2.4% | 0.03% | 4.8% | 0.04% | 19.5 | 96.9% | 4.1% | 0.19% |
| 1 | 30 h | G2 | 91.5% | 1.1% | 2.2% | 0.04% | 5.3% | 0.19% | 19.3 | 95.9% | 4.9% | 0.87% |
| 3 | 30 h | G2 | 89.4% | 1.7% | 2.7% | 0.05% | 6.5% | 0.08% | 14.8 | 96.1% | 5.4% | 0.40% |
| 4 | 30 h | G2 | 93.1% | 0.8% | 1.7% | 0.05% | 4.4% | 0.06% | 17.7 | 96.6% | 4.0% | 0.30% |
| 5 | 30 h | G2 | 90.8% | 1.4% | 2.1% | 0.04% | 5.8% | 0.14% | 13.1 | 93.4% | 4.8% | 0.64% |
| 5A | 30 h | G2 | 92.8% | 0.9% | 1.6% | 0.04% | 4.8% | 0.06% | 18.6 | 94.9% | 4.5% | 0.24% |
| 6 | 30 h | G2 | 89.4% | 1.6% | 2.8% | 0.05% | 6.4% | 0.11% | 10.9 | 94.0% | 5.6% | 0.49% |
| 6A | 30 h | G2 | 93.8% | 0.8% | 1.2% | 0.04% | 4.2% | 0.03% | 19.5 | 96.4% | 3.6% | 0.12% |
| 4 | 34 h | G3 | 91.6% | 1.0% | 2.9% | 0.04% | 4.9% | 0.05% | 16.7 | 95.9% | 13.1% | 0.18% |
| 5 | 34 h | G3 | 92.2% | 0.8% | 2.1% | 0.03% | 5.1% | 0.03% | 24.5 | 97.3% | 4.0% | 0.12% |
| 6 | 34 h | G3 | 92.5% | 0.8% | 2.1% | 0.04% | 4.8% | 0.03% | 24.1 | 96.9% | 3.9% | 0.12% |
| 1 | 34 h | M1 | 93.6% | 0.9% | 1.5% | 0.04% | 4.1% | 0.10% | 16.9 | 92.2% | 6.5% | 0.43% |
| 3 | 34 h | M1 | 93.7% | 0.9% | 1.6% | 0.03% | 4.0% | 0.09% | 20.0 | 94.8% | 6.7% | 0.34% |
| 5A | 34 h | M1 | 93.9% | 0.8% | 1.4% | 0.03% | 4.0% | 0.06% | 22.2 | 95.5% | 4.3% | 0.21% |
| 6A | 34 h | M1 | 93.0% | 0.8% | 2.2% | 0.03% | 4.2% | 0.02% | 17.5 | 95.2% | 5.7% | 0.06% |
| 1 | 54 h | M2 | 93.1% | 0.8% | 2.1% | 0.05% | 4.1% | 0.03% | 17.7 | 88.0% | 4.3% | 0.13% |
| 3 | 54 h | M2 | 93.5% | 0.7% | 2.1% | 0.04% | 3.7% | 0.02% | 20.3 | 88.7% | 4.0% | 0.10% |
| 4 | 54 h | M2 | 93.5% | 0.7% | 2.3% | 0.04% | 3.6% | 0.02% | 18.0 | 88.4% | 4.7% | 0.10% |
| 5 | 54 h | M2 | 93.9% | 0.7% | 2.0% | 0.05% | 3.6% | 0.03% | 16.9 | 86.4% | 3.7% | 0.15% |
| 5A | 54 h | M2 | 95.0% | 0.7% | 1.2% | 0.04% | 3.2% | 0.03% | 16.8 | 87.1% | 3.3% | 0.11% |
| 6 | 54 h | M2 | 93.7% | 0.7% | 2.0% | 0.04% | 3.7% | 0.02% | 20.7 | 90.0% | 3.6% | 0.10% |
| 6A | 54 h | M2 | 93.8% | 0.7% | 1.5% | 0.04% | 4.0% | 0.21% | 12.7 | 87.0% | 4.2% | 0.90% |
| 1 | 78 h | M3 | 94.7% | 0.6% | 1.4% | 0.04% | 3.3% | 0.04% | 17.9 | 84.1% | 3.4% | 0.19% |
| 3 | 78 h | M3 | 93.4% | 0.7% | 2.2% | 0.03% | 3.8% | 0.04% | 19.2 | 92.0% | 4.1% | 0.19% |
| 4 | 78 h | M3 | 94.0% | 0.7% | 1.8% | 0.04% | 3.6% | 0.04% | 13.5 | 91.7% | 3.6% | 0.19% |
| 5 | 78 h | M3 | 93.1% | 0.7% | 2.6% | 0.04% | 3.7% | 0.03% | 15.2 | 89.9% | 3.8% | 0.15% |
| 5A | 78 h | M3 | 92.5% | 0.8% | 3.0% | 0.02% | 3.9% | 0.04% | 15.7 | 91.4% | 4.9% | 0.16% |
| 6 | 78 h | M3 | 92.8% | 0.7% | 2.5% | 0.04% | 4.1% | 0.09% | 15.4 | 90.1% | 4.2% | 0.46% |
| 6A | 78 h | M3 | 93.5% | 0.7% | 1.7% | 0.04% | 3.9% | 0.27% | 13.5 | 89.7% | 4.5% | 1.28% |

### Results and Discussion

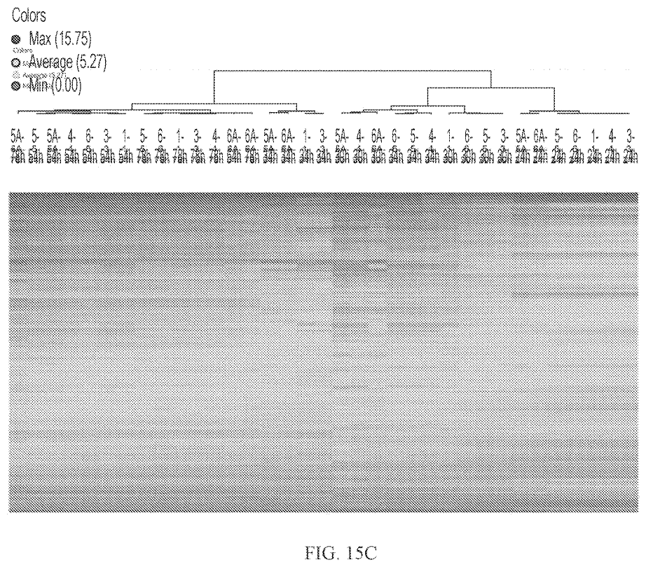

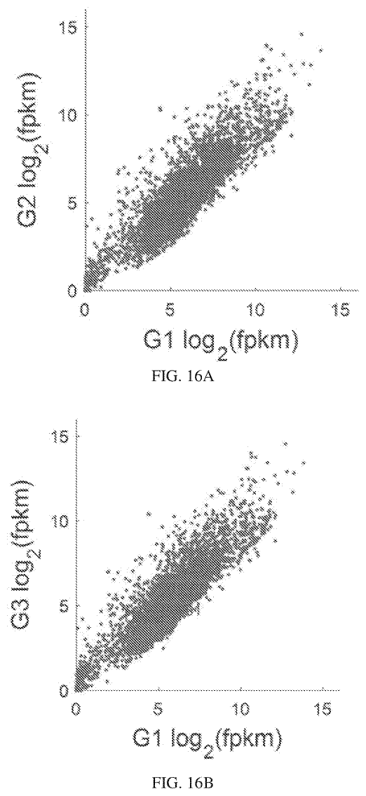

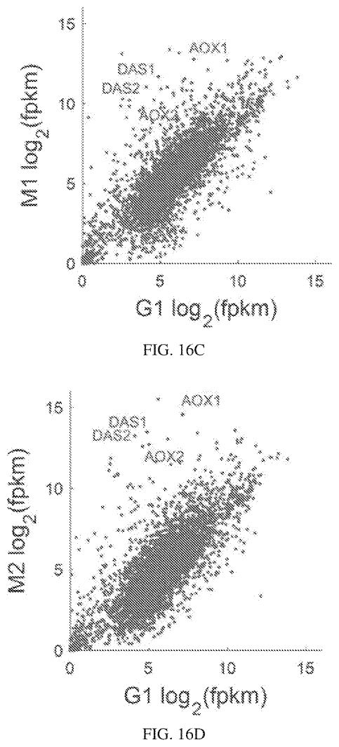

The gene expression was first characterized across the whole genome at different stages of perfusion cultivation (FIGS. 16A-16E). Across the glycerol-feeding states, a distinct difference in gene expression is observed between G1, when the cells were grown on excess glycerol, and G2 and G3, when glycerol was fed at a growth-limiting rate (R²=0.76, 0.77). After induction with methanol, gene expression continued to change relative to G1 but was similar across the three methanol feeding points M1, M2, and M3 (R²=0.64, 0.61, 0.63). The expression of genes involved in methanol metabolism increased by 35-fold in the first 4 hours and later to 100-fold higher than the expression level on glycerol.

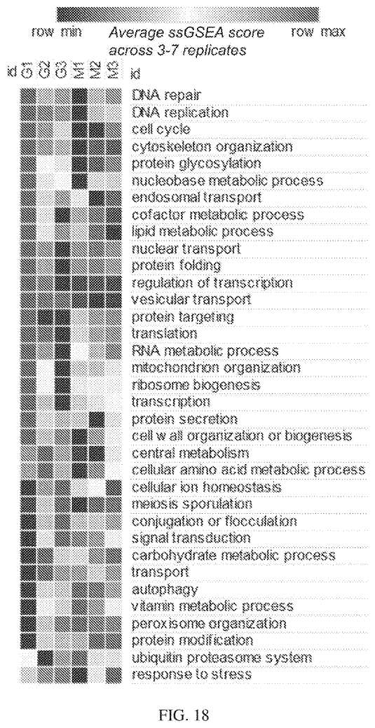

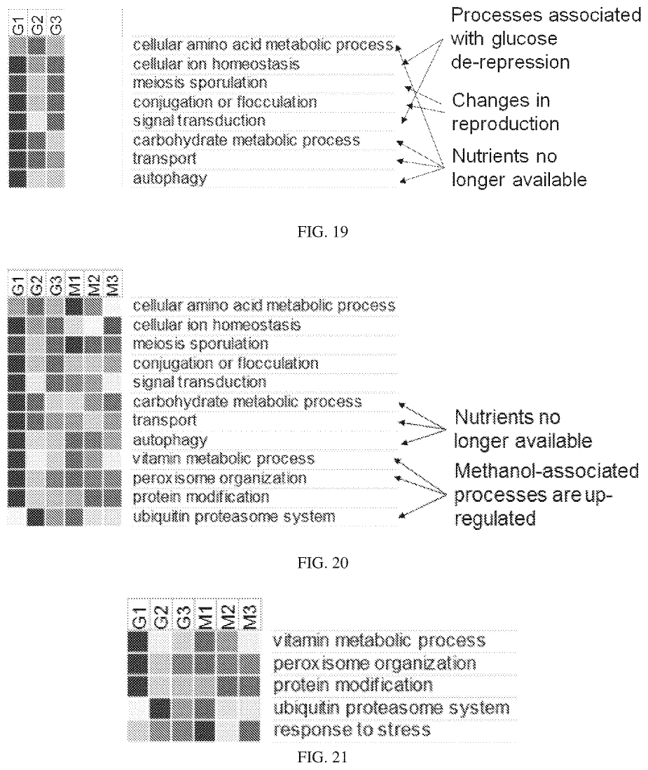

ssGSEA was used to provide insight into changes in biological processes, which clustered into co-expressed groups (FIGS. 17-21). In the exponential growth state (G1), the cells displayed the highest expression of genes involved in DNA replication and repair, cell cycle, and central metabolism. Previous studies suggest that these processes are most highly expressed when the growth rate is high (Edwards-Jones et al., 2015). In states G2 and G3, when glycerol was limited, significant changes in biological state occurred. The lower carbon availability triggered changes in reproduction, reflected by up-regulation of meiosis, sporulation, conjugation, and flocculation. The previously identified growth-associated processes were down-regulated and expression increased in processes that reflect reduced nutrient availability including autophagy, transport, carbohydrate metabolism, and amino acid metabolism. Processes that have been reported to be associated with glucose de-repression, such as ion homeostasis and signal transduction (Alepuz et al., 1997; Thevelein, 1994), were also up-regulated. Despite the consistent rate of glycerol feeding, the cell-specific carbon feed rate fell as biomass accumulated and that reduction had significant impacts on the cells' biological state.

Methanol is known to be a difficult carbon source to metabolize, so whether signs of nutrient limitation would persist once methanol feeding began was investigated. Autophagy, transport, and carbohydrate metabolism remained up-regulated, while central metabolism and amino acid metabolism were no longer as highly expressed. Consistent with previous reports in other formats, methanol feeding also resulted in significant up-regulation of peroxisome organization and vitamin metabolism (Edwards-Jones et al., 2015; Liang et al., 2012). Neither of these is surprising: methanol is metabolized in the peroxisome and a thiamine derivative, TPP, is a co-factor of DAS1 and DAS2 (Rulmayer et al., 2015). These enzymes catalyze the conversion of formaldehyde, which is generated through the oxidation of methanol, to glyceraldehyde-3P, an intermediate in glycolysis.

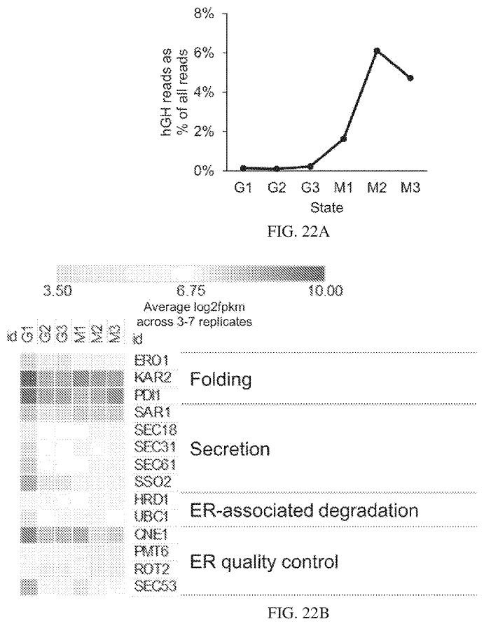

The transcriptomic states at M2 and M3 were expected to be similar because the methanol feed rate, biomass concentration, and protein titer are all the same. Expression of the hGH gene, however, dropped by over 40% (FIG. 22A) between M2 and M3 despite consistent expression of methanol metabolism genes (FIGS. 16A-16E). Additionally, vitamin metabolism is down-regulated and response to stress is up-regulated in M3 compared to M2. Within the stress response gene set, eight of the ten most differentially-expressed genes are specifically involved in oxidative or hydrogen peroxide stress response: HCM1, SRX1, STB5, TRR1, GSH1, ALO1, YAR1, and NCL1. While the effects are not yet visible in the titers, these transcriptomic data suggest that the cell state is in decline.

Typically reported signatures of heterologous protein stress are also not evident in the present transcriptomic data. Protein folding and secretion are well-established bottlenecks for recombinant protein production in P. pastoris (Love et al., 2012; Mattanovich et al., 2004) and previous transcriptomic studies demonstrate consistent up-regulation of ubiquitin-mediated proteolysis when methanol is fed (Edwards-Jones et al., 2015; Liang et al., 2012). In the present process, however, ubiquitin-mediated proteolysis is only significantly up-regulated in M1 and its expression falls as methanol feeding continues. Expression of genes specifically involved in protein folding and secretion (Gasser et al., 2007) does not increase on methanol feeding compared to glycerol (FIG. 22B). Given this data and the decline discussed above, it was hypothesized that protein production was limited by carbon source availability rather than protein folding and secretion stress.

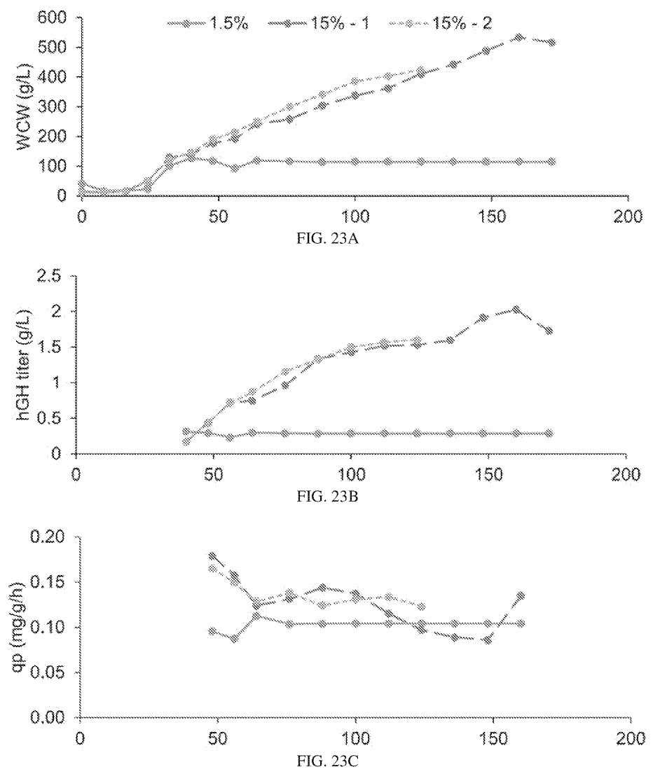

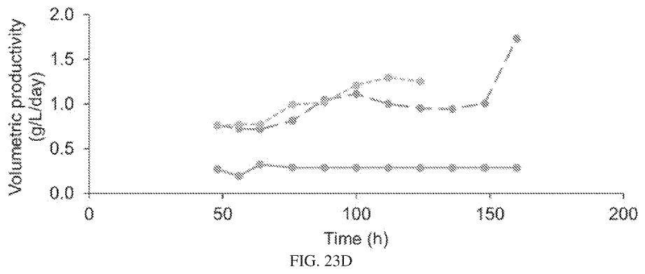

To test this hypothesis, the methanol feed rate was increased by a factor of ten. The cells grew at an average growth rate of 0.01 h⁻¹, reaching a biomass concentration of 530 g/L WCW on day 6 of production (FIG. 23A). This cell density was over four times greater that of the low-methanol feeding experiments and is the maximum cell density that can be maintained in the current reactor setup. The hGH titers were also over six times higher than those achieved at the lower methanol feed rate, as high as 2 g/L at the end of the cultivation (FIG. 23B). As suggested by these maximum values, cell-specific productivities were also higher at the higher methanol feed rate (FIG. 23C), double the low-feed case at the beginning of the cultivation. This finding indicates that the cells' biological states were more favorable for production. Volumetric productivities were also 3-4 times higher on average when more methanol was fed (FIG. 23D). These results demonstrate that significantly higher productivity can be achieved through changes in feeding and support the hypotheses about nutrient limitation that were generated from the transcriptomic data.

TABLE 7
Productivities of hGH in batch at different initial cell
densities. Experiments were conducted in shake flasks.

| Flask | 2x | 1x | 0.5x | 0.25x |
|---|---|---|---|---|
| Biomass at induction (OD₆₀₀) | 53.8 | 26.9 | 13.5 | 6.7 |
| mL MeOH | 3 | 1.5 | 0.75 | 0.38 |
| Biomass at harvest (OD₆₀₀) | 61.8 | 30.6 | 21.4 | 11.8 |
| hGH at harvest (ug/mL) | 102.8 | 53.7 | 21.5 | 12.9 |
| hGH/biomass (ug/OD₆₀₀) | 1.9 | 2.0 | 1.6 | 1.9 |

### Conclusions

The first transcriptomic characterization of a perfusion cultivation of Pichia pastoris was performed, providing new understanding of the differences in gene expression as cells were fed excess glycerol, limiting glycerol, and methanol. Biological processes were identified that were consistently up-regulated in all nutrient-limited states and others that were unique to methanol. Based on an observed decline in cellular health and an understanding of the activity of protein folding and secretion pathways, it was hypothesized that the carbon feeding was insufficient. When the methanol feeding rate was increased by a factor of ten, cell-specific productivities doubled and the highest achievable heterologous protein titer increased by a factor of six.

Further optimization of carbon feeding rates, informed by additional transcriptomic analysis to enable correlation of growth rate, cell-specific productivity, and gene expression profile, is possible. Once optimal feeding profiles are identified, productivities can be further increased by increasing operating cell densities. This work continues to demonstrate the usefulness of gene expression for process design, here for carbon sources specifically rather than the wide range of nutrients considered in the previous chapters.

### References

Adelantado N, Tarazona P, Grillitsch K, Garcia-Ortega X, Monforte S, Valero F, Feussner I, Daum G, Ferrer P. 2017. The effect of hypoxia on the lipidome of recombinant Pichia pastoris. Microb. Cell Fact. 16.
Alepuz P M, Cunningham K W, Estruch F. 1997. Glucose repression affects ion homeostasis in yeast through the regulation of the stress-activated ENA1 gene. Mol. Microbiol. 26:91-98.
Clare J J, Romanes M A, Rayment F B, Rowedder J E, Smith M A, Payne M M, Sreekrishna K, Henwood C A. 1991. Production of mouse epidermal growth factor in yeast: high-level secretion using Pichia pastoris strains containing multiple gene copies. Gene 105:205-212.
Clincke M F, Mlleryd C, Zhang Y, Lindskog E, Walsh K, Chotteau V. 2013. Very high density of CHO cells in perfusion by ATF or TFF in WAVE bioreactor. Part I: Effect of the cell density on the process. Biotechnol. Prog. 29:754-767.
Conesa A, Madrigal P, Tarazona S, Gomez-Cabrero D, Cervera A, McPherson A, Szczesniak M W, Gaffney D J, Elo L L, Zhang X, Mortazavi A. 2016. A survey of best practices for RNA-seq data analysis. Genome Biol. 17:13.
Cvijovic M, Olivares-Hernandez R, Agren R, Dahr N, Vongsangnak W, Nookaew I, Patil K R, Nielsen J. 2010. BioMet Toolbox: Genome-wide analysis of metabolism. Nucleic Acids Res. 38:144-149.
Edwards-Jones B, Aw R, Barton G R, Tredwell G D, Bundy J G, Leak D J. 2015. Translational arrest due to cytoplasmic redox stress delays adaptation to growth on methanol and heterologous protein expression in a typical fed-batch culture of Pichia pastoris. PLoS One 10:1-25.
Gasser B, Sauer M, Maurer M, Stadlmayr G, Mattanovich D. 2007. Transcriptomics-based identification of novel factors enhancing heterologous protein secretion in yeasts. Appl. Environ. Microbiol. 73:6499-6507.
Görgens JF, Van Zyl W H, Knoetze J H, Hahn-Hägerdal B. 2005. Amino acid supplementation improves heterologous protein production by Saccharomyces cerevisiae in defined medium. Appl. Microbiol. Biotechnol. 67:684-691.
Hellwig S, Emde F, Raven N P G, Henke M, Van Logt P Der, Fischer R. 2001. Analysis of single-chain antibody production in Pichia pastoris using on-line methanol control in fed-batch and mixed-feed fermentations. Biotechnol. Bioeng. 74:344-352.
Heyland J, Fu J, Blank L M, Schmid A. 2011. Carbon metabolism limits recombinant protein production in Pichia pastoris. Biotechnol. Bioeng. 108:1942-1953.
Hiltunen J K, Mursula A M, Rottensteiner H, Wierenga R K, Kastaniotis A J, Gurvitz A. 2003. The biochemistry of peroxisomal 3-oxidation in the yeast Saccharomyces cerevisiae. FEMS Microbiol. Rev. 27:35-64.
Jungo C, Schenk J, Pasquier M, Marison I W, von Stockar U. 2007. A quantitative analysis of the benefits of mixed feeds of sorbitol and methanol for the production of recombinant avidin with Pichia pastoris. J. Biotechnol. 131:57-66.
Liang S, Wang B, Pan L, Ye Y, He M, Han S, Zheng S, Wang X, Lin Y. 2012. Comprehensive structural annotation of Pichia pastoris transcriptome and the response to various carbon sources using deep paired-end RNA sequencing. BMC Genomics 13:738.
Looser V, Bruhlmann B, Bumbak F, Stenger C, Costa M, Camattari A, Fotiadis D, Kovar K. 2014. Cultivation strategies to enhance productivity of Pichia pastoris: A review. Biotechnol. Adv. 33:1177-1193.
Love K R, Shah K A, Whittaker C A, Wu J, Bartlett M C, Ma D, Leeson R L, Priest M, Borowsky J, Young S K, Love J C. 2016. Comparative genomics and transcriptomics of Pichia pastoris. BMC Genomics 17:550.
Love M I, Huber W, Anders S. 2014. Moderated estimation of fold change and dispersion for RNA-seq data with DESeq2. Genome Biol. 15:550.
Mattanovich D, Gasser B, Hohenblum H, Sauer M. 2004. Stress in recombinant protein producing yeasts. J. Biotechnol. 113:121-135.
Niu H, Jost L, Pirlot N, Sassi H, Daukandt M, Rodriguez C, Fickers P. 2013. A quantitative study of methanol/sorbitol co-feeding process of a Pichia pastoris Mut+/pAOX1-lacZ strain. Microb. Cell Fact. 12:33.
Ogata H, Goto S, Sato K, Fujibuchi W, Bono H, Kanehisa M. 1999. KEGG: Kyoto encyclopedia of genes and genomes. Nucleic Acids Res. 27:29-34.
Patil K R, Nielsen J. 2005. Uncovering transcriptional regulation of metabolism by using metabolic network topology. Proc Natl Acad Sci USA 102:2685-2689.
Potvin G, Ahmad A, Zhang Z. 2012. Bioprocess engineering aspects of heterologous protein production in Pichia pastoris: A review. Biochem. Eng. J. 64:91-105.
Prielhofer R, Cartwright S P, Graf A B, Valli M, Bill R M, Mattanovich D, Gasser B. 2015. Pichia pastoris regulates its gene-specific response to different carbon sources at the transcriptional, rather than the translational, level. BMC Genomics 16:1-17.
Reich M, Liefeld T, Gould J, Lerner J, Tamayo P, Mesirov J P. 2006. GenePattern 2.0. Nat. Genet. 38:500-501.
Rußmayer H, Buchetics M, Gruber C, Valli M, Grillitsch K, Modarres G, Guerrasio R, Klavins K, Neubauer S, Drexler H, Steiger M, Troyer C, Al Chalabi A, Krebiehl G, Sonntag D, Zellnig G, Daum G, Graf A B, Altmann F, Koellensperger G, Hann S, Sauer M, Mattanovich D, Gasser B. 2015. Systems-level organization of yeast methylotrophic lifestyle. BMC Biol. 13:80.
Subramanian A, Tamayo P, Mootha V K, Mukherjee S, Ebert B L, Gillette M A, Paulovich A, Pomeroy S L, Golub T R, Lander E S, Mesirov J P. 2005. Gene set enrichment analysis: a knowledge-based approach for interpreting genome-wide expression profiles. Proc. Natl. Acad. Sci. U.S.A. 102:15545-50.
The Gene Ontology Consortium. 2015. Gene ontology consortium: Going forward. Nucleic Acids Res. 43:D1049-D1056.
Thevelein J M. 1994. Signal transduction in yeast. Yeast 10:1753-1790.
Thorpe E D, D'Anjou M C, Daugulis A J. 1999. Sorbitol as a non-repressing carbon source for fed-batch fermentation of recombinant Pichia pastoris. Biotechnol. Lett. 21:669-672.
Tomàs-Gamisans M, Ferrer P, Albiol J. 2016. Integration and Validation of the Genome-Scale Metabolic Models of Pichia pastoris: A Comprehensive Update of Protein Glycosylation Pathways, Lipid and Energy Metabolism. PLoS One 11.
Wang J, Nguyen V, Glen J, Henderson B, Saul A, Miller L H. 2005. Improved yield of recombinant merozoite surface protein 3 (MSP3) from Pichia pastoris using chemically defined media. Biotechnol. Bioeng. 90:838-847.
Wang Z, Gerstein M, Snyder M. 2009. RNA-Seq: a revolutionary tool for transcriptomics. Nat. Rev. Genet. 10:57-63.

### EQUIVALENTS

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

All references, patents and patent applications that are recited in this application are incorporated by reference herein in their entirety.

1. A cell culture medium comprising:
(a) KH₂PO₄;
(b) MgSO₄;
(c) CaCl₂; and
(d) one or more of:
(i) ammonium at a concentration of 0.2-3.7 g/L;
(ii) one or more of biotin, calcium pantothenate, nicotinic acid, inositol, thiamin HCl, pyridoxine HCl, and para-aminobenzoic acid;
(iii) glutamine;
(iv) arginine; and
(e) one or more of arachidonic fatty acid, linoleic fatty acid, linolenic fatty acid, myristic fatty acid, oleic fatty acid, palmitic fatty acid, stearic fatty acid, and cholesterol, wherein the g/L denote concentration when the medium is diluted in a solvent.

2-5. (canceled)

6. The medium of claim 1, comprising glutamine at a concentration of ≤15 g/L, wherein the g/L denote concentration when the medium is diluted in a solvent.

7. The medium of claim 1, comprising arginine at a concentration of ≤15 g/L, wherein the g/L denote concentration when the medium is diluted in a solvent.

8-28. (canceled)

29. The medium of claim 1, comprising 2, 3, 4, 5, 6, 7, or all of arachidonic fatty acid, linoleic fatty acid, linolenic fatty acid, myristic fatty acid, oleic fatty acid, palmitic fatty acid, stearic fatty acid, and cholesterol.

30. The medium of claim 1, comprising one or more of:
arachidonic fatty acid at a concentration of 5 μg/L-1 g/L, optionally at a concentration of about 20 μg/L or about 250 mg/L;
linoleic fatty acid at a concentration of 10 μg/L-1 g/L, optionally at a concentration of about 100 μg/L or about 250 mg/L;
linolenic fatty acid at a concentration of 10 μg/L-1 g/L, optionally at a concentration of about 100 μg/L or about 250 mg/L;
myristic fatty acid at a concentration of 10 μg/L-1 g/L, optionally at a concentration of about 100 μg/L or about 250 mg/L;
oleic fatty acid at a concentration of 10 μg/L-1 g/L, optionally at a concentration of about 100 μg/L or about 250 mg/L;
palmitic fatty acid at a concentration of 10 μg/L-1 g/L, optionally at a concentration of about 100 μg/L or about 250 mg/L;
stearic fatty acid at a concentration of 10 μg/L-1 g/L, optionally at a concentration of about 100 μg/L or about 250 mg/L; and
cholesterol at a concentration of 1-5 g/L, optionally at a concentration of about 2.2 g/L;
wherein the g/L denote concentration when the medium is diluted in a solvent.

31. The medium of claim 30, comprising 2, 3, 4, 5, 6, 7, or all of:
arachidonic fatty acid at a concentration of 5 μg/L-1 g/L, optionally at a concentration of about 20 μg/L or about 250 mg/L;
linoleic fatty acid at a concentration of 10 μg/L-1 g/L, optionally at a concentration of about 100 μg/L or about 250 mg/L;
linolenic fatty acid at a concentration of 10 μg/L-1 g/L, optionally at a concentration of about 100 μg/L or about 250 mg/L;
myristic fatty acid at a concentration of 10 μg/L-1 g/L, optionally at a concentration of about 100 μg/L or about 250 mg/L;
oleic fatty acid at a concentration of 10 μg/L-1 g/L, optionally at a concentration of about 100 μg/L or about 250 mg/L;
palmitic fatty acid at a concentration of 10 μg/L-1 g/L, optionally at a concentration of about 100 μg/L or about 250 mg/L;
stearic fatty acid at a concentration of 10 μg/L-1 g/L, optionally at a concentration of about 100 μg/L or about 250 mg/L; and
cholesterol at a concentration of 1-5 g/L, optionally at a concentration of about 2.2 g/L;
wherein the g/L denote concentration when the medium is diluted in a solvent.

32. The medium of claim 1, comprising 2, 3, 4, 5, 6 or all of biotin, calcium pantothenate, nicotinic acid, inositol, thiamin HCl, pyridoxine HCl, and para-aminobenzoic acid.

33. (canceled)

34. The medium of claim 32, comprising 2, 3, 4, 5, 6 or all of:
biotin at a concentration of 0.01-1.0 mg/L, optionally at a concentration of about 0.5 mg/L;
calcium pantothenate at a concentration of 0.1-5 mg/L, optionally at a concentration of about 1.0 mg/L;
nicotinic acid at a concentration of 0.1-5 mg/L, optionally at a concentration of about 1.0 mg/L;
inositol at a concentration of 10-50 mg/L, optionally at a concentration of about 25 mg/L;
thiamin HCl at a concentration of 0.1-5 mg/L, optionally at a concentration of about 1.0 mg/L;
pyridoxine HCl at a concentration of 0.1-5 mg/L, optionally at a concentration of about 1.0 mg/L; and
para-aminobenzoic acid at a concentration of 0.05-0.5 mg/L, optionally about 0.2 mg;
wherein the g/L denote concentration when the medium is diluted in a solvent.

35. The medium of claim 1, wherein the KH₂PO₄ is present at a concentration of 10-15 g/L, optionally about 12 g/L, wherein the g/L denote concentration when the medium is diluted in a solvent.

36. The medium of claim 1, wherein the MgSO₄ is present at a concentration of 3-7 g/L MgSO₄.7H₂O, optionally about 4.7 g/L MgSO₄.7H₂O, wherein the g/L denote concentration when the medium is diluted in a solvent.

37. The medium of claim 1, wherein the CaCl₂ is present at a concentration of 0.3-0.4 g/L CaCl₂.2H₂O, optionally about 0.36 g/L CaCl₂.2H₂O, wherein the g/L denote concentration when the medium is diluted in a solvent.

38. The medium of claim 1, further comprising one or more of copper, iodine, manganese, molybdenum, boron, cobalt, zinc, iron, and vanadate.

39. (canceled)

40. The medium of claim 38, further comprising one or more of:
copper at a concentration of 5-10 mg/L, optionally at a concentration of about 6.64 mg/L;
iodine at a concentration of 0.05-1.0 mg/L, optionally at a concentration of about 0.29 mg/L;
manganese at a concentration of 2-8 mg/L, optionally at a concentration of about 4.24 mg/L;
molybdenum at a concentration of 0.05-1.0 mg/L, optionally at a concentration of about 0.35 mg/L;
boron at a concentration of 0.005-0.1 mg/L, optionally at a concentration of about 0.02 mg/L;
cobalt at a concentration of 0.1-5.0 mg/L, optionally at a concentration of about 0.99 mg/L;
zinc at a concentration of 5-100 mg/L, optionally at a concentration of about 41.47 mg/L; and
iron at a concentration of 5-100 mg/L, optionally at a concentration of about 56.80 mg/L,
wherein the g/L denote concentration when the medium is diluted in a solvent.

41. (canceled)

42. The medium of claim 1, wherein the medium is in a liquid form, optionally wherein the medium is supplied as a concentrated liquid for dilution in a liquid.

43. The medium of claim 1, wherein the medium is in a powder form for reconstitution in a liquid.

44-49. (canceled)

50. The medium of claim 1, wherein the pH of the medium is 3-8, optionally wherein the pH of the medium is 6.5.

51-52. (canceled)

53. A method for improving cell growth or maintenance in vitro comprising
culturing a first cell population under a first condition,
obtaining and analyzing expression products from an aliquot of the cultured cell population, identifying expression products having an altered expression level relative to a control, and modifying the first culture condition by addition and/or deletion of one or more culture components based on the identity of the expression products having an altered expression level.

54-55. (canceled)

56. The method of claim 53, wherein the expression products having an altered expression level are involved in one or more of cellular metabolism, fatty acid synthesis, vitamin synthesis, amino acid synthesis, nucleotide synthesis, protein glycosylation, redox biochemistry, and electron transport.

57-66. (canceled)

67. The method of claim 53, wherein the first cell population is a Pichia pastoris strain, optionally wherein the first and second cell populations are different Pichia pastoris strains.

68-69. (canceled)

70. The method of claim 53, wherein the first culture condition is modified by addition or deletion of one or more culture components based on the identity of the expression products having an altered expression level, optionally wherein the first culture condition is modified by increasing or decreasing the concentration of one or more culture components.

71-76. (canceled)

77. The method of claim 53, wherein expression products having an altered expression level are defined as expression products having a log 2 fold change that is greater than 2 with an adjusted p-value of less than 0.05.

78-86. (canceled)

87. A method of culturing cells comprising:
introducing a population of cells into the medium of claim 1; and
growing the population of cells.

88-89. (canceled)

90. The method of claim 87, wherein the medium is supplemented with a first carbon source, optionally wherein the first carbon source is selected from methanol, glycerol, sorbitol, glucose, galactose, raffinose, sucrose, trehalose, lactic acid, ethanol, oleic acid, xylose, xylitol, inulin, gluconate, and fructose.

91. (canceled)

92. The method of claim 87, further comprising providing medium with a second carbon source, optionally wherein the second carbon source is selected from methanol, glycerol, sorbitol, glucose, galactose, raffinose, sucrose, trehalose, lactic acid, ethanol, oleic acid, xylose, xylitol, inulin, gluconate, and fructose.

93. (canceled)

94. The method of claim 87, wherein the medium is supplemented with an additive for induction of protein expression.

95-105. (canceled)

106. The medium of claim 1, wherein the medium is suitable for supporting recombinant protein expression in a yeast, optionally P. pastoris.
